SEQUENCE LISTING (142 sequences)

SEQ ID NO: 1 - protein, 27 aa, Gallus gallus
TMGEIYAKDQDPDSIICTTIVNTSTLK

SEQ ID NO: 2 - protein, 29 aa, Homo sapiens
TMGEQNRSADGQHAGGLVCTPTIHTATVK

SEQ ID NO: 3 - protein, 29 aa, Cricetulus griseus
TMGLQNRSADGQHPGGLVCTPIVDTATVK

SEQ ID NO: 4 - protein, 29 aa, Cavia porcellus
TMGQQNRSADGQHPGGLVCTPTVDTATVK

SEQ ID NO: 5 - protein, 29 aa, Mus musculus
TMGLQNRSADGQHPGGLVCTPTVDTATVK

SEQ ID NO: 6 - protein, 29 aa, Rattus norvegicus
TMGLQNRSGDGTHPGGLVCTPIVDTATVK

SEQ ID NO: 7 - protein, 11 aa, Artificial Sequence
  Synthetic peptide
NRSADGTHPGG

SEQ ID NO: 8 - protein, 29 aa, Artificial Sequence
  Consensus sequence
TMGEQNRSADGQHPGGLVCTPTVDTATVK

SEQ ID NO: 9 - protein, 14 aa, Gallus gallus
SGGGSTDAGSYYYG

SEQ ID NO: 10 - protein, 7 aa, Gallus gallus
FNDKRPS

SEQ ID NO: 11 - protein, 9 aa, Gallus gallus
GSADSTGGI

SEQ ID NO: 12 - protein, 10 aa, Gallus gallus
GFTFSSFNMF

SEQ ID NO: 13 - protein, 18 aa, Gallus gallus
QIIDGAGSRTAYGAAVKG

SEQ ID NO: 14 - protein, 12 aa, Gallus gallus
GGHYWGGASIDA

SEQ ID NO: 15 - protein, 10 aa, Gallus gallus
GFTFSSFNRF

SEQ ID NO: 16 - protein, 10 aa, Gallus gallus
GFTVSSFNMF

SEQ ID NO: 17 - protein, 10 aa, Gallus gallus
GFTFSSFNMC

SEQ ID NO: 18 - protein, 10 aa, Gallus gallus
GFTFSSFNMV

SEQ ID NO: 19 - protein, 14 aa, Gallus gallus
SGGGSTDASSYYYG

SEQ ID NO: 20 - protein, 10 aa, Gallus gallus
GFTFSGFNMF

SEQ ID NO: 21 - protein, 10 aa, Gallus gallus
GFTFRSFNMF

SEQ ID NO: 22 - protein, 18 aa, Gallus gallus
QISDGAGSRTAYGAAVKG

SEQ ID NO: 23 - protein, 10 aa, Gallus gallus
GFTFSRFNMF

SEQ ID NO: 24 - protein, 10 aa, Gallus gallus
GVTFSSFNMF

SEQ ID NO: 25 - protein, 10 aa, Gallus gallus
GFTFSSVNMF

SEQ ID NO: 26 - protein, 10 aa, Gallus gallus
GFTFSSLNMF

SEQ ID NO: 27 - protein, 10 aa, Gallus gallus
GFTFGSFNMF

SEQ ID NO: 28 - protein, 10 aa, Gallus gallus
GFTFSSCNMF

SEQ ID NO: 29 - protein, 18 aa, Gallus gallus
QSIDGAGSRTAYGAAVKG

SEQ ID NO: 30 - protein, 18 aa, Gallus gallus
QMIDGAGSRTAYGAAVKG

SEQ ID NO: 31 - protein, 18 aa, Gallus gallus
QIIDGAGSRTADGAAVKG

SEQ ID NO: 32 - protein, 18 aa, Gallus gallus
QIIEGAGSRTAYGAAVKG

SEQ ID NO: 33 - protein, 108 aa, Gallus gallus
ALTQPSSVSANLGGTVKITCSGGGSTDAGSYYYGWYQQKAPGSAPVTVIYFNDKRPSGIPSRFSGSTSGSTGTLTITGVQADDEAVYFCGSADSTGGIFGAGTTLTVL

SEQ ID NO: 34 - protein, 108 aa, Gallus gallus
ALTQPSSVSANLGGTVKITCSGGGSTDAGSYYYGWYQQKSPGSAPVTVIYFNDKRPSDIPSRFSGSGSGSTGTLTITGVQAEDEAVYFCGSADSTGGIFGAGTTLTVL

SEQ ID NO: 35 - protein, 108 aa, Gallus gallus
ALTQPSSVSANLGGTVKITCSGGGSTDAGSYYYGWHQQKSPGSAPVTLIYFNDKRPSDIPSRFSGSKSGSTGTLTITGVRAEDEAVYYCGSADSTGGIFGAGTTLTVL

SEQ ID NO: 36 - protein, 108 aa, Gallus gallus
ALTQPSSVSANLGGTVKITCSGGGSTDAGSYYYGWYQQKSPGSAPVTLIYFNDKRPSDIPSRFSGSKSGSTATLTITGVQADDEAVYFCGSADSTGGIFGAGTTLTVL

SEQ ID NO: 37 - protein, 108 aa, Gallus gallus
ALTQPSSVSANLGGTVKITCSGGGSTDAGSYYYGWFQQKSPGSAPVTLIYFNDKRPSDIPSRFSGSASGSTATLTITGVQADDEAVYFCGSADSTGGIFGAGTTLTVL

SEQ ID NO: 38 - protein, 108 aa, Gallus gallus
ALTQPSSVSANLGGTVKITCSGGGSTDAGSYYYGWFQQKSPGSAPVTLIYFNDKRPSDIPSRFSGVLSGSTGTLTITGVRAEDEAVYYCGSADSTGGIFGAGTTLTVL

SEQ ID NO: 39 - protein, 108 aa, Gallus gallus
ALTQPSSVSANLGGTVKITCSGGGSTDAGSYYYGWYQQKSPGSAPVTVIYFNDKRPSGIPSRFSGSKSGSTGTLTITGVQAEDEAVYYCGSADSTGGIFGAGTTLTVL

SEQ ID NO: 40 - protein, 108 aa, Gallus gallus
ALTQPSSVSANLGGTVKITCSGGGSTDAGSYYYGWYQQKSPGSAPVTVIYFNDKRPSDIPSRFSGSTSGSTATLTITGVQAEDEAVYYCGSADSTGGIFGAGTTLTVL

SEQ ID NO: 41 - protein, 107 aa, Gallus gallus
ALTQPSSVSANLGGTVKITCSGGGSTDAGSYYYGWYQQKSPGSAPVTLIYFNDKRPSNIPSRFSGSASGSTATLTITGVQAEDEAYYCGSADSTGGIFGAGTTLTVL

SEQ ID NO: 42 - protein, 108 aa, Gallus gallus; identical to SEQ ID NO: 34

SEQ ID NO: 43 - protein, 108 aa, Gallus gallus; identical to SEQ ID NO: 35

SEQ ID NO: 44 - protein, 108 aa, Gallus gallus
ALTQPSSVSANLGGTVKITCSGGGSTDAGSYYYGWYQQKSPGSAPVTVIYFNDKRPSDIPSRFSGSTSGSTHTLTITGVQAEDEAVYFCGSADSTGGIFGAGTTLTVL

SEQ ID NO: 45 - protein, 108 aa, Gallus gallus
ALTQPSSVSANLGGTVKITCSGGGSTDAGSYYYGWYQQKSPGSAPVTLIYFNDKRPSDIPSRFSGSKSGSTGTLTITGVQAEDEAVYFCGSADSTGGIFGAGTTLTVL

SEQ ID NO: 46 - protein, 108 aa, Gallus gallus
ALTQPSSVSANLGRTVEITCSGGGSTDAGSYYYGWFQQKAPGSAPVTVIYFNDKRPSDIPSRFSGSASGSTGTLTITGVQAEDEAVYYCGSADSTGGIFGAGTTLTVL

SEQ ID NO: 47 - protein, 108 aa, Gallus gallus
ALTQPSSVSANLGGTVKITCSGGGSTDAGSYYYGWYQQKAPGSAPVTVIYFNDKRPSGIPSRFSGSKSGSTATLTITGVQAEDEAVYYCGSADSTGGIFGAGTTLTVL

SEQ ID NO: 48 - protein, 108 aa, Gallus gallus
ALTQPSSVSANLGGTVEITCSGGGSTDAGSYYYGWYQQKSPGSAPVTLIYFNDKRPSDIPSRFSGSTSGSTATLTITGVQADDEAVYYCGSADSTGGIFGAGTTLTVL

SEQ ID NO: 49 - protein, 108 aa, Gallus gallus
ALTQPSSVSANLGGTVKITCSGGGSTDAGSYYYGWYQQKSPGSAPVTVIYFNDKRPSNIPSRFSGSTSGSTATLTITGVQAEDEAVYFCGSADSTGGIFGAGTTLTVL

SEQ ID NO: 50 - protein, 108 aa, Gallus gallus
ALTQPSSVSANLGGTVKITCSGGGSTDAGSYYYGWFQQKSPGSALVTVIYFNDKRPSDIPSRFSGSTSGSTATLTITGVQADDEAVYYCGSADSTGGIFGAGTTLTVL

SEQ ID NO: 51 - protein, 108 aa, Gallus gallus
ALTQPSSVSANLGGTVKITCSGGGSTDAGSYYYGWYQQKSPGSAPVTVIYFNDKRPSDIPSRFSGSTSGSTATLTITGVQADDEAVYYCGSADSTGGIFGAGTTLTVL

SEQ ID NO: 52 - protein, 107 aa, Gallus gallus
ALTQPSSVSANLGGTVKITCSGGGSTDAGSYYYGWYQQKSPGSAPVTVIYFNDKRPSNIPSRFSGSASGSTATLTITGVQAEDEAYYCGSADSTGGIFGAGTTLTVL

SEQ ID NO: 53 - protein, 122 aa, Gallus gallus
AVTLDESGGGLQTPGRALSLVCKASGFTFSSFNMFWVRQAPGKGLEYVSQIIDGAGSRTAYGAAVKGRATISRDNGQSTVRLQLNNLRAEDTGTYYCAKGGHYWGGASIDAWGHGTEVIVSS

SEQ ID NO: 54 - protein, 122 aa, Gallus gallus
AVTLDESGGGLQTPGRALSLVCKASGFTFSSFNMFWVRQAPGKGLEYVAQIIDGAGSRTAYGAAVKGRATISRDNGQSTVRLQLNNLRAEDTGTYYCTKGGHYWGGASIDAWGHGTEVIVSS

SEQ ID NO: 55 - protein, 122 aa, Gallus gallus
AVTLDESGGGLQTPGRALSLVCKASGFTFSSFNMFWVRQAPGKGLEYVAQIIDGAGSRTAYGAAVKGRATISRDNGQSTVRLQLNNLRAEDTGIYFCAKGGHYWGGASIDAWGHGTEVIVSS

SEQ ID NO: 56 - protein, 122 aa, Gallus gallus
AVTLDESGGGLQTPGRALSLVCKASGFTFSSFNMFWVRQAPGKGLEFVAQIIDGAGSRTAYGAAVKGRATISRDNGQSTVKLQLNNLRAEDTGTYFCAKGGHYWGGASIDAWGHGTEVIVSS

SEQ ID NO: 57 - protein, 122 aa, Gallus gallus
AVTLDESGGGLQTPGRALSLVCKASGFTFSSFNMFWVRQAPGKGLEWVAQIIDGAGSRTAYGAAVKGRATISRDNGQSTVKLQLNNLRAEDTGTYYCAKGGHYWGGASIDAWGHGTEVIVSS

SEQ ID NO: 58 - protein, 122 aa, Gallus gallus
AVTLDESGGGLQTPGRALSLVCKASGFTFSSFNMFWVRQAPGKGLEWVAQIIDGAGSRTAYGAAVKGRATISRDNGQSTVKLQLNNLRAEDTATYYCARGGHYWGGASIDAWGHGTEVIVSS

SEQ ID NO: 59 - protein, 122 aa, Gallus gallus
AVTLDESGGGLQTPGRALSLVCKASGFTFSSFNMFWVRQAPGKGLEYVAQIIDGAGSRTAYGAAVKGRATISRDNGQSTVRLQLNNLRAEDTGTYFCAKGGHYWGGASIDAWGHGTEVIVSS

SEQ ID NO: 60 - protein, 122 aa, Gallus gallus
AVTLDESGGGLQTPGRALSLVCKASGFTFSSFNMFWVRQTPGKGLEYVAQIIDGAGSRTAYGAAVKGRATISKDNGQSTLRLQLNNLRAEDTATYYCAKGGHYWGGASIDAWGHGTEVIVSS

SEQ ID NO: 61 - protein, 122 aa, Gallus gallus
AVTLDESGGGLQTPGRALSLVCKASGFTFSSFNMFWVRQTPGKGLEYVAQIIDGAGSRTAYGAAVKGRATISRDNGQSTVRLQLNNLRAEDTGTYYCTRGGHYWGGASIDAWGHGTEVIVSS

SEQ ID NO: 62 - protein, 122 aa, Gallus gallus
AVTLDESGGGLQTPGRALSLVCKASGFTFSSFNMFWVRQTPGKGLEYVAQIIDGAGSRTAYGAAVKGRATISRDNGQSTVRLQLNNLRAEDTGTYYCTKGGHYWGGASIDAWGHGTEVIVSS

SEQ ID NO: 63 - protein, 122 aa, Gallus gallus
AVTLDESGGGLQTPGRALSLVCKASGFTFSSFNMFWVRQTPGKGLEYVAQIIDGAGSRTAYGAAVKGRATISRDNGQSTVRLQLNNLRAEDTGIYFCCAGGHYWGGASIDAWGHGTEVIVSS

SEQ ID NO: 64 - protein, 122 aa, Gallus gallus
AVTLDESGGGLQTPGRALSLVCKASGFTFSSFNMFWVRQTPGKGLEWVAQIIDGAGSRTAYGAAVKGRATISRDNGQSTVRLQLNNLRAEDTAIYYCAKGGHYWGGASIDAWGHGTEVIVSS

SEQ ID NO: 65 - protein, 122 aa, Gallus gallus; identical to SEQ ID NO: 62

SEQ ID NO: 66 - protein, 122 aa, Gallus gallus
AVTLDESGGGLQTPGRALSLVCKASGFTFSSFNMFWVRQTPGKGLDWVAQIIDGAGSRTAYGAAVKGRATISRDNGQSTVKLQLNNLRAEDTGTYYCARGGHYWGGASIDAWGHGTEVIVSS

SEQ ID NO: 67 - protein, 122 aa, Gallus gallus
AVTLDESGGGLQTPGRALSLVCKASGFTFSSFNMFWVRQTPGKGLEYVAQIIDGAGSRTAYGAAVKGRATISRDNGQSTVKLQLNNLRAEDTATYFCAKGGHYWGGASIDAWGHGTEVIVSS

SEQ ID NO: 68 - protein, 122 aa, Gallus gallus
AVTLDESGGGLQTPGRALSLVCKASGFTFSSFNMFWVRQTPGKGLEWVAQIIDGAGSRTAYGAAVKGRATISRDNGQSTVKLQLNNLRAEDTGIYYCTRGGHYWGGASIDAWGHGTEVIVSS

SEQ ID NO: 69 - protein, 122 aa, Gallus gallus
AVTLDESGGGLQTPGRALSLVCKASGFTFSSFNMFWVRQTPGKGLEWVAQIIDGAGSRTAYGAAVKGRATISRDNGQSTVRLQLNNLRAEDTGTYYCAKGGHYWGGASIDAWGHGTEVIVSS

SEQ ID NO: 70 - protein, 122 aa, Gallus gallus
AVTLDESGGGLQTPGRALSLVCKASGFTFSSFNMFWVRQTPGKGLEWVAQIIDGAGSRTAYGAAVKGRATISRDNGQSTVRLQLNNLRAEDTGTYYCARGGHYWGGASIDAWGHGTEVIVSS

SEQ ID NO: 71 - protein, 122 aa, Gallus gallus
AVTLDESGGGLQTPGRALSLVCKASGFTFSSFNMFWVRQAPGKGLEFVAQIIDGAGSRTAYGAAVKGRATISRDNGQSTVRLQLNNLRAEDTATYYCAKGGHYWGGASIDAWGHGTEVIVSS

SEQ ID NO: 72 - protein, 122 aa, Gallus gallus
AVTLDESGGGLQTPGRALSLVCKASGFTFSSFNMFWVRQTPGKGLEWLAQIIDGAGSRTAYGAAVKGRATISRDNGQSTVRLQLNNLRAEDTATYYCAKGGHYWGGASIDAWGHGTEVIVSS

SEQ ID NO: 73 - protein, 108 aa, Gallus gallus
ALTQPTSVSANLGGTVEITCSGGGSTDAGSYYYGWFQQKSPGSAPVTVIYFNDKRPSDIPSRFSGSTSGSTNTLTITGVQADDEAVYFCGSADSTGGIFGAGTTLTVL

SEQ ID NO: 74 - protein, 122 aa, Gallus gallus
AVTLDESGGGLQTPGGALSLVCKASGFTFSSFNMFWVRQAPGKGLEFVAQIIDGAGSRTAYGAAVKGRATISRDNGQSTVRLQLNNLRAEDTGTYYCARGGHYWGGASIDAWGHGTEVIVSS

SEQ ID NO: 75 - protein, 109 aa, Artificial Sequence
  Humanized antibody
SSELTQDPAVSVALGQTVRITCSGGGSTDAGSYYYGWYQQKPGQAPVTVIYFNDKRPSGIPDRFSGSSSGNTASLTITGAQAEDEADYYCGSADSTGGIFGGGTKLTVL

SEQ ID NO: 76 - protein, 122 aa, Artificial Sequence
  Humanized heavy chain
EVQLLESGGGLVQPGGSLRLSCAASGFTFSSFNMFWVRQAPGKGLEWVAQIIDGAGSRTAYGAAVKGRFTISRDNSKNTLYLQMNSLRAEDTAVYYCAKGGHYWGGASIDAWGQGTLVTVSS

SEQ ID NO: 77 - protein, 249 aa, Artificial Sequence
  Humanized scFV
SSELTQDPAVSVALGQTVRITCSGGGSTDAGSYYYGWYQQKPGQAPVTVIYFNDKRPSGIPDRFSGSSSGNTASLTITGAQAEDEADYYCGSADSTGGIFGGGTKLTVLGQSSRSSGGGGSSGGGGSEVQLLESGGGLVQPGGSLRLSCAASGFTFSSFNMFWVRQAPGKGLEWVAQIIDGAGSRTAYGAAVKGRFTISRDNSKNTLYLQMNSLRAEDTAVYYCAKGGHYWGGASIDAWGQGTLVTVSS

SEQ ID NO: 78 - protein, 238 aa, Artificial Sequence
  Humanized scFv
SSELTQDPAVSVALGQTVRITCSGGGSTDAGSYYYGWYQQKPGQAPVTVIYFNDKRPSGIPDRFSGSSSGNTASLTITGAQAEDEADYYCGSADSTGGIFGGGTKLTVLGQSSRSSEVQLLESGGGLVQPGGSLRLSCAASGFTFSSFNMFWVRQAPGKGLEWVAQIIDGAGSRTAYGAAVKGRFTISRDNSKNTLYLQMNSLRAEDTAVYYCAKGGHYWGGASIDAWGQGTLVTVSS

SEQ ID NO: 79 - protein, 233 aa, Homo sapiens
PEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 418 aa, Homo sapiens
MRLNSSAPGTPGTPAADPFQRAQAGLEEALLAPGFGNASGNASERVLAAPSSELDVNTDIYSKVLVTAVYLALFVVGTVGNTVTAFTLARKKSLQSLQSTVHYHLGSLALSDLLTLLLAMPVELYNFIWVHHPWAFGDAGCRGYYFLRDACTYATALNVASLSVERYLAICHPFKAKTLMSRSRTKKFISAIWLASALLAVPMLFTMGEQNRSADGQHAGGLVCTPTIHTATVKVVIQVNTFMSFIFPMVVISVLNTIIANKLTVMVRQAAEQGQVCTVGGEHSTFSMAIEPGRVQALRHGVRVLRAVVIAFVVCWLPYHVRRLMFCYISDEQWTPFLYDFYHYFYMVTNALFYVSSTINPILYNLVSANFRHIFLATLACLCPVWRRRRKRPAFSRKADSVSSNHTLSSNATRETLY

SEQ ID NO: 81 - protein, 424 aa, Rattus norvegicus
MHLNSSVPQGTPGEPDAQPFSGPQSEMEATFLALSLSNGSGNTSESDTAGPNSDLDVNTDIYSKVLVTAIYLALFVVGTVGNSVTAFTLARKKSLQSLQSTVHYHLGSLALSDLLILLLAMPVELYNFIWVHHPWAFGDAGCRGYYFLRDACTYATALNVASLSVERYLAICHPFKAKTLMSRSRTKKFISAIWLASALLAIPMLFTMGLQNRSGDGTHPGGLVCTPIVDTATVKVVIQVNTFMSFLFPMLVISILNTVIANKLTVMVHQAAEQGRVCTVGTHNGLEHSTFNMTIEPGRVQALRHGVLVLRAVVIAFVVCWLPYHVRRLMFCYISDEQWTTFLFDFYHYFYMLTNALFYVSSAINPILYNLVSANFRQVFLSTLACLCPGWRHRRKKRPTFSRKPNSMSSNHAFSTSATRETLY

SEQ ID NO: 82 - protein, 424 aa, Mus musculus
MHLNSSVQQGAPSEPGAQPFPHPQFGLETMLLALSLSNGSGNSSESILEPNSNLDVNTDIYSKVLVTAVYLALFVVGTVGNSVTAFTLARKKSLQSLQSTVHYHLGSLALSDLLILLLAMPVELYNFIWVHHPWAFGDAGCRGYYFLRDACTYATALNVASLSVERYLAICHPFKAKTLMSRSRTKKFISAIWLASALLAVPMLFTMGLQNRSADGQHPGGLVCTPTVDTATVKVVIQVNTFMSFLFPMLIISILNTVIANKLTVMVHQAAEQGRGVCTVGTHNSLEHSTFNMSIEPGRVQALRHGVLVLRAVVIAFVVCWLPYHVRRLMFCYISDEQWTTFLFDFYHYFYMLTNALFYVSSAINPILYNLVSANFRQVFLSTLACLCPGWRRRRKKRPTFSRKPNSMSSNHAFSTSATRETLY

SEQ ID NO: 83 - protein, 11 aa, Homo sapiens
NRSADGQHAGG

SEQ ID NO: 84 - protein, 11 aa, Homo sapiens
NRSGDGTHPGG

SEQ ID NO: 85 - protein, 11 aa, Mus musculus
NRSADGQHPGG

SEQ ID NO: 86 - protein, 14 aa, Gallus gallus
  VARIANT (9)..(9): Xaa is any amino acid, preferably gly or ser
SGGGSTDAXSYYYG

SEQ ID NO: 87 - protein, 10 aa, Gallus gallus
  VARIANT (2)..(2): Xaa is any amino acid
  VARIANT (4)..(4): Xaa is any amino acid
  VARIANT (5)..(5): Xaa is any amino acid
  VARIANT (6)..(6): Xaa is any amino acid
  VARIANT (7)..(7): Xaa is any amino acid
  VARIANT (9)..(9): Xaa is any amino acid
  VARIANT (10)..(10): Xaa is any amino acid
GXTXXXXNXX

SEQ ID NO: 88 - protein, 10 aa, Gallus gallus
  VARIANT (2)..(2): Xaa is any amino acid, preferably valine or phenylalanine
GXTFSSFNMF

SEQ ID NO: 89 - protein, 10 aa, Gallus gallus
  VARIANT (4)..(4): Xaa is any amino acid, preferably valine or phenylalanine
GFTXSSFNMF

SEQ ID NO: 90 - protein, 10 aa, Gallus gallus
  VARIANT (5)..(5): Xaa is any amino acid, preferably serine, arginine or glycine
GFTFXSFNMF

SEQ ID NO: 91 - protein, 10 aa, Gallus gallus
  VARIANT (6)..(6): Xaa is any amino acid, preferably serine, arginine or glycine
GFTFSXFNMF

SEQ ID NO: 92 - protein, 10 aa, Gallus gallus
  VARIANT (7)..(7): Xaa is any amino acid, preferably phenylalanine, valine, leucine or cysteine
GFTFSSXNMF

SEQ ID NO: 93 - protein, 10 aa, Gallus gallus
  VARIANT (9)..(9): Xaa is any amino acid, preferably methionine or arginine
GFTFSSFNXF

SEQ ID NO: 94 - protein, 10 aa, Gallus gallus
  VARIANT (10)..(10): Xaa is any amino acid, preferably phenylalanine, valine, or cysteine
GFTFSSFNMX

SEQ ID NO: 95 - protein, 18 aa, Gallus gallus
  VARIANT (2)..(2): Xaa is any amin acid
  VARIANT (3)..(3): Xaa is any amin acid
  VARIANT (4)..(4): Xaa is any amin acid
  VARIANT (12)..(12): Xaa is any amin acid
QXXXGAGSRTAXGAAVKG

SEQ ID NO: 96 - protein, 18 aa, Gallus gallus
  VARIANT (2)..(2): Xaa is any amino acid, preferably isoleucine, serine or methionine
QXIDGAGSRTAYGAAVKG

SEQ ID NO: 97 - protein, 18 aa, Gallus gallus
  VARIANT (3)..(3): Xaa is any amino acid, preferably isoleucine or serine
QIXDGAGSRTAYGAAVKG

SEQ ID NO: 98 - protein, 18 aa, Gallus gallus
  VARIANT (4)..(4): Xaa is any amino acid, preferably aspartic or glutamic acid
QIIXGAGSRTAYGAAVKG

SEQ ID NO: 99 - protein, 18 aa, Gallus gallus
  VARIANT (12)..(12): Xaa is any amino acid, preferably tyrosine or aspartic acid
QIIDGAGSRTAXGAAVKG

SEQ ID NO: 100 - protein, 20 aa, Gallus gallus
ALTQPTSVSANLGGTVEITC

SEQ ID NO: 101 - protein, 16 aa, Gallus gallus
WFQQKSPGSAPVTVIY

SEQ ID NO: 102 - protein, 32 aa, Gallus gallus
DIPSRFSGSTSGSTNTLTITGVQADDEAVYFC

SEQ ID NO: 103 - protein, 10 aa, Gallus gallus
FGAGTTLTVL

SEQ ID NO: 104 - protein, 25 aa, Gallus gallus
AVTLDESGGGLQTPGGALSLVCKAS

SEQ ID NO: 105 - protein, 13 aa, Gallus gallus
WVRQAPGKGLEFV

SEQ ID NO: 106 - protein, 32 aa, Gallus gallus
RATISRDNGQSTVRLQLNNLRAEDTGTYYCAR

SEQ ID NO: 107 - protein, 11 aa, Gallus gallus
WGHGTEVIVSS

SEQ ID NO: 108 - protein, 108 aa, Gallus gallus; identical to SEQ ID NO: 73

SEQ ID NO: 109 - protein, 122 aa, Gallus gallus; identical to SEQ ID NO: 74

SEQ ID NO: 110 - protein, 22 aa, Homo sapiens
SSELTQDPAVSVALGQTVRITC

SEQ ID NO: 111 - protein, 15 aa, Homo sapiens
WYQQKPGQAPVTVIY

SEQ ID NO: 112 - protein, 32 aa, Homo sapiens
GIPDRFSGSSSGNTASLTITGAQAEDEADYYC

SEQ ID NO: 113 - protein, 10 aa, Homo sapiens
FGGGTKLTVL

SEQ ID NO: 114 - protein, 25 aa, Homo sapiens
EVQLLESGGGLVQPGGSLRLSCAAS

SEQ ID NO: 115 - protein, 13 aa, Homo sapiens
WVRQAPGKGLEWV

SEQ ID NO: 116 - protein, 32 aa, Homo sapiens
RFTISRDNSKNTLYLQMNSLRAEDTAVYYCAK

SEQ ID NO: 117 - protein, 11 aa, Homo sapiens
WGQGTLVTVSS

SEQ ID NO: 118 - protein, 109 aa, Artificial Sequence; identical to SEQ ID NO: 75
  humanized antibody

SEQ ID NO: 119 - protein, 122 aa, Artificial Sequence; identical to SEQ ID NO: 76
  humanized antibody

SEQ ID NO: 120 - protein, 7 aa, Artificial Sequence
  Synthetic linker
GQSSRSS

SEQ ID NO: 121 - protein, 18 aa, Artificial Sequence
  GQSSRSSGGGGSSGGGGS
GQSSRSSGGGGSSGGGGS

SEQ ID NO: 122 - DNA, 30 bp, Artificial Sequence
  Synthetic oligonucleotide
tgcatctagagccaccatgtacccatacga

SEQ ID NO: 123 - DNA, 30 bp, Artificial Sequence
  Synthetic oligonucleotide
tcactgcagaattcctagtacagggtctcc

SEQ ID NO: 124 - protein, 248 aa, Artificial Sequence
  Chimeric antibody
ALTQPSSVSANLGGTVKITCSGSGSSWHGYGWYQQKAPGSAPVTVIYDNTNRPSNIPSRFSGSASGSTATLTITGVRAEDEAVYFCGGYDSSTYAGIFGAGTTLTVLGQSSRSSGGGGSSGGGGSAVTLDESGGGLQTPGRALSLVCKASGFTFSSFNMIWVRQTPGKGLEWVASICTGGSYTYYAPAVKGRATISRDNGQSTVRLQLNNLRAEDTATYFCAKSVDGVGWHAGQIDAWGHGTEVIVSS

SEQ ID NO: 125 - protein, 11 aa, Gallus gallus
SGSGSSWHGYG

SEQ ID NO: 126 - protein, 7 aa, Gallus gallus
DNTNRPS

SEQ ID NO: 127 - protein, 11 aa, Gallus gallus
GGYDSSTYAGI

SEQ ID NO: 128 - protein, 10 aa, Gallus gallus
GFTFSSFNMI

SEQ ID NO: 129 - protein, 18 aa, Gallus gallus
ASICTGGSYTYYAPAVKG

SEQ ID NO: 130 - protein, 14 aa, Gallus gallus
SVDGVGWHAGQIDA

SEQ ID NO: 131 - protein, 20 aa, Homo sapiens
ALTQPSSVSANLGGTVKITC

SEQ ID NO: 132 - protein, 16 aa, Homo sapiens
WYQQKAPGSAPVTVIY

SEQ ID NO: 133 - protein, 32 aa, Homo sapiens
NIPSRFSGSASGSTATLTITGVRAEDEAVYFC

SEQ ID NO: 134 - protein, 10 aa, Homo sapiens
FGAGTTLTVL

SEQ ID NO: 135 - protein, 25 aa, Homo sapiens
AVTLDESGGGLQTPGRALSLVCKAS

SEQ ID NO: 136 - protein, 13 aa, Homo sapiens
WVRQTPGKGLEWV

SEQ ID NO: 137 - protein, 32 aa, Homo sapiens
RATISRDNGQSTVRLQLNNLRAEDTATYFCAK

SEQ ID NO: 138 - protein, 10 aa, Homo sapiens
GHGTEVIVSS

SEQ ID NO: 139 - protein, 107 aa, Gallus gallus
ALTQPSSVSANLGGTVKITCSGSGSSWHGYGWYQQKAPGSAPVTVIYDNTNRPSNIPSRFSGSASGSTATLTITGVRAEDEAVYFCGGYDSSTYAGIFGAGTTLTVL

SEQ ID NO: 140 - protein, 123 aa, Gallus gallus
AVTLDESGGGLQTPGRALSLVCKASGFTFSSFNMIWVRQTPGKGLEWVASICTGGSYTYYAPAVKGRATISRDNGQSTVRLQLNNLRAEDTATYFCAKSVDGVGWHAGQIDAWGHGTEVIVSS

SEQ ID NO: 141 - protein, 108 aa, Artificial Sequence
  chimeric antibody
SSELTQPPAVSVALGQTVRITCSGSGSSWHGYGWYQQKPGQAPVTVIYDNTNRPSGIPDRFSGSSSGNTASLTITGAQAEDEADYYCGGYDSSTYAGIFGGGTKLTVL

SEQ ID NO: 142 - protein, 123 aa, Artificial Sequence
  chimeric antibody
EVQLLESGGGLVQPGGSLRLSCAASGFTFSSFNMIWVRQAPGKGLEWVASICTGGSYTYYAPAVKGRFTISRDNSKNTLYLQMNSLRAEDTAVYYCAKSVDGVGWHAGQIDAWGQGTLVTVSS

(19) United States
(12) Patent Application Publication
(10) Pub. No.: US 2021/0213118 A1
(43) Pub. Date: Jul. 15, 2021

(54) ANTIBODIES SPECIFIC FOR NTSR1 AND USES THEREOF

(71) Applicants: IMMUNE BIOSOLUTIONS INC, Sherbrooke (CA); SOCPRA SCIENCES SANTÉ ET HUMAINES S.E.C., Sherbrooke (CA)

(72) Inventors: Eric MARSAULT, Cherbrooke (CA); Pierre-Luc BOUDREAULT, Sherbrooke (CA); Hassan TRABOULSI, Al-Mubarraz (SA); Philippe SARRET, Sherbrooke (CA); Marie-Édith NEPVEU-TRAVERSY, Trois-Rivières (CA); Simon GAUDREAU, Sherbrooke (CA); Frédéric LEDUC, Sherbrooke (CA); Jeam-François LARRIVÉE, Sherbrooke (CA); Mathieu ARCAND, Sherbrooke (CA)

(21) Appl. No.: 17/250,734

(22) PCT Filed: Aug. 30, 2019
(86) PCT No.: PCT/CA2019/051216

§ 371 Date: Feb. 26, 2021

Related U.S. Application Data
(60) Provisional application No. 62/725,451, filed on Aug. 31, 2018.

(51) Int. Cl. A61K 39/00; A61P 35/00; C07K 16/28; A61K 47/64

(52) U.S. Cl. CPC A61K 39/001102; A61K 47/643; C07K 16/286; A61P 35/00

(57) ABSTRACT

Antibodies and antigen-binding fragments thereof that specifically recognize Neurotensin receptor type 1 (NTSR1) are described. These anti-NTSR1 antibodies and antigen-binding fragments thereof, such as single-chain Fv (scFv), are able to inhibit neurotensin-mediated activation of NTSR1 in normal and tumor cells. Methods and uses of antibodies and antigen-binding fragments thereof for treatment of diseases or conditions associated with NTSR1 activity, such as NTSR1-positive cancers or certain metabolic diseases, are also described. Cyclic peptides mimicking the conformation of the second extracellular loop of NTSR1 and capable of inducing the production of anti-NTSR1 antibodies in animals such as chickens are also described.

### CROSS REFERENCE TO RELATED APPLICATIONS

The present application claims the benefit of U.S. Provisional Patent Application Ser. No. 62/725,451 filed on Aug. 31, 2018, which is incorporated herein by reference in its entirety.

### TECHNICAL FIELD

The present invention generally relates to the field of Neurotensin receptor type 1 (NTSR1 or NTS1) modulation, for example using recombinant antibodies, for various applications, including the treatment of diseases or conditions associated with NTSR1 activity such as certain forms of cancers.

### BACKGROUND ART

NTSR1 belongs to the large superfamily of G-protein coupled receptors (GPCRs) and has been shown to mediate the multiple functions of neurotensin (NTS or NT), such as hypotension, hyperglycemia, hypothermia, antinociception, and regulation of intestinal motility and secretion. NTSR1 expression/overexpression has been shown to be associated with inflammatory bowel diseases (see, e.g., Gui et al., World J. Gastroenterol. 2013 Jul. 28; 19(28): 4504-4510. There is also evidence in the literature that NTSR1 activity is associated with neurological disorders, such as schizophrenia.

In addition to the physiological actions of NTSR1 engagement by NT in the central nervous system (CNS) and gastrointestinal tract, there is increasing evidence that NT-mediated NTSR1 stimulation plays an important role in carcinogenesis. NT oncogenic action has been described in numerous types of cancer cells and tumors with abnormal expression of NTSR1 (e.g., pancreatic, prostate, lung, breast, and colon cancer), with effects in each step of cancer progression from tumor growth, with proliferative and survival effects, to metastatic spread, with anchorage independent growth, and pro-migratory and pro-invasive effects (see, e.g., Wu et al., Front Endocrinol (Lausanne). 2012; 3: 184). All these effects are associated with the abnormal or dysregulated expression of NTSR1 during the early stages of cell transformation, and are the result from the activation of several kinases and effectors such as PKC, MAPK, FAK, RHO-GTPase, RAS and Scr (Younes et al., Oncotarget. 2014; 5(18): 8252-8269). NTSR1 expression has been shown to be an independent marker of poor prognosis in several cancers including breast, lung, and head and neck squamous cell carcinomas (Younes et al., 2014, supra; Ouyang et al., Mol Cancer. 2015; 14: 21; Alifano et al., Clin Cancer Res; 16(17); 4401-10).

Because of their important roles in intracellular signaling and their clinical relevance to a variety of diseases, including cancer, infection and inflammation, GPCRs such as NTSR1 are one of the most attractive therapeutic target classes. However, high conformational variability, the small exposed area of extracellular epitopes (which may not be accessible for antibody binding) as well as difficulties in the preparation of suitable GPCR antigens and efficient antibody screening tools have hampered the development of effective anti-GPCR antibodies, which remains a challenge. Indeed, no GPCR targeting antibody has been approved by the United States Food and Drug Administration and European Medicines Agency so far (Jo et al., Exp Mol Med. 2016 February; 48(2): e207).

Accordingly, there is a need for the development of agents that specifically target NTSR1, such as anti-NTRS1 antibodies, and for novel strategies for the treatment of diseases or conditions associated with NTSR1 activity, including certain form of cancers with poor prognosis.

The present description refers to a number of documents, the content of which is herein incorporated by reference in their entirety.

### SUMMARY OF THE INVENTION

The present disclosure provides the following items 1 to 91:
1. An antibody or an antigen binding fragment thereof that specifically binds to a conformational epitope located in an extracellular loop of Neurotensin receptor type 1 (NTSR1), wherein said extracellular loop corresponds to residues 206 to 234 of human NTSR1 (SEQ ID NO:80), and preferably to a conformational epitope located in the amino acid sequence NRSADGQHAGG from human Neurotensin receptor type 1 (NTSR1) and/or in the amino acid sequence NRSGDGTHPGG from rat NTSR1.
2. The antibody or an antigen binding fragment thereof according to item 1, which specifically binds to a conformational epitope located in the amino acid sequence NRSADGQHAGG from human NTSR1 and in the amino acid sequence NRSGDGTHPGG from rat NTSR1.
3. The antibody or an antigen binding fragment thereof according to item 1 or 2, which is a polyclonal antibody.
4. The antibody or an antigen binding fragment thereof according to item 1 or 2, which is a monoclonal antibody or an antigen binding fragment thereof.
5. The antibody or an antigen binding fragment thereof according to any one of items 1 to 4, which comprises the following combination of complementarity determining regions (CDRs): a light chain CDR1 (CDR-L1) comprising an amino acid sequence having at least 80% identity with the sequence SGGGSTDAGSYYYG (SEQ ID NO:9) or SGSGSSWHGYG (SEQ ID NO: 125); a CDR-L2 comprising an amino acid sequence having at least 80% identity with the sequence FNDKRPS (SEQ ID NO:10) or DNTNRPS (SEQ ID NO:126); a CDR-L3 comprising an amino acid sequence having at least 80% identity with the sequence GSADSTGGI (SEQ ID NO:11) or GGYDSSTYAGI (SEQ ID NO: 127); a heavy chain CDR1 (CDR-H1) comprising an amino acid sequence having at least 80% identity with the sequence GFTFSSFNMF (SEQ ID NO:12) or GFTFSSFNMI (SEQ ID NO: 128); a CDR-H2 comprising an amino acid sequence having at least 80% identity with the sequence AQIIDGAGSRTAYGAAVKG (SEQ ID NO:13) or ASICTGGSYTYYAPAVKG (SEQ ID NO:129); and a CDR-H3 comprising an amino acid sequence having at least 80% identity with the sequence GGHYWGGASIDA (SEQ ID NO:14) or SVDGVGWHAGQIDA (SEQ ID NO:130).
6. The antibody or an antigen binding fragment thereof according to item 5, wherein (i) the CDR-L1 comprises an amino acid sequence having at least 90% identity with the sequence SGGGSTDAGSYYYG (SEQ ID NO:9) or SGSGSSWHGYG (SEQ ID NO: 125); the CDR-L2 comprises an amino acid sequence having at least 80% identity with the sequence FNDKRPS (SEQ ID NO:10) or DNTNRPS (SEQ ID NO:126); the CDR-L3 comprises an amino acid sequence having at least 80% identity with the sequence GSADSTGGI (SEQ ID NO:11) or GGYDSSTYAGI (SEQ ID NO: 127); the CDR-H1 comprises an amino acid sequence having at least 80% identity with the sequence GFTFSSFNMF (SEQ ID NO:12) or GFTFSSFNMI (SEQ ID NO: 128); the CDR-H2 comprises an amino acid sequence having at least 80% identity with the sequence AQIIDGAGSRTAYGAAVKG (SEQ ID NO:13) or ASICTGGSYTYYAPAVKG (SEQ ID NO:129); and the CDR-H3 comprises an amino acid sequence having at least 80% identity with the sequence GGHYWGGASIDA (SEQ ID NO:14) or SVDGVGWHAGQIDA (SEQ ID NO:130), or (vii) any combination of (i) to (vi).
7. The antibody or an antigen binding fragment thereof according to item 6, wherein (i) the CDR-L1 comprises the sequence SGGGSTDAGSYYYG (SEQ ID NO:9), SGGGSTDASSYYYG (SEQ ID NO:19) or SGSGSSWHGYG (SEQ ID NO: 125), (ii) the CDR-L2 comprises the sequence FNDKRPS (SEQ ID NO:10) or DNTNRPS (SEQ ID NO:126), (iii) the CDR-L3 comprises the sequence GSADSTGGI (SEQ ID NO:11) or GGYDSSTYAGI (SEQ ID NO: 127), (iv) the CDR-H1 comprises the sequence GFTFSSFNMF (SEQ ID NO:12), GFTFSSFNRF (SEQ ID NO:15), GFTVSSFNMF (SEQ ID NO:16), GFTFSSFNMC (SEQ ID NO:17), GFTFSSFNMV (SEQ ID NO:18), GFTFSGFNMF (SEQ ID NO:20), GFTFRSFNMF (SEQ ID NO:21), GFTFSRFNMF (SEQ ID NO:23), GVTFSSFNMF (SEQ ID NO:24), GFTFSSVNMF (SEQ ID NO:25), GFTFSSLNMF (SEQ ID NO:26), GFTFGSFNMF (SEQ ID NO:27), GFTFSSCNMF (SEQ ID NO:28) or GFTFSSFNMI (SEQ ID NO: 128), (v) the CDR-H2 comprises the sequence QIIDGAGSRTAYGAAVKG (SEQ ID NO:13), QISDGAGSRTAYGAAVKG (SEQ ID NO:22), QSIDGAGSRTAYGAAVKG (SEQ ID NO:29), QMIDGAGSRTAYGAAVKG (SEQ ID NO:30), QIIDGAGSRTADGAAVKG (SEQ ID NO:31), QIIEGAGSRTAYGAAVKG (SEQ ID NO:32) or ASICTGGSYTYYAPAVKG (SEQ ID NO:129), (vi) the CDR-H3 comprises the sequence GGHYWGGASIDA (SEQ ID NO:14) or SVDGVGWHAGQIDA (SEQ ID NO:130), or (vii) any combination of (i) to (vi).
8. The antibody or an antigen binding fragment thereof according to item 7, wherein (i) the CDR-L1 comprises the sequence SGGGSTDAGSYYYG (SEQ ID NO:9), SGGGSTDASSYYYG (SEQ ID NO:19) or SGSGSSWHGYG (SEQ ID NO: 125), (ii) the CDR-L2 comprises the sequence FNDKRPS (SEQ ID NO:10) or DNTNRPS (SEQ ID NO:126), (iii) the CDR-L3 comprises the sequence GSADSTGGI (SEQ ID NO:11) or GGYDSSTYAGI (SEQ ID NO: 127), (iv) the CDR-H1 comprises the sequence GFTFSSFNMF (SEQ ID NO:12), GFTFSSFNRF (SEQ ID NO:15), GFTVSSFNMF (SEQ ID NO:16), GFTFSSFNMC (SEQ ID NO:17), GFTFSSFNMV (SEQ ID NO:18), GFTFSGFNMF (SEQ ID NO:20), GFTFRSFNMF (SEQ ID NO:21), GFTFSRFNMF (SEQ ID NO:23), GVTFSSFNMF (SEQ ID NO:24), GFTFSSVNMF (SEQ ID NO:25), GFTFSSLNMF (SEQ ID NO:26), GFTFGSFNMF (SEQ ID NO:27), GFTFSSCNMF (SEQ ID NO:28) or GFTFSSFNMI (SEQ ID NO: 128), (v) the CDR-H2 comprises the sequence QIIDGAGSRTAYGAAVKG (SEQ ID NO:13), QISDGAGSRTAYGAAVKG (SEQ ID NO:22), QSIDGAGSRTAYGAAVKG (SEQ ID NO:29), QMIDGAGSRTAYGAAVKG (SEQ ID NO:30), QIIDGAGSRTADGAAVKG (SEQ ID NO:31), QIIEGAGSRTAYGAAVKG (SEQ ID NO:32) or ASICTGGSYTYYAPAVKG (SEQ ID NO:129), and (vi) the CDR-H3 comprises the sequence GGHYWGGASIDA (SEQ ID NO:14) or SVDGVGWHAGQIDA (SEQ ID NO:130).
9. The antibody or an antigen binding fragment thereof according to any one of items 1 to 8, which is an IgY antibody or an antigen binding fragment thereof.
10. The antibody or an antigen binding fragment thereof according to item 9, wherein the antibody or antigen-binding fragment thereof comprises: (i) a light chain framework region (FR) 1 comprising an amino acid sequence having at least 60% identity with the sequence ALTQPTSVSANLGGTVEITC (SEQ ID NO:100) or ALTQPSSVSANLGGTVKITC (SEQ ID NO:131); (ii) a light chain FR2 comprising an amino acid sequence having at least 60% identity with the sequence WFQQKSPGSAPVTVIY (SEQ ID NO:101) or WYQQKAPGSAPVTVIY (SEQ ID NO:132); (iii) a light chain FR3 comprising an amino acid sequence having at least 60% identity with the sequence DIPSRFSGSTSGSTNTLTITGVQADDEAVYFC (SEQ ID NO:102) or NIPSRFSGSASGSTATLTITGVRAEDEAVYFC (SEQ ID NO:133); (iv) a light chain FR4 comprising an amino acid sequence having at least 60% identity with the sequence FGAGTTLTVL (SEQ ID NO:103) or FGAGTTLTVL (SEQ ID NO:134); or (v) any combination of (i) to (iv).
11. The antibody or an antigen binding fragment thereof according to item 10, wherein (i) the light chain FR1 comprises the sequence ALTQPTSVSANLGGTVEITC (SEQ ID NO:100) or ALTQPSSVSANLGGTVKITC (SEQ ID NO:131); (ii) the light chain FR2 comprises the sequence WFQQKSPGSAPVTVIY (SEQ ID NO:101) or WYQQKAPGSAPVTVIY (SEQ ID NO:132); (iii) the light chain FR3 comprises the sequence DIPSRFSGSTSGSTNTLTITGVQADDEAVYFC (SEQ ID NO:102) or NIPSRFSGSASGSTATLTITGVRAEDEAVYFC (SEQ ID NO:133); (iv) the light chain FR4 comprises the sequence FGAGTTLTVL (SEQ ID NO:103) or FGAGTTLTVL (SEQ ID NO:134); or (v) any combination of (i) to (iv).
12. The antibody or an antigen binding fragment thereof according to item 10, wherein (i) the light chain FR1 comprises the sequence ALTQPTSVSANLGGTVEITC (SEQ ID NO:100) or ALTQPSSVSANLGGTVKITC (SEQ ID NO:131); (ii) the light chain FR2 comprises the sequence WFQQKSPGSAPVTVIY (SEQ ID NO:101) or WYQQKAPGSAPVTVIY (SEQ ID NO:132); (iii) the light chain FR3 comprises the sequence DIPSRFSGSTSGSTNTLTITGVQADDEAVYFC (SEQ ID NO:102) or NIPSRFSGSASGSTATLTITGVRAEDEAVYFC (SEQ ID NO:133); and (iv) the light chain FR4 comprises the sequence FGAGTTLTVL (SEQ ID NO:103) or FGAGTTLTVL (SEQ ID NO:134).
13. The antibody or an antigen binding fragment thereof according to any one of items 9 to 12, wherein the antibody or antigen-binding fragment thereof comprises: (i) a heavy chain FR1 comprising an amino acid sequence having at least 60% identity with the sequence AVTLDESGGGLQTPGGALSLVCKAS (SEQ ID NO:104) or AVTLDESGGGLQTPGRALSLVCKAS (SEQ ID NO:135); (ii) a heavy chain FR2 comprising an amino acid sequence having at least 60% identity with the sequence WVRQAPGKGLEFV (SEQ ID NO:105) or WVRQTPGKGLEWV (SEQ ID NO:136); (iii) a heavy chain FR3 comprising an amino acid sequence having at least 60% identity with the sequence RATISRDNGQSTVRLQLNNLRAEDTGTYYCAR (SEQ ID NO:106) or RATISRDNGQSTVRLQLNNLRAEDTATYFCAK (SEQ ID NO:137); (iv) a heavy chain FR4 comprising an amino acid sequence having at least 60% identity with the sequence WGHGTEVIVSS (SEQ ID NO:107) or WGHGTEVIVSS (SEQ ID NO:138); or (v) any combination of (i) to (iv).
14. The antibody or an antigen binding fragment thereof according to item 13, wherein (i) the heavy chain FR1 comprises the sequence AVTLDESGGGLQTPGGALSLVCKAS (SEQ ID NO:104) or AVTLDESGGGLQTPGRALSLVCKAS (SEQ ID NO:135); (ii) the heavy chain FR2 comprises the sequence WVRQAPGKGLEFV (SEQ ID NO:105) or WVRQTPGKGLEWV (SEQ ID NO:136); (iii) the heavy chain FR3 comprises the sequence RATISRDNGQSTVRLQLNNLRAEDTGTYYCAR (SEQ ID NO:106); (iv) the heavy chain FR4 comprises the sequence WGHGTEVIVSS (SEQ ID NO:107); or (v) any combination of (i) to (iv).
15. The antibody or an antigen binding fragment thereof according to item 14, wherein (i) the heavy chain FR1 comprises the sequence AVTLDESGGGLQTPGGALSLVCKAS (SEQ ID NO:104); (ii) the heavy chain FR2 comprises the sequence WVRQAPGKGLEFV (SEQ ID NO:105); (iii) the heavy chain FR3 comprises the sequence RATISRDNGQSTVRLQLNNLRAEDTGTYYCAR (SEQ ID NO:106) or RATISRDNGQSTVRLQLNNLRAEDTATYFCAK (SEQ ID NO:137); and (iv) the heavy chain FR4 comprises the sequence WGHGTEVIVSS (SEQ ID NO:107) or WGHGTEVIVSS (SEQ ID NO:138).
16. The antibody or an antigen binding fragment thereof according to any one of items 9 to 15, wherein the antibody or antigen-binding fragment thereof comprises a variable light chain (V_{L}) comprising an amino acid sequence having at least 70% identity with the sequence

(SEQ ID NO: 108)
ALTQPTSVSANLGGTVEITCSGGGSTDAGSYYYGWFQQKSPGSAPVTVI
YFNDKRPSDIPSRFSGSTSGSTNTLTITGVQADDEAVYFCGSADSTGGI
FGAGTTLTVL
or
(SEQ ID NO: 139)
ALTQPSSVSANLGGTVKITCSGSGSSWHGYGWYQQKAPGSAPVTVIYDN
TNRPSNIPSRFSGSASGSTATLTITGVRAEDEAVYFCGGYDSSTYAGIF
GAGTTLTVL.

17. The antibody or an antigen binding fragment thereof according to item 16, wherein the V_{L} comprises the sequence of SEQ ID NO: 108 or 139.
18. The antibody or an antigen binding fragment thereof according to any one of items 9 to 17, wherein the antibody or antigen-binding fragment thereof comprises a variable heavy chain (V_{H}) comprising an amino acid sequence having at least 70% identity with the sequence

(SEQ ID NO: 109)
AVTLDESGGGLQTPGGALSLVCKASGFTFSSFNMFVVVRQAPGKGLEFV
AQIIDGAGSRTAYGAAVKGRATISRDNGQSTVRLQLNNLRAEDTGTYYC
ARGGHYWGGASIDAWGHGTEVIVSS
or
(SEQ ID NO: 140)
AVTLDESGGGLQTPGRALSLVCKASGFTFSSFNMIVVVRQTPGKGLEWV
ASICTGGSYTYYAPAVKGRATISRDNGQSTVRLQLNNLRAEDTATYFCA
KSVDGVGWHAGQIDAWGHGTEVIVSS.

19. The antibody or an antigen binding fragment thereof according to item 18, wherein the V_{H} comprises the sequence of SEQ ID NO:109 or 140.
20. The antibody or an antigen binding fragment thereof according to any one of items 1 to 8, which is a humanized form of an IgY antibody or an antigen binding fragment thereof.
21. The antibody or an antigen binding fragment thereof according to item 20, wherein the antibody or antigen-binding fragment thereof comprises: (i) a light chain framework region (FR) 1 comprising an amino acid sequence having at least 60% identity with the sequence SSELTQDPAVSVALGQTVRITC; (ii) a light chain FR2 comprising an amino acid sequence having at least 60% identity with the sequence WYQQKPGQAPVTVIY; (iii) a light chain FR3 comprising an amino acid sequence having at least 60% identity with the sequence GIPDRFSGSSSGNTASLTITGAQAEDEADYYC; (iv) a light chain FR4 comprising an amino acid sequence having at least 60% identity with the sequence FGGGTKLTVL; or (v) any combination of (i) to (iv).
22. The antibody or an antigen binding fragment thereof according to item 21, wherein (i) the light chain FR1 comprises the sequence SSELTQDPAVSVALGQTVRITC; (ii) the light chain FR2 comprises the sequence WYQQKPGQAPVTVIY; (iii) the light chain FR3 comprises the sequence GIPDRFSGSSSGNTASLTITGAQAEDEADYYC; (iv) the light chain FR4 comprises the sequence FGGGTKLTVL; or (v) any combination of (i) to (iv).
23. The antibody or an antigen binding fragment thereof according to item 22, wherein (i) the light chain FR1 comprises the sequence SSELTQDPAVSVALGQTVRITC; (ii) the light chain FR2 comprises the sequence WYQQKPGQAPVTVIY; (iii) the light chain FR3 comprises the sequence GIPDRFSGSSSGNTASLTITGAQAEDEADYYC; and (iv) the light chain FR4 comprises the sequence FGGGTKLTVL.
24. The antibody or an antigen binding fragment thereof according to any one of items 20 to 23, wherein the antibody or antigen-binding fragment thereof comprises: (i) a heavy chain FR1 comprising an amino acid sequence having at least 60% identity with the sequence EVQLLESGGGLVQPGGSLRLSCAAS; (ii) a heavy chain FR2 comprising an amino acid sequence having at least 60% identity with the sequence WVRQAPGKGLEWV; (iii) a heavy chain FR3 comprising an amino acid sequence having at least 60% identity with the sequence RFTISRDNSKNTLYLQMNSLRAEDTAVYYCAK; (iv) a heavy chain FR4 comprising an amino acid sequence having at least 60% identity with the sequence WGQGTLVTVSS; or (v) any combination of (i) to (iv).
25. The antibody or an antigen binding fragment thereof according to item 24, wherein (i) the heavy chain FR1 comprises the sequence EVQLLESGGGLVQPGGSLRLSCAAS; (ii) the heavy chain FR2 comprises the sequence WVRQAPGKGLEWV; (iii) the heavy chain FR3 comprises the sequence RFTISRDNSKNTLYLQMNSLRAEDTAVYYCAK; (iv) the heavy chain FR4 comprises the sequence WGQGTLVTVSS; or (v) any combination of (i) to (iv).
26. The antibody or an antigen binding fragment thereof according to item 25, wherein (i) the heavy chain FR1 comprises the sequence EVQLLESGGGLVQPGGSLRLSCAAS; (ii) the heavy chain FR2 comprises the sequence WVRQAPGKGLEWV; (iii) the heavy chain FR3 comprises the sequence RFTISRDNSKNTLYLQMNSLRAEDTAVYYCAK; and (iv) the heavy chain FR4 comprises the sequence WGQGTLVTVSS.
27. The antibody or an antigen binding fragment thereof according to any one of items 20 to 26, wherein the antibody or antigen-binding fragment thereof comprises a variable light chain (V_{L}) comprising an amino acid sequence having at least 70% identity with the sequence

(SEQ ID NO: 118)
SSELTQDPAVSVALGQTVRITCSGGGSTDAGSYYYGWYQQKPGQAPVTV
IYFNDKRPSGIPDRFSGSSSGNTASLTITGAQAEDEADYYCGSADSTGG
IFGGGTKLTVL
or
(SEQ ID NO: 141)
SSELTQPPAVSVALGQTVRITCSGSGSSWHGYGWYQQKPGQAPVTVIYD
NTNRPSGIPDRFSGSSSGNTASLTITGAQAEDEADYYCGGYDSSTYAGI
FGGGTKLTVL.

28. The antibody or an antigen binding fragment thereof according to item 27, wherein the V_{L} comprises the sequence of SEQ ID NO:118 or 141.
29. The antibody or an antigen binding fragment thereof according to any one of items 20 to 28, wherein the antibody or antigen-binding fragment thereof comprises a variable heavy chain (V_{H}) comprising an amino acid sequence having at least 70% identity with the sequence

(SEQ ID NO: 119)
EVQLLESGGGLVQPGGSLRLSCAASGFTFSSFNMFWVRQAPGKGLEWVA
QIIDGAGSRTAYGAAVKGRFTISRDNSKNTLYLQMNSLRAEDTAVYYCA
KGGHYWGGASIDAWGQGTLVTVSS
or
(SEQ ID NO: 142)
EVQLLESGGGLVQPGGSLRLSCAASGFTFSSFNMIWVRQAPGKGLEWVA
SICTGGSYTYYAPAVKGRFTISRDNSKNTLYLQMNSLRAEDTAVYYCAK
SVDGVGWHAGQIDAWGQGTLVTVSS.

30. The antibody or an antigen binding fragment thereof according to item 29, wherein the V_{H} comprises the sequence of SEQ ID NO:119 or 141.
31. The antibody or an antigen binding fragment thereof according to any one of items 1 to 30, which is an antigen binding fragment of an antibody.
32. The antibody or an antigen binding fragment thereof according to item 31, wherein said antigen binding fragment is a single-chain variable fragment (scFv) comprising a light chain variable region (V_{L}), a heavy chain variable region (V_{H}), and a linker connecting the V_{L} and the V_{H}.
33. The antibody or an antigen binding fragment thereof according to item 32, wherein said linker is a polypeptide linker comprising about 5 to about 50 amino acids.
34. The antibody or an antigen binding fragment thereof according to item 33, wherein said polypeptide linker comprises about 15 to about 20 amino acids.
35. The antibody or an antigen binding fragment thereof according to item 34, wherein said polypeptide linker comprises about 18 amino acids.
36. The antibody or an antigen binding fragment thereof according to any one of items 33 to 35, wherein at least 25% of the residues in the polypeptide linker are glycine residues.
37. The antibody or an antigen binding fragment thereof according to any one of items 33 to 36, wherein said linker comprises the pentapeptide sequence GGGGS.
38. The antibody or an antigen binding fragment thereof according to item 37, wherein the polypeptide linker comprises the sequence GQSSRSSGGGGSSGGGGS.
39. The antibody or an antigen binding fragment thereof according to any one of items 32 to 38, wherein said linker connects the N-terminal end of the V_{H} with the C-terminal end of the V_{L}.
40. The antibody or an antigen binding fragment thereof according to any one of items 1 to 39, wherein the antibody or antigen-binding fragment thereof comprises at least one constant domain or a fragment thereof.
41. The antibody or an antigen binding fragment thereof according to item 40, which comprises a Fragment crystallizable (Fc) fragment of a constant heavy chain of an antibody.
42. The antibody or an antigen binding fragment thereof according to item 41, wherein the Fc fragment comprises the sequence in bold in FIG. 8G.
43. The antibody or an antigen binding fragment thereof according to any one of items 1 to 42, which is conjugated to a detectable label or an affinity tag.
44. A nucleic acid or combination of nucleic acids encoding the antibody or an antigen binding fragment thereof according to any one of items 1 to 43.
45. A plasmid or vector comprising the nucleic acid or combination of nucleic acids according to item 44.
46. A host cell expressing the antibody or an antigen binding fragment thereof according to any one of items 1 to 43.
47. A composition comprising the antibody or an antigen-binding fragment thereof according to any one of items 1 to 43.
48. The composition according to item 47, further comprising a carrier or excipient, preferably a pharmaceutically acceptable carrier or excipient.
49. A method of detecting NTSR1 in a sample or on a cell comprising contacting the cell with the antibody or antigen-binding fragment thereof according to any one of items 1 to 43, or the composition according to item 47 or 48.
50. A method for inhibiting NTSR1 activity in a cell, the method comprising contacting the cell with an effective amount of the antibody or antigen-binding fragment thereof according to any one of items 1 to 43, or the composition according to item 47 or 48.
51. A method for inhibiting the activation of NTSR1 by neurotensin in a cell, the method comprising contacting the cell with an effective amount of the antibody or antigen-binding fragment thereof according to any one of items 1 to 43, or the composition according to item 47 or 48.
52. A method for treating a disease or condition associated with NTSR1 activity in a subject, the method comprising administering to the subject an effective amount of the anti-NTSR1 antibody or antigen-binding fragment thereof according to any one of items 1 to 43, or the composition according to item 47 or 48.
53. The method of item 52, wherein said disease or condition is an inflammatory bowel disease.
54. The method of item 52 or 53, wherein said treatment reduces the risk of developing colitis-associated neoplasia.
55. The method of item 52, wherein said disease or condition is a metabolic disease or condition.
56. The method of item 52, wherein said disease or condition is cancer, preferably pancreatic cancer, prostate cancer, lung cancer, breast cancer, thyroid cancer, liver cancer, brain cancer, uterine, skin cancer, gastric cancer or colorectal cancer.
57. The method of item 56, wherein said cancer is a primary cancer.
58. The method of item 56, wherein said cancer is a metastatic or secondary cancer.
59. The method of any one of items 56 to 58, wherein said cancer is a refractory cancer.
60. The method of any one of items 56 to 59, wherein said treatment is for reducing the cancer's aggressiveness and/or metastatic potential.
61. The method of any one of items 56 to 60, wherein said cancer expresses or overexpresses an epidermal growth factor receptor.
62. The method of item 61, wherein said epidermal growth factor receptor is a constitutively activated epidermal growth factor receptor.
63. The method of any one of items 56 to 62, wherein said antibody or antigen-binding fragment thereof is a scFv comprising a Fc fragment (scFV-Fc).
64. The method of any one of items 56 to 63, further comprising administering to the subject an effective amount of an additional chemotherapeutic agent.
65. The method of any one of items 52 to 64, wherein the subject is a human subject.
66. A cyclic peptide comprising a domain of the following sequence:

X¹—X²—X³—N—R—S-A-D-G¹-X⁴—H—X⁵-G²-G³
wherein
“—” are bonds, preferably peptide (amide) bonds;
X¹ is an amino acid or amino acid analog or is absent;
X² is an amino acid or amino acid analog or is absent;
X³ is an amino acid or amino acid analog or is absent;
X⁴ and X⁵ are any amino acid,
one of X³ or N is attached via a bond, preferably a peptide bond, to G³, thereby forming a cyclic peptide;
or a salt thereof.
67. The cyclic peptide of item 66, wherein X¹, if present, is cysteine.
68. The cyclic peptide of item 66 or 67, wherein X², if present, is a is a positively charged residue, preferably a lysine (K) residue.
69. The cyclic peptide of any one of items 66 to 68, wherein X³, if present, is a positively charged residue.
70. The cyclic peptide of item 69, wherein said positively charged residue is lysine (K) or ornithine.
71. The cyclic peptide of item 70, wherein the amine group of the lysine or ornithine forms a peptide bond with the carboxy-terminal end of G³.
72. The cyclic peptide of any one of items 66 to 71, wherein X⁵ is Q or T.
73. The cyclic peptide of item 72, wherein X⁵ is Q.
74. The cyclic peptide of item 72, wherein X⁵ is T.
75. The cyclic peptide of any one of items 66 to 74, wherein X⁶ is A or P.
76. The cyclic peptide of item 75, wherein X⁶ is A.
77. The cyclic peptide of item 75, wherein X⁶ is P.
78. The cyclic peptide of any one of items 66 to 77, which is conjugated to a carrier protein.
79. The cyclic peptide of item 78, wherein said carrier protein is keyhole limpet hemocyanin (KLH).
80. A composition comprising the cyclic peptide of any one of items 66 to 79.
81. The composition of item 80, further comprising a carrier or excipient, preferably a pharmaceutically acceptable carrier or excipient.
82. The composition of item 80 or 81, which is a vaccine or immunogenic composition.
83. The composition of item 82, wherein said vaccine composition comprises the cyclic peptide of item 78 or 79.
84. The composition of item 82 or 83, further comprising a vaccine adjuvant.
85. The composition of item 84, wherein the vaccine adjuvant is Freud's adjuvant.
86. A method for inducing the production of an antibody that specifically binds to NTSR1 in an animal, the method comprising administering to said animal an effective amount of the cyclic peptide according to any one of items 66 to 79, or the composition according to any one items 80 to 85, to said animal.
87. The method of item 86, further comprising collecting the antibody produced in the animal.
88. The method of item 87, further comprising purifying the antibody collected.
89. The method of item 88, wherein said purifying comprising submitting a sample comprising the antibody to an affinity purification step using the peptide administered to said animal.
90. The method of any one of items 86 to 89, wherein said animal is a chicken (hen).
91. The method of item 90, wherein said collecting of item 87 is from the eggs produced by said animal.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-D show the chemical structures of the four immunizing peptides (Lin-peptide, 3Dpeptide-1, 3Dpeptide-2 and 3Dpeptide-3, respectively) used in the studies described herein.

FIG. 2A is a graph showing the four inhibition curves of each IgY (-Lin, -3D-1, -3D-2 and -3D-3) binding to its corresponding bound-peptide by an increasing amount of the free-peptide using an ELISA-based procedure. The curves were evaluated by a nonlinear regression using 3 parameters. The curve fits yielded the IC₅₀ shown in the table under the graph.

FIG. 2B is a graph showing the inhibition of each IgY binding to its corresponding bound-peptide in a context of competition with the indicated free-peptide. The positive controls (black bars) correspond to a full detection of the specific bound-peptide by its corresponding IgY. All raw data measured at an absorbance of 450 nm were normalized on the control without peptide which corresponds to a detection of a 100%. Data represent mean±S.E.M. of at least three experimental replicates and are representative of at least two independent experiments.

FIG. 3A is a graph showing co-localization analysis (Fluoview FV10-ASW software) with the quantification of the Overlap coefficient of ten z-stack slices of five randomly chosen immunofluorescence microscopy fields considered as replicates. Immunofluorescence microscopy was performed on fixed HEK239A cells stably expressing the rat NTS1 receptor fused to hemagglutinin (HA-rNTSR1) with the indicated antibody and an anti-HA antibody to allow colocalization analysis (Fluoview FV10-ASW software). Pre-incubation of the antibodies with their corresponding peptides in a 1:10 ratio was used as a negative control.

FIG. 3B represents images of an immunohistochemistry assay performed on fixed rat brain slices with the indicated antibodies. IgY 3D-2 and IgY 3D-3 were also tested and resulted in a specific detection for the IgY-3D-3 only (not shown). Photos of the regions previously characterized for the expression of the rat NTS1 receptor (Boudin et al., The Journal of Comparative Neurology, 1996) are pictured and arrows are indicating the rNTS1 receptor signal. Experiments are representative of at least two independent experiments. The negative control corresponds to incubation with the secondary antibody only.

FIG. 4A shows an immunoblot (anti-HA HRP) of the immunoprecipitations used to isolate the rat NTS1 receptor, with the indicated chicken antibody conjugated to magnetic beads, for subsequent mass spectrometry identification. Negative controls consist in a pre-incubation of the IgY with its corresponding peptide in a 1:10 ratio.

FIG. 4B depicts the ion data of each amino acid of the identified Rattus norvegicus peptide sequence by mass spectrometry using the IgY-3D-1 and the IgY-3D-3.

FIG. 4C is a graph presenting the relative signal intensity of the identified rat peptide, calculated from the area under the curve of the spectrum obtained for each indicated condition (Peakview 2.2 software). All experiments are representative of at least two independent experiments. Mass spectrometry was performed and analysed by two different facilities.

FIG. 5A shows an alignment of the amino acid sequences of the second extracellular loop of NTSR1 from various species as well as the sequence of the peptide (epitope) used to immunize chickens in the studies described herein. The bars under the aligned sequences show the conservation percentage (CLC sequence bio V5).

FIGS. 5B and 5C are graphs showing the binding of the polyclonal IgY-3D-3 antibody to NTS1 protein mutants. Five NTS1 protein mutants were generated by substituting each of the five amino acids of the designed epitope (D-G-T-H—P) predicted to be the most exposed, by an alanine (A). All mutants were stably expressed in HEK cells. FIG. 5B: flow cytometry was performed on all live cells expressing either the WT NTS1 or one of the five mutants with the IgY-3D-3 antibody. Fluorescence (FITC) was evaluated for at least 25,000 cells per condition. The control (CTL) correspond to the WT cells incubated with the secondary antibody only. FIG. 5C: Binding of the IgY-3D-3 antibody was measured by the displacement of radiolabeled neurotensin (NT) on the six different membrane preparations from cells expressing the WT or the mutant form of NTS1. The control corresponds to the total binding with buffer only, without antibody.

FIGS. 5D and 5E are immunoblots showing the selectivity of the IgY-3D-1 and IgY-3D-3 chicken antibodies for rat NTSR1 vs. rat NTSR2. HEK293A cells transiently expressing HA-tagged rat NTS1, rat NTS2 or rat Apelin (APJ) receptor were processed for Western blots (FIG. 5D) and immunoprecipitations in native conditions (FIG. 5E) using either the IgY-3D-1 or the IgY-3D-3.

FIG. 6A is a graph showing displacement curves of radiolabeled-neurotensin by the chicken antibodies. The curves were evaluated by a nonlinear regression using three parameters. The curve fits yielded the IC₅₀ indicated in the legend. Data represent mean±S.E.M. of at least three experimental replicates and are representative of at least two independent experiments.

FIG. 6B is a graph showing the neurotensin-(NT) dependent cyclic AMP production blockade by the antibodies. The negative control corresponds to the non-stimulated cells and all antibodies were tested in presence of NT (EC₅₀). One-way ANOVA followed by a Turkey's multiple comparison tests was performed (Graphpad), **** corresponds to a P value <0.0001.

FIG. 6C is a graph showing the tested signaling pathways of the rat NTS1 receptor for their activity in response to the binding of the natural ligand, NT (at a dose corresponding to EC₅₀).

FIGS. 6D and 6E are graphs showing the capacity of the indicated antibodies to activate the G_{αq} (FIG. 6D) and β-arrestin-2 (FIG. 6E) signaling pathways dependent of the rat NTS1 receptor (agonist mode), as assessed using a BRET-based assay.

FIGS. 6F and 6G are graphs showing the capacity of the indicated antibodies to block the NT-mediated G_{αq} (FIG. 6F) and β-arrestin-2 (FIG. 6G) signaling pathways (antagonist mode, NT dose corresponding to EC₅₀), as assessed using a BRET-based assay. SR48692 (Meclinertant) is a well-documented antagonist of the NTS1 receptor and was used as a positive control. The curves were evaluated by a nonlinear regression using three parameters. Data are representative of at least two independent experiments.

FIG. 7A depicts a Western blot of the scFv (His-tagged) using an anti-scFv or an anti-His antibody coupled to HRP.

FIG. 7B shows an immunofluorescence microscopy image on fixed HEK cells stably expressing HA-human NTS1. NTS1 was detected using anti-HA (left panel) and scFv 238 at two different concentrations (1:200 and 1:50, middle panels). The right panel corresponds to the control with the secondary antibodies only (2^{nd} CTL).

FIGS. 7C and 7D show displacement curves of radiolabeled-neurotensin (¹²⁵I-└Tyr³┘NT) by an increasing amount of scFv 238 on HCT116 (FIG. 7C) and PANC-1 (FIG. 7D) membranes preparations. The curves were evaluated by a nonlinear regression using three parameters. The curve fits yielded the IC₅₀ indicated in the legend.

FIGS. 7E and 7F are graphs showing the blockade of IP-One production (%) (a measure of Gαq-mediated inositol monophosphate (IP1) production) following G_{αq} activation by NT 1-13 with an increasing amount of scFv 238 as measured by Homogeneous Time Resolved Fluorescence (HTRF) in HCT116 (FIG. 7E) and PANC-1 (FIG. 7F) live cells. SR48692 (10 μM) was used as an antagonist control. The curves were evaluated by a nonlinear regression using four parameters. The curve fits yielded the IC₅₀ indicated in the legend.

FIG. 7G is a graph depicting the selective NTS1 receptor blockade in HCT116 live cells by scFv 238. The G_{αq} pathway was activated with NT 1-13 (10 nM) and UDP (400 μM) and IP-one production was blocked by adding scFv 238 (2.8 μM). One-way ANOVA followed by a Tukey's multiple comparisons test was performed. Data represent mean±S.E.M. of at least three experimental replicates and are representative of at least two independent experiments.

FIG. 8A depicts an alignment of the amino acid sequences of the CDRs from 19 IgY antibodies obtained following immunization of chickens with the 3Dpeptide-3. The column entitled “#” corresponds the number of antibodies having the indicated CDR sequences that were sequenced.

FIG. 8B shows an alignment of the light chain variable region of 20 IgY antibodies obtained following immunization of chickens with the 3Dpeptide-3, with the CDR regions in bold and the amino acid variations in the framework regions underlined.

FIG. 8C shows an alignment of the heavy chain variable region of 20 IgY antibodies obtained following immunization of chickens with the 3Dpeptide-3, with the CDR regions in bold and the amino acid variations in the framework regions underlined.

FIG. 8D shows the amino acid sequences of the light and heavy chains of the most abundant IgY antibody produced in the studies described herein, with the different regions identified.

FIG. 8E shows the amino acid sequences of the light and heavy chains of a humanized form of the chicken antibody of FIG. 8D, with the different regions identified.

FIGS. 8F and 8G show the amino acid sequences of scFv fragments comprising the light and heavy chains of the humanized antibody of FIG. 8E connected by a long 18-amino acid linker (FIG. 8F, hereinafter referred to as scFv 238)) or a short 7-amino acid linker (FIG. 8G), with the different regions identified.

FIG. 8H shows the amino acid sequences of another scFv fragment (chicken, hereinafter referred to as scFv 009) prepared and used in studies described herein.

FIG. 8I shows the amino acid sequences of the hinge region (underlined) and Fc domain (bold) of the scFv-Fc antibody fragment described herein.

FIGS. 9A to 9C show the amino acid sequences of human, rat and mouse NTSR1, respectively, with the extracellular loop targeted by the antibodies described herein highlighted in grey and the region corresponding to the peptide used for producing the antibodies described herein in bold and underlined.

FIG. 10 shows the characteristics of the four immunizing peptides (lin, 3Dpeptide-1, 3Dpeptide-2 and 3Dpeptide-3) used in the studies described herein. The primary sequence of each peptide, the type of linker and the chain used to produce the macrocycle are indicated. Cross-reactivity with human NTSR1 and the functional properties of the four IgY antibodies, produced with the corresponding indicated peptides, are shown in the last two columns of the table (+ and +++ correspond to a low and high activity, respectively).

FIGS. 11A and 11B show displacement curves of radiolabeled-neurotensin (¹²⁵I-└Tyr³┘NT) by an increasing amount of scFv-Fc 238 and scFv-Fc 009 on HCT116 (FIG. 11A) and PANC-1 (FIG. 11B) membranes preparations. The curves were evaluated by a nonlinear regression using three parameters. The curve fits yielded the IC₅₀ indicated in the legend.

FIGS. 11C and 11D are graphs showing the blockade of IP-One production (%) following G_{αq} activation by NT 1-13 with an increasing amount of scFv-Fc 238 and scFv-Fc 009 as measured by HTRF in HCT116 (FIG. 11C) and PANC-1 (FIG. 11D) live cells. SR48692 (10 μM) was used as an antagonist control. The curves were evaluated by a nonlinear regression using four parameters. The curve fits yielded the IC₅₀ indicated in the legend. Data represent mean±S.E.M. of at least three experimental replicates and are representative of at least two independent experiments.

FIGS. 11E and 11F are graphs showing the antibody-dependent cellular cytotoxicity (ADCC) response of scFv-Fc 238 on live target HCT116 (FIG. 11E) and PANC-1 (FIG. 11F) cells. The ADCC response was measured with a reporter bioassay (NFAT gene activation in Jurkat effector cells) using increasing amounts of scFv-Fc 238 on live target HCT116 and PANC-1 cells. Total luminescence was normalized on the negative control (sample without antibody) and cetuximab (anti-EGFR chimeric monoclonal antibody) was used as a positive control for ADCC. The curves were evaluated by a non linear regression using three parameters. Resulting EC₅₀ are indicated in the legend. Data are representative of at least two independent experiments.

FIG. 12A depicts a Western blot of immunoprecipitation experiments with protein A/G agarose beads. Whole cell lysates from HEK stably expressing HA-human NTS1 or HEK CTL (not expressing NTS1, right panel) were incubated overnight with the beads and the tested antibodies (scFv-Fc 238 and 009 and a mouse anti-HA IgG). Western blot was revealed with an anti-HA HRP antibody.

FIG. 12B is a table showing the different patient-derived xenografts (PDX) tested for immunohistochemistry (IHC) using scFv-Fc 238 and 009. Related type of cancer is indicated. Positive staining was evaluated for all slides ranging from no staining (−) to strong staining (+++). (+/−) represents a staining slightly more intense than the IHC obtained with the isotypic antibody (human IgG).

FIG. 12C depicts images of the IHC obtained with the indicated antibodies (Isotypic, scFv-Fc 238 and 009) on the eight PDX. A goat anti-human IgG conjugated to HRP was used for staining and pictures were scanned at a 20× magnifying lense (Zeiss Axiolmager M2).

### DISCLOSURE OF INVENTION

In the studies described herein, the present inventors have engineered a cyclic peptide mimicking the conformation of the second extracellular loop of the NTS1 receptor from various species (including humans), which was used to immunize chicken and raise IgY antibodies specifically recognizing the NTS1 receptor. These antibodies were shown to compete with NT for binding to the NTS1 receptor, to inhibit NT-dependent cyclic AMP production as well as NT-mediated Gαq and β-arrestin-2 signaling pathway activation. Recombinant forms (scFv and scFv-Fc) of these antibodies were also produced, and were shown to inhibit neurotensin-mediated G_{αq} activation and trigger ADCC in cancer cell lines endogenously expressing human NTS1 receptor, and to bind to cancer patient-derived xenografts from different origins.

### Anti-NTSR1 Antibodies and Antigen-Binding Fragments Thereof

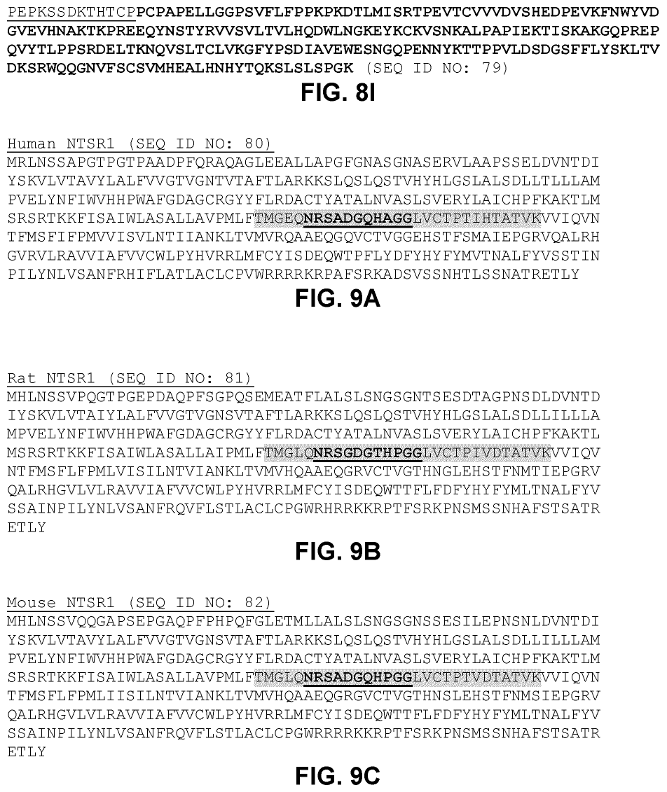

Accordingly, in a first aspect, the present disclosure provides an antibody or an antigen binding fragment thereof that specifically binds to an epitope (e.g., conformational epitope) located in an extracellular loop of the NTS1 receptor (NTSR1), preferably in an extracellular loop of NTSR1 corresponding to residues 206-234 of human NTSR1 (FIG. 9A, SEQ ID NO:80). This extracellular loop is defined by residues 207-235 in rat NTSR1 (FIG. 9B, SEQ ID NO:81) and residues 206-234 of mouse NTSR1 (FIG. 9C, SEQ ID NO:82). In another aspect, the present disclosure provides an antibody or an antigen binding fragment thereof that blocks the interaction between neurotensin and NTSR1. An embodiment, the antibody or antigen binding fragment thereof does not bind to NTS2.

In an embodiment, the antibody or an antigen binding fragment thereof specifically binds to a conformational epitope located in the amino acid sequence NRSADGQHAGG (SEQ ID NO:83) from human NTS1 receptor (NTSR1) and/or in the amino acid sequence NRSGDGTHPGG (SEQ ID NO:84) from rat NTSR1. In an embodiment, the antibody or antigen binding fragment thereof specifically binds to a conformational epitope located in the amino acid sequence NRSADGQHAGG (SEQ ID NO:83) from human NTSR1 and in the amino acid sequence NRSGDGTHPGG (SEQ ID NO:84) from rat NTSR1. In an embodiment, the antibody or antigen binding fragment thereof specifically binds to a conformational epitope located in the amino acid sequence NRSADGQHPGG (SEQ ID NO:85) from mouse NTSR1. In an embodiment, the antibody or antigen binding fragment thereof specifically binds to a conformational epitope located in the amino acid sequence NRSADGTHPGG (SEQ ID NO:86).

The term “conformational epitope” as used herein refers to an antigenic determinant found in the native, three-dimensional structure of a protein and capable of specific binding to an antibody or an antigen-binding fragment thereof.

The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)₂, and Fv fragments, diabodies, linear antibodies, single-chain antibody molecules (e.g., single-chain FV, scFV), single domain antibodies (e.g., from camelids), shark NAR single domain antibodies, and multispecific antibodies formed from antibody fragments. Antibody fragments can also refer to binding moieties comprising CDRs or antigen binding domains including, but not limited to, V_{H} regions (V_{H}, V_{H}—V_{H}), anticalins, PepBodies, antibody-T-cell epitope fusions (Troybodies) or Peptibodies.

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this invention. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N. Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5): 1073-1093 (2004); Fellouse, Proc. Nat. Acad. Sci. USA 101(34): 12467-12472 (2004); and Lee et al. J. Immunol. Methods 284(1-2):119-132 (2004) and technologies for producing human or human-like antibodies from animals that have parts or all of the human immunoglobulin loci or genes encoding human immunoglobulin sequences (see, e.g., WO98/24893, WO96/34096, WO96/33735, and WO91/10741, Jakobovits et al., Proc. Natl. Acad. Sci. USA, 90:2551 (1993); Jakobovits et al., Nature, 362:255-258 (1993); Bruggemann et al., Year in Immune, 7:33 (1993); U.S. Pat. Nos. 5,545,806, 5,569,825, 5,591,669 (all of GenPharm); U.S. Pat. No. 5,545,807; WO 97/17852, U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995). Antibodies capable of specifically binding to the conformational epitope from NTSR1 defined herein can also be produced using phage display technology. Antibody fragments that selectively bind to the conformational epitope from NTSR1 defined herein can then be isolated. Exemplary methods for producing such antibodies via phage display are disclosed, for example, in U.S. Pat. No. 6,225,447.

The monoclonal antibodies herein specifically include “chimeric” or “recombinant” antibodies in which a portion of the light and/or heavy chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “humanized” antibodies.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions (HVRs) both in the light-chain and heavy-chain variable domains. The more highly conserved portions of variable domains are called the framework region (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular cytotoxicity (ADCC). From N-terminal to C-terminal, both light and heavy chain variable regions comprise alternating FRs and CDRs: FR1, CDR1, FR2, CDR2, FR3, CDR3 and FR4. The assignment of amino acids to each region may be made in accordance with the definitions of Kabat, Chothia (Al-Lazikani et al., J Mol Biol. 1997; 273(4):927-48), or IMGT (Lefranc, M.-P., Immunology Today, 18, 509 (1997)), for example.

“Fv” is the minimum antibody fragment which contains a complete antigen-recognition and binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-V_{L} dimer. Collectively, the six CDRs are involved in conferring the antigen-binding specificity to the antibody. However, even a single variable domain (or half of an Fv comprising only three CDRs specific for an antigen) has the ability to recognize and bind antigen, although at a lower affinity than the entire binding site.

“Hypervariable region” or “HVR” refers to the amino acid residues of an antibody that are responsible for antigen-binding. The hypervariable region generally comprises amino acid residues from a “complementarity determining region” or “CDR” (Kabat et al., Sequences of Proteins of Immunological Interest, 5th Ed. Public Health Service, National Institutes of Health, Bethesda, Md. (1991)) and/or those residues from a “hypervariable loop” (Al-Lazikani et al., supra).

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptors variable domains, three loops coming from each of the two variable domains of the receptor.

As used herein, the term “framework region” refers to those portions of immunoglobulin light and heavy chain variable regions that are relatively conserved (i.e., other than the CDRs) among different immunoglobulins in a single species, as defined by Kabat et al. (supra) or Chothia (Al-Lazikani et al., supra). As used herein, a “human framework region” is a framework region that is substantially identical to the framework region of a naturally occurring human antibody.

In an embodiment, the anti-NTSR1 antibody or antigen-binding fragment thereof is a polyclonal antibody.

In another embodiment, the anti-NTSR1 antibody or antigen-binding fragment thereof is a monoclonal antibody.

In an embodiment, the antibody or antigen-binding fragment thereof comprises the following combination of complementarity determining regions (CDRs): a light chain CDR1 (CDR-L1) comprising or consisting of an amino acid sequence having at least 70%, 75%, 80%, 85% or 90% identity with the sequence SGGGSTDAGSYYYG (SEQ ID NO:9) or SGSGSSWHGYG (SEQ ID NO: 125); a CDR-L2 comprising or consisting of an amino acid sequence having at least 70%, 75%, 80%, 85% or 90% identity with the sequence FNDKRPS (SEQ ID NO:10) or DNTNRPS (SEQ ID NO:126); a CDR-L3 comprising or consisting of an amino acid sequence having at least 70%, 75%, 80%, 85% or 90% identity with the sequence GSADSTGGI (SEQ ID NO:11) or GGYDSSTYAGI (SEQ ID NO: 127); a heavy chain CDR1 (CDR-H1) comprising or consisting of an amino acid sequence having at least 70%, 75%, 80%, 85% or 90% identity with the sequence GFTFSSFNMF (SEQ ID NO:12) or GFTFSSFNMI (SEQ ID NO: 128); a CDR-H2 comprising or consisting of an amino acid sequence having at least 70%, 75%, 80%, 85% or 90% identity with the sequence QIIDGAGSRTAYGAAVKG (SEQ ID NO:13) or ASICTGGSYTYYAPAVKG (SEQ ID NO:129); and a CDR-H3 comprising or consisting of an amino acid sequence having at least 70%, 75%, 80%, 85% or 90% identity with the sequence GGHYWGGASIDA (SEQ ID NO:14) or SVDGVGWHAGQIDA (SEQ ID NO:130).

In an embodiment, one or two residues in the above-noted CDRs sequences are substituted. In a further embodiment, one residue in the above-noted CDRs sequences are substituted.

In an embodiment, the CDR-L1 comprises the following sequence: SGGGSTDAXSYYYG (SEQ ID NO:86), wherein X is any amino acid, preferably glycine or serine.

In an embodiment, the CDR-H1 comprises the following sequence: GX¹TX²X³X⁴X⁵NX⁶X⁷ (SEQ ID NO:87), wherein X¹ to X⁷ are independently any amino acids. In an embodiment, the CDR-H1 comprises the following sequence: GX¹TFSSFNMF (SEQ ID NO:88), wherein X¹ is any amino acid, preferably valine or phenylalanine. In an embodiment, the CDR-H1 comprises the following sequence: GFTX²SSFNMF (SEQ ID NO:89), wherein X² is any amino acid, preferably valine or phenylalanine. In an embodiment, the CDR-H1 comprises the following sequence: GFTFX³SFNMF (SEQ ID NO:90), wherein X³ is any amino acid, preferably serine, arginine or glycine. In an embodiment, the CDR-H1 comprises the following sequence: GFTFSX⁴FNMF (SEQ ID NO:91), wherein X⁴ is any amino acid, preferably serine, arginine or glycine. In an embodiment, the CDR-H1 comprises the following sequence: GFTFSSX⁵NMF (SEQ ID NO:92), wherein X⁵ is any amino acid, preferably phenylalanine, valine, leucine or cysteine. In an embodiment, the CDR-H1 comprises the following sequence: GFTFSSFNX⁶F (SEQ ID NO:93), wherein X⁶ is any amino acid, preferably methionine or arginine. In an embodiment, the CDR-H1 comprises the following sequence: GFTFSSFNMX⁷ (SEQ ID NO:94), wherein X⁷ is any amino acid, preferably phenylalanine, valine, or cysteine.

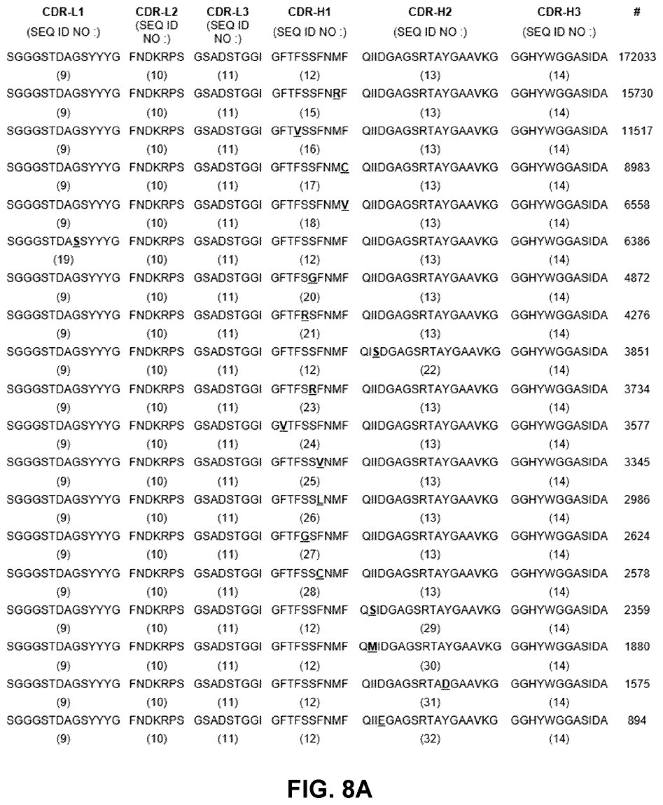

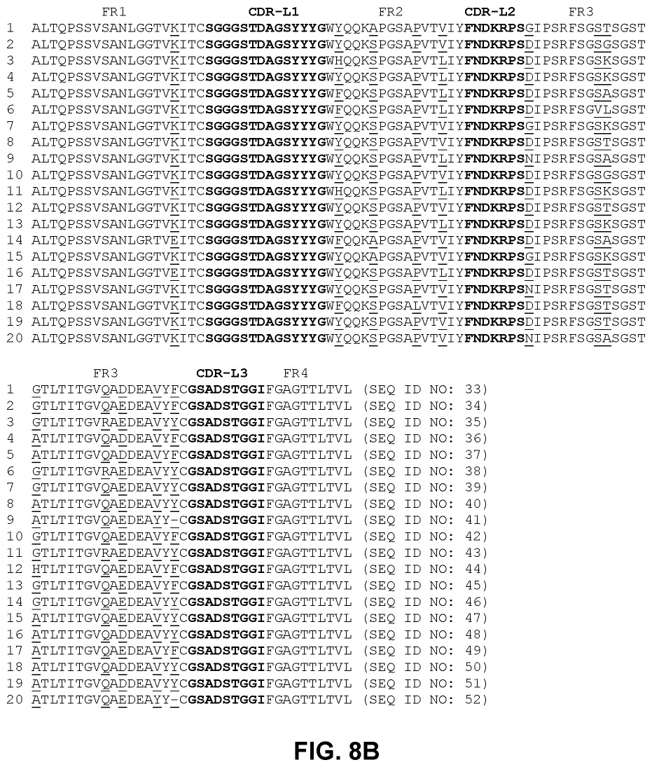

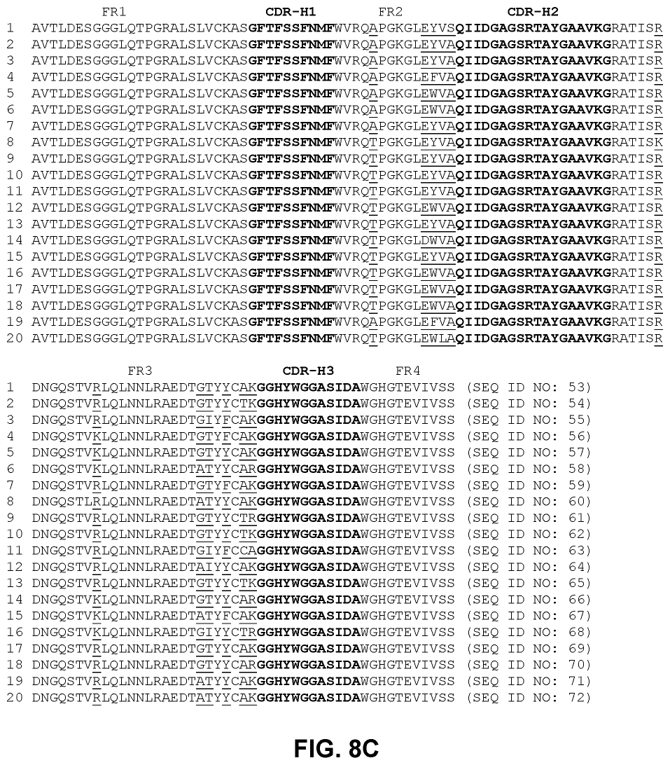

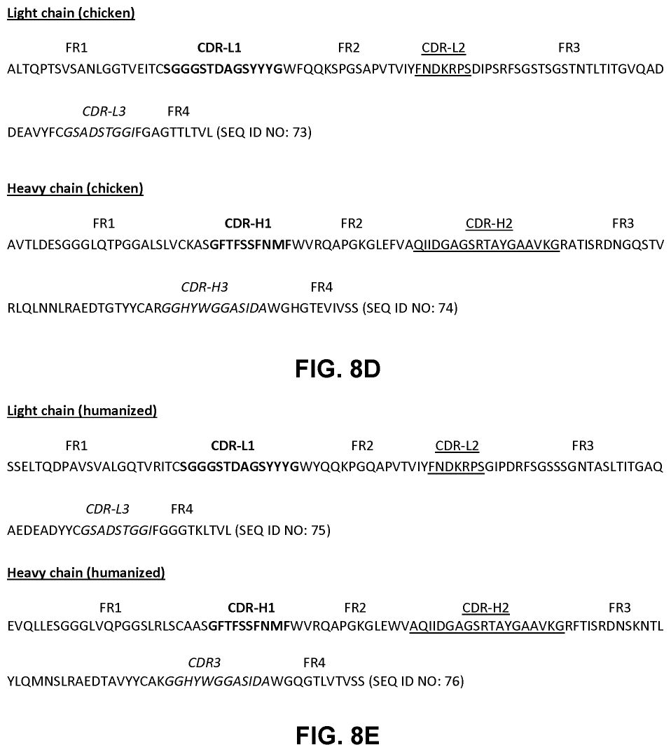

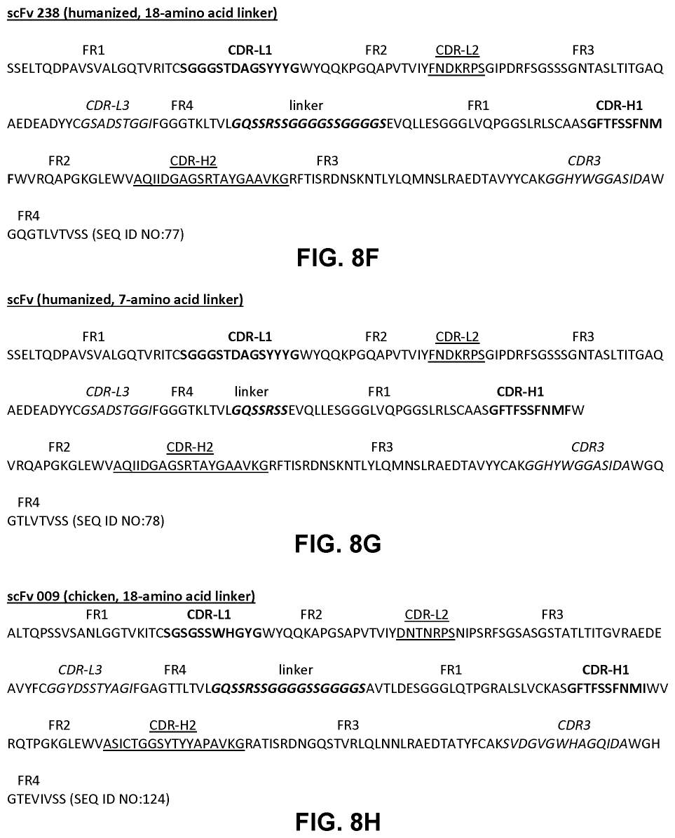

In an embodiment, the CDR-H2 comprises the following sequence: QX⁸X⁹X¹⁰GAGSRTAX¹¹GAAVKG (SEQ ID NO:95), wherein X⁸—X¹¹ are independently any amino acids. In another embodiment, the CDR-H2 comprises the following sequence: QX⁸IDGAGSRTAYGAAVKG (SEQ ID NO:96), wherein X⁸ is any amino acid, preferably isoleucine, serine or methionine. In another embodiment, the CDR-H2 comprises the following sequence: QIX⁹DGAGSRTAYGAAVKG (SEQ ID NO:97), wherein X⁹ is any amino acid, preferably isoleucine or serine. In another embodiment, the CDR-H2 comprises the following sequence: QIIX¹⁰GAGSRTAYGAAVKG (SEQ ID NO:98), wherein X¹⁰ is any amino acid, preferably aspartic or glutamic acid. In another embodiment, the CDR-H2 comprises the following sequence: QIIDGAGSRTAX¹¹GAAVKG (SEQ ID NO:99), wherein X¹¹ is any amino acid, preferably tyrosine or aspartic acid. In an embodiment, the antibody or antigen-binding fragment thereof comprises one or more of the CDRs depicted in FIG. 8A.

In a further embodiment, (i) the CDR-L1 comprises or consists of the sequence SGGGSTDAGSYYYG (SEQ ID NO:9), SGGGSTDASSYYYG (SEQ ID NO:19) or SGSGSSWHGYG (SEQ ID NO: 125), (ii) the CDR-L2 comprises or consists of the sequence FNDKRPS (SEQ ID NO:10) or DNTNRPS (SEQ ID NO:126), (iii) the CDR-L3 comprises or consists of the sequence GSADSTGGI (SEQ ID NO:11) or GGYDSSTYAGI (SEQ ID NO: 127), (iv) the CDR-H1 comprises or consists of the sequence GFTFSSFNMF (SEQ ID NO:12), GFTFSSFNRF (SEQ ID NO:15), GFTVSSFNMF (SEQ ID NO:16), GFTFSSFNMC (SEQ ID NO:17), GFTFSSFNMV (SEQ ID NO:18), GFTFSGFNMF (SEQ ID NO:20), GFTFRSFNMF (SEQ ID NO:21), GFTFSRFNMF (SEQ ID NO:23), GVTFSSFNMF (SEQ ID NO:24), GFTFSSVNMF (SEQ ID NO:25), GFTFSSLNMF (SEQ ID NO:26), GFTFGSFNMF (SEQ ID NO:27), GFTFSSCNMF (SEQ ID NO:28) or GFTFSSFNMI (SEQ ID NO: 128), (v) the CDR-H2 comprises or consists of the sequence QIIDGAGSRTAYGAAVKG (SEQ ID NO:13), QISDGAGSRTAYGAAVKG (SEQ ID NO:22), QSIDGAGSRTAYGAAVKG (SEQ ID NO:29), QMIDGAGSRTAYGAAVKG (SEQ ID NO:30), QIIDGAGSRTADGAAVKG (SEQ ID NO:31), QIIEGAGSRTAYGAAVKG (SEQ ID NO:32) or ASICTGGSYTYYAPAVKG (SEQ ID NO:129), (vi) the CDR-H3 comprises or consists of the sequence GGHYWGGASIDA (SEQ ID NO:14) or SVDGVGWHAGQIDA (SEQ ID NO:130), or (vii) any combination of (i) to (vi).

In a further embodiment, the antibody or antigen-binding fragment thereof comprises the following combination of CDRs: (i) a CDR-L1 comprising or consisting of the sequence SGGGSTDAGSYYYG (SEQ ID NO:9), SGGGSTDASSYYYG (SEQ ID NO:19) or SGSGSSWHGYG (SEQ ID NO: 125), preferably SGGGSTDAGSYYYG (SEQ ID NO:9); (ii) a CDR-L2 comprising or consisting of the sequence FNDKRPS (SEQ ID NO:10) or DNTNRPS (SEQ ID NO:126); (iii) a CDR-L3 comprising or consisting of the sequence GSADSTGGI (SEQ ID NO:11) or GGYDSSTYAGI (SEQ ID NO: 127); (iv) a CDR-H1 comprising or consisting of the sequence GFTFSSFNMF (SEQ ID NO:12), GFTFSSFNRF (SEQ ID NO:15), GFTVSSFNMF (SEQ ID NO:16), GFTFSSFNMC (SEQ ID NO:17), GFTFSSFNMV (SEQ ID NO:18), GFTFSGFNMF (SEQ ID NO:20), GFTFRSFNMF (SEQ ID NO:21), GFTFSRFNMF (SEQ ID NO:23), GVTFSSFNMF (SEQ ID NO:24), GFTFSSVNMF (SEQ ID NO:25), GFTFSSLNMF (SEQ ID NO:26), GFTFGSFNMF (SEQ ID NO:27), GFTFSSCNMF (SEQ ID NO:28) or GFTFSSFNMI (SEQ ID NO: 128), preferably GFTFSSFNMF (SEQ ID NO:12); (v) a CDR-H2 comprising or consisting of the sequence QIIDGAGSRTAYGAAVKG (SEQ ID NO:13), QISDGAGSRTAYGAAVKG (SEQ ID NO:22), QSIDGAGSRTAYGAAVKG (SEQ ID NO:29), QMIDGAGSRTAYGAAVKG (SEQ ID NO:30), QIIDGAGSRTADGAAVKG (SEQ ID NO:31), QIIEGAGSRTAYGAAVKG (SEQ ID NO:32) or ASICTGGSYTYYAPAVKG (SEQ ID NO:129), preferably QIIDGAGSRTAYGAAVKG (SEQ ID NO:13); and (vi) a CDR-H3 comprising or consisting of the sequence GGHYWGGASIDA (SEQ ID NO:14) or SVDGVGWHAGQIDA (SEQ ID NO:130).

In an embodiment, the antibody or antigen-binding fragment thereof comprises: (i) a light chain FR1 comprising or consisting of an amino acid sequence having at least 50%, 60%, 70%, 75%, 80%, 85%, 90% or 95% identity with the sequence ALTQPTSVSANLGGTVEITC (SEQ ID NO:100) or ALTQPSSVSANLGGTVKITC (SEQ ID NO:131); (ii) a light chain FR2 comprising or consisting of an amino acid sequence having at least 50%, 60%, 70%, 75%, 80%, 85%, 90% or 95% identity with the sequence WFQQKSPGSAPVTVIY (SEQ ID NO:101) or WYQQKAPGSAPVTVIY (SEQ ID NO:132); (iii) a light chain FR3 comprising or consisting of an amino acid sequence having at least 50%, 60%, 70%, 75%, 80%, 85%, 90% or 95% identity with the sequence DIPSRFSGSTSGSTNTLTITGVQADDEAVYFC (SEQ ID NO:102) or NIPSRFSGSASGSTATLTITGVRAEDEAVYFC (SEQ ID NO:133); (iv) a light chain FR4 comprising or consisting of an amino acid sequence having at least 50%, 60%, 70%, 75%, 80%, 85%, 90% or 95% identity with the sequence FGAGTTLTVL (SEQ ID NO:103) or FGAGTTLTVL (SEQ ID NO:134); or (v) any combination of (i) to (iv). In an embodiment, the light chain FR1 comprises or consists of one of the FR1 amino acid sequences depicted in FIG. 8B. In an embodiment, the light chain FR2 comprises or consists of one of the FR2 amino acid sequences depicted in FIG. 8B. In an embodiment, the light chain FR3 comprises or consists of one of the FR3 amino acid sequences depicted in FIG. 8B. In an embodiment, the light chain FR4 comprises or consists of one of the FR4 amino acid sequences depicted in FIG. 8B.

In a further embodiment, the antibody or antigen-binding fragment thereof comprises: (i) a light chain FR1 comprising or consisting of the sequence ALTQPTSVSANLGGTVEITC (SEQ ID NO:100) or ALTQPSSVSANLGGTVKITC (SEQ ID NO:131); (ii) a light chain FR2 comprising or consisting of the sequence WFQQKSPGSAPVTVIY (SEQ ID NO:101) or WYQQKAPGSAPVTVIY (SEQ ID NO:132); (iii) a light chain FR3 comprising or consisting of the sequence DIPSRFSGSTSGSTNTLTITGVQADDEAVYFC (SEQ ID NO:102) or NIPSRFSGSASGSTATLTITGVRAEDEAVYFC (SEQ ID NO:133); (iv) a light chain FR4 comprising or consisting of the sequence FGAGTTLTVL (SEQ ID NO:103) or FGAGTTLTVL (SEQ ID NO:134); or (v) any combination of (i) to (iv). In a further embodiment, the antibody or antigen-binding fragment thereof comprises features (i) to (iv).

In an embodiment, the antibody or antigen-binding fragment thereof comprises: (i) a heavy chain FR1 comprising or consisting of an amino acid sequence having at least 50%, 60%, 70%, 75%, 80%, 85%, 90% or 95% identity with the sequence AVTLDESGGGLQTPGGALSLVCKAS (SEQ ID NO:104) or AVTLDESGGGLQTPGRALSLVCKAS (SEQ ID NO:135); (ii) a heavy chain FR2 comprising or consisting of an amino acid sequence having at least 50%, 60%, 70%, 75%, 80%, 85%, 90% or 95% identity with the sequence WVRQAPGKGLEFV (SEQ ID NO:105) or WVRQTPGKGLEWV (SEQ ID NO:136); (iii) a heavy chain FR3 comprising or consisting of an amino acid sequence having at least 50%, 60%, 70%, 75%, 80%, 85%, 90% or 95% identity with the sequence RATISRDNGQSTVRLQLNNLRAEDTGTYYCAR (SEQ ID NO:106) or RATISRDNGQSTVRLQLNNLRAEDTATYFCAK (SEQ ID NO:137); (iv) a heavy chain FR4 comprising or consisting of an amino acid sequence having at least 50%, 60%, 70%, 75%, 80%, 85%, 90% or 95% identity with the sequence WGHGTEVIVSS (SEQ ID NO:107) or WGHGTEVIVSS (SEQ ID NO:138); or (v) any combination of (i) to (iv). In an embodiment, the heavy chain FR1 comprises or consists of one of the FR1 amino acid sequences depicted in FIG. 8C. In an embodiment, the heavy chain FR2 comprises or consists of one of the FR2 amino acid sequences depicted in FIG. 8C. In an embodiment, the heavy chain FR3 comprises or consists of one of the FR3 amino acid sequences depicted in FIG. 8C. In an embodiment, the heavy chain FR4 comprises or consists of one of the FR4 amino acid sequences depicted in FIG. 8C.

In an embodiment, the antibody or antigen-binding fragment thereof comprises: (i) a heavy chain FR1 comprising or consisting of the sequence SEQ ID NO:104 or 135; (ii) a heavy chain FR2 comprising or consisting of the sequence SEQ ID NO:105 or 136; (iii) a heavy chain FR3 comprising or consisting of the sequence SEQ ID NO:106 or 137; (iv) a heavy chain FR4 comprising or consisting of the sequence SEQ ID NO:107 or 138; or (v) any combination of (i) to (iv). In a further embodiment, the antibody or antigen-binding fragment thereof comprises features (i) to (iv).

In an embodiment, the antibody or antigen-binding fragment thereof comprises a variable light chain comprising or consisting of an amino acid sequence having at least 70%, 75%, 80%, 85%, 90% or 95% identity with the sequence

(SEQ ID NO: 108)
ALTQPTSVSANLGGTVEITCSGGGSTDAGSYYYGWFQQKSPGSAPVTVI
YFNDKRPSDIPSRFSGSTSGSTNTLTITGVQADDEAVYFCGSADSTGGI
FGAGTTLTVL
or
(SEQ ID NO: 139)
ALTQPSSVSANLGGTVKITCSGSGSSWHGYGWYQQKAPGSAPVTVIYDN
TNRPSNIPSRFSSASGSTATLTITGVRAEDEAVYFCGGYDSSTYAGIFG
AGTTLTVL.

In an embodiment, the differences relative to the reference variable light chain sequence are within one or more of the FRs underlined above. In a further embodiment, the antibody or antigen-binding fragment thereof comprises a variable light chain comprising or consisting of the sequence of SEQ ID NO:108 or 139.

In an embodiment, the antibody or antigen-binding fragment thereof comprises a variable heavy chain comprising or consisting of an amino acid sequence having at least 70%, 75%, 80%, 85%, 90% or 95% identity with the sequence

(SEQ ID NO: 109)
AVTLDESGGGLQTPGGALSLVCKASGFTFSSFNMFWVRQAPGKGLEFVA
QIIDGAGSRTAYGAAVKGRATISRDNGQSTVRLQLNNLRAEDTGTYYCA
RGGHYWGGASIDAWGHGTEVIVSS
or
(SEQ ID NO: 140)
AVTLDESGGGLQTPGRALSLVCKASGFTFSSFNMIWVRQTPGKGLEWVA
SICTGGSYTYYAPAVKGRATISRDNGQSTVRLQLNNLRAEDTATYFCAK
SVDGVGWHAGQIDAWGHGTEVIVSS.

In an embodiment, the differences relative to the reference variable heavy chain sequence are within one or more of the FRs underlined above. In a further embodiment, the antibody or antigen-binding fragment thereof comprises a variable heavy chain comprising or consisting of the sequence of SEQ ID NO:109 or 140.

The sequences of the CDRs and FRs described herein are based on the numbering of Chothia (Al-Lazikani et al., J Mol Biol. 1997 Nov. 7; 273(4):927-48).

In an embodiment, the antibody or antigen-binding fragment thereof is a humanized form of the above-noted antibody or antigen-binding fragment thereof.

In another aspect, the present disclosure relates to antibody or antigen-binding fragment thereof, preferably a chimeric or humanized antibody or antigen-binding fragment, comprising any of the combinations of CDRs disclosed herein.

“Humanized” forms of non-human (e.g., chicken, rodent) antibodies are chimeric antibodies that contain minimal sequence derived from non-human immunoglobulin. For the most part, humanized antibodies are human immunoglobulins (recipient antibody) in which residues from a hypervariable region (e.g., a CDR) of the recipient are replaced by residues from a hypervariable region of a non-human species (donor antibody) such as chicken, mouse, rat, rabbit or nonhuman primate having the desired specificity, affinity, and capacity, such as the CDRs defined herein. In some instances, FR residues of the human immunoglobulin are replaced by corresponding non-human residues. Furthermore, humanized antibodies may comprise residues that are not found in the recipient antibody or in the donor antibody. Humanized antibodies may also correspond to non-human (e.g., chicken) immunoglobulins having the desired specificity, affinity, and capacity in which residues from one or more of the FRs are replaced by residues from one or more FRs of a human immunoglobulin. These modifications are made to further refine antibody performance. In general, the humanized antibody will comprise substantially all of at least one, and typically two, variable domains, in which all or substantially all of the hypervariable loops correspond to those of a non-human (e.g., chicken) immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Opin. Struct. Biol. 2:593-596 (1992).

Human chimeric antibody and human CDR-grafted antibody may be prepared using methods known in the art. A representative method is described below.

(1) Construction of Vector for Recombinant Antibody Expression. A vector for recombinant antibody expression is an expression vector for animal cell into which DNAs encoding C_{H} and C_{L} of a human antibody have been inserted, and is constructed by cloning each of DNAs encoding C_{H} and C_{L} of a human antibody into an expression vector for animal cell. The constant region (hereinafter, referred to as C region) of a human antibody may be C_{H} and C_{L} of any human antibody. Examples include C_{H} of γl subclass and C_{L} of K class of human antibody, or the like. As the DNAs encoding C_{H} and C_{L} of a human antibody, the cDNA may be generally used and a chromosomal DNA composed of an exon and an intron can be also used. As the expression vector for animal cell, any expression vector can be used, as long as a gene encoding the C region of a human antibody can be inserted thereinto and expressed therein. Examples thereof include pAGE107 [Cytotechnol., 3, 133 (1990)], pAGE103 [J. Biochem., 101, 1307 (1987)], pHSG274 [Gene, 27, 223 (1984)], pKCR [Proc. Natl. Acad. Sci. USA, 78, 1527 (1981)], pSG1bd2-4 [Cytotechnol., 4, 173 (1990)], pSE1UK1Sed1-3 [Cytotechnol., 13, 79 (1993)] or the like. Examples of a promoter and an enhancer used for an expression vector for animal cell include an SV40 early promoter [J. Biochem., 101, 1307 (1987)], a Moloney mouse leukemia virus LTR [Biochem. Biophys. Res. Commun., 149, 960 (1987)], an immunoglobulin H chain promoter [Cell, 41, 479 (1985)], an enhancer [Cell, 33, 717 (1983)] or the like. As the vector for recombinant antibody expression, a type of the vector for recombinant antibody expression in which both of antibody H and L chains exist on the same vector (tandem type) (J. Immunol. Methods, 167, 271 (1994)) may be used, in terms of easiness of construction of a vector for recombinant antibody expression, easiness of introduction into animal cells, and balance between the expression levels of antibody H and L chains in animal cells, and a type in which antibody H and L chains exist on separate vectors may be also used. Examples of the tandem type of the vector for recombinant antibody expression include pKANTEX93 (WO 97/10354), pEE18 (Hybridoma, 17, 559 (1998)), or the like.

(2) Acquisition of cDNA Encoding V Region of Antibody Derived from Non-Human Animal and Analysis of Amino Acid Sequence. mRNA is extracted from hybridoma cells producing a non-human antibody (e.g., chicken antibody) to synthesize cDNA. The synthesized cDNA is cloned into a vector such as a phage or a plasmid, to prepare a cDNA library. Each of a recombinant phage or recombinant plasmid containing cDNA encoding V_{H} or V_{L} is isolated from the library using DNA encoding the C region or V region of a non-human antibody as the probe. The full length of the base sequences of V_{H} and V_{L} of a non-human antibody of interest on the recombinant phage or recombinant plasmid are determined, and the full length of the amino acid sequences of V_{H} and V_{L} are deduced from the base sequences, respectively. Examples of the non-human animal for preparing a hybridoma cell which produces a non-human antibody include chicken, mouse, rat, hamster, rabbit or the like. Any animals can be used as long as a hybridoma cell can be produced therefrom. Total RNA can be prepared from a hybridoma cell using a guanidine thiocyanate-cesium trifluoroacetate method (Methods in Enzymol., 154, 3 (1987)), or a kit such as RNA easy kit (manufactured by Qiagen®) or the like. mRNA can be prepared from total RNA using an oligo (dT) immobilized cellulose column method (Molecular Cloning, A Laboratory Manual, Second Edition, Cold Spring Harbor Laboratory Press (1989)), a method using a kit such as Oligotex™-dT30<Super> mRNA Purification Kit (manufactured by Takara Bio) or the like. In addition, mRNA can be prepared from hybridoma cells using a kit such as a Fast Track® mRNA Isolation kit (manufactured by Invitrogen®), a QuickPrep® mRNA Purification Kit (manufactured by Pharmacia®) or the like. Examples of the method for synthesizing cDNA and preparing a cDNA library include known methods (Molecular Cloning, A Laboratory Manual, Second Edition, Cold Spring Harbor Laboratory Press (1989), Current Protocols in Molecular Biology, Supplement 1, John Wiley & Sons (1987-1997)), a method using a kit such as a Super Script® Plasmid System for cDNA Synthesis and Plasmid Cloning (manufactured by Invitrogen®), a ZAP-cDNA Synthesis Kit (manufactured by Stratagene®), or the like. The vector for preparing a cDNA library, into which cDNA synthesized using mRNA extracted from a hybridoma cell as the template is inserted, may be any vector, as long as the cDNA can be inserted thereto. Examples thereof include ZAP ExPress (Strategies, 5, 58 (1992)), pBluescript II SK(+) [Nucleic Acids Research, 17, 9494 (1989)], λZAPII (manufactured by Stratagene®), λgt10 and λgt11 (DNA Cloning: A Practical Approach, I, 49 (1985)), Lambda BlueMid (manufactured by Clontech®), AExCell, pT7T3-18U (manufactured by Pharmacia®), pcD2 (Mol. Cell. Biol., 3, 280 (1983)), pUC18 (Gene, 33, 103 (1985)), or the like. Any Escherichia coli for introducing the cDNA library constructed by a phage or plasmid vector may be used, as long as the cDNA library can be introduced, expressed and maintained. Examples thereof include XL1-Blue MRF (Strategies, 5, 81 (1992)), C600 (Genetics, 39, 440 (1954)), Y1088 and Y1090 (Science, 222: 778 (1983)), NM522 (J. Mol. Biol., 166, 1 (1983)), K802 (J. Mol. Biol., 16, 118 (1966)), JM105 (Gene, 38, 275 (1985)), or the like. A colony hybridization or plaque hybridization method using an isotope- or fluorescence-labeled probe (Molecular Cloning, A Laboratory Manual, Second Edition, Cold Spring Harbor Laboratory Press (1989)) may be used for selecting cDNA clones encoding V_{H} or V_{L} of a non-human antibody or the like from the cDNA library. Also, the cDNA encoding V_{H} or V_{L} can be prepared through polymerase chain reaction by preparing primers and using cDNA prepared from mRNA or a cDNA library as the template. The base sequence of the cDNA can be determined by digesting the cDNA selected with appropriate restriction enzymes or the like, cloning the fragments into a plasmid such as pBluescript SK(−), carrying out a sequence analyzing method usually used. For example, the sequence analyzing method is carried out by using an automatic nucleotide sequence analyzer such as ABI PRISM37000 (manufactured by PE Biosystems®) or A.L.F. DNA sequencer (manufactured by Pharmacia®) after reaction such as the dideoxy method (Proc. Natl. Acad. Sci. USA, 74, 5463 (1977)). Whether the obtained cDNAs encode the full amino acid sequences of V_{H} and V_{L} of the antibody containing a secretory signal sequence can be confirmed by estimating the full length of the amino acid sequences of V_{H} and V_{L} from the determined nucleotide sequence and comparing them with the full length of the amino acid sequences of V_{H} and V_{L} of known antibodies (A.L.F. DNA sequencer, US Dept. Health and Human Services (1991)), and furthermore the subgroup to which they belong can be determined. In addition, the amino acid sequence of each CDR of V_{H} and V_{L} can be determined by comparing them with the amino acid sequences of V_{H} and V_{L} of known antibodies.

(3) Construction of Vector for Human Chimeric Antibody Expression. cDNA encoding each of V_{H} and V_{L} of antibody of non-human animal (e.g., chicken) is cloned in the upstream of genes encoding C_{H} or C_{L} of human antibody of vector for expression of recombinant antibody mentioned above, thereby constructing a vector for human chimeric antibody expression. In order to ligate the 3′-terminus of cDNA encoding V_{H} or V_{L} of antibody of non-human animal and the 5′-terminus of C_{H} or C_{L} of human antibody, each cDNA encoding V_{H} and V_{L} is prepared so as to encodes appropriate amino acids encoded by a base sequence of a linkage portion and designed to have an appropriate recognition sequence of a restriction enzyme. The prepared cDNAs of V_{H} and V_{L} are respectively cloned so that each of them is expressed in an appropriate form in the upstream of gene encoding C_{H} or C_{L} of the human antibody of the vector for the human CDR-grafted antibody expression mentioned in the above (1) to construct a vector for human chimeric antibody expression. In addition, cDNA encoding V_{H} or V_{L} of a non-human animal antibody is amplified by PCR using a synthetic DNA having a recognition sequence of an appropriate restriction enzyme at both ends, and each of them is cloned to the vector obtained in the above (1) for recombinant antibody expression.

(4) Construction of cDNA Encoding V Region of Human CDR-Grafted Antibody. Amino acid sequences of FR in V_{H} or V_{L} of a human antibody, to which amino acid sequences of CDRs in V_{H} or V_{L} of a non-human antibody (e.g., the CDRs of the antibodies described herein) are grafted, are selected, respectively. Any amino acid sequences of FR can be used, as long as they are derived from human. Examples thereof include amino acid sequences of FRs of human antibodies registered in database such as Protein Data Bank or the like, or amino acid sequences common to subgroups of FRs of human antibodies [A. L. F. DNA, US Dept. Health and Human Services (1991)] or the like. In order to inhibit the decrease in the binding activity of the antibody, amino acid sequences of FR having high homology (at least 60% or more) with the amino acid sequence of FR in V_{H} or V_{L} of the original antibody is selected. Then, amino acid sequences of CDRs of the original antibody are grafted to the selected amino acid sequence of FR in V_{H} or V_{L} of the human antibody, respectively, to design each amino acid sequence of V_{H} or V_{L} of a human CDR-grafted antibody. The designed amino acid sequences are converted to DNA sequences by considering the frequency of codon usage found in nucleotide sequences of genes of antibodies, and the DNA sequence encoding the amino acid sequence of V_{H} or V_{L} of a human CDR-grafted antibody is designed. Based on the designed DNA sequences, several synthetic DNAs having a length of about 100 nucleotides are synthesized, and PCR is carried out using them. In this case, it is preferred that 6 synthetic DNAs per each of the H chain and the L chain are designed in view of the reaction efficiency of PCR and the lengths of DNAs which can be synthesized. Furthermore, the cDNA encoding V_{H} or V_{L} of a human CDR-grafted antibody can be easily cloned into the vector for expressing the human CDR-grafted antibody constructed in (1) by introducing the recognition sequence of an appropriate restriction enzyme to the 5′ terminal of the synthetic DNAs existing on the both ends. Otherwise, it can be carried out using a synthetic DNA as a single DNA encoding each of the full-length H chain and the full-length L chain based on the designed DNA sequence. After PCR, an amplified product is cloned into a plasmid such as pBluescript SK (−) or the like, and the base sequence is determined according to a method similar to the method described in (2) to obtain a plasmid having a DNA sequence encoding the amino acid sequence of V_{H} or V_{L} of a desired human CDR-grafted antibody.

(5) Modification of Amino Acid Sequence of V Region of Human CDR-Grafted Antibody. It is known that when a human CDR-grafted antibody is produced by grafting only CDRs in V_{H} and V_{L} of a non-human antibody into FRs of V_{H} and V_{L} of a human antibody, its antigen binding activity is sometimes lower than that of the original non-human antibody [BIO/TECHNOLOGY, 9, 266 (1991)]. In human CDR-grafted antibodies, among the amino acid sequences of FRs in V_{H} and V_{L} of a human antibody, amino acid residues that are directly involved in the binding to an antigen, amino acid residues that interacts with an amino acid residue in CDR, and amino acid residues that maintain the three-dimensional structure of an antibody and indirectly involved in the binding to an antigen may be identified and replaced with amino acid residues which are found in the original non-human antibody, thereby increasing the antigen binding activity which has been decreased. In order to identify the amino acid residues involved in the antigen binding activity in FR, three-dimensional structure of an antibody can be constructed and analyzed by X-ray crystallography, computer-modeling or the like. In addition, modified human CDR-grafted antibody having sufficient binding activity against antigen can be obtained by producing several modified antibodies of each antibody and examining their antigen binding activities to identify those having improved affinity. The modification of the amino acid sequence of FR in V_{H} and V_{L} of a human antibody can be accomplished using various synthetic DNA for modification according to PCR as described in (4). With regard to the amplified product obtained by PCR, the base sequence is determined according to the method as described in (2) so as to examine whether the desired modification has been carried out.

(6) Construction of Vector for Human CDR-Grafted Antibody Expression. A vector for human CDR-grafted antibody expression can be constructed by cloning each cDNA encoding V_{H} or V_{L} of a constructed recombinant antibody into upstream of each gene encoding C_{H} or C_{L} of the human antibody in the vector for recombinant antibody expression as described in (1). For example, recognizing sequences of an appropriate restriction enzymes are introduced to the 5′-terminal of synthetic DNAs positioned at both ends among synthetic DNAs used in the construction of V_{H} or V_{L} of the human CDR-grafted antibody in (4) and (5), and cloning can be carried out so that they are expressed in an appropriate form in the upstream of each gene encoding C_{H} or C_{L} of the human antibody in the vector for a human CDR-grafted antibody expression as described in (1).

(7) Transient Expression of Recombinant Antibody. The recombinant antibodies can be expressed transiently using the vector for recombinant antibody expression obtained in (3) and (6) or the modified expression vector thereof so as to efficiently evaluate the antigen binding activity of various human CDR-grafted antibodies. Any cell can be used as a host cell, as long as the host cell is able to express a recombinant antibody (e.g., CHO cells, COS cells). For example, COS-7 cell (ATCC CRL1651) is used. Introduction of the expression vector into COS-7 cell is performed by using a DEAE-dextran method, a lipofection method, or the like. After introduction of the expression vector, the expression level and antigen binding activity of the recombinant antibody in the culture supernatant can be determined by the enzyme immunoassay or the like.

(8) Acquisition of Transformant which Stably Expresses Recombinant Antibody and Preparation of Recombinant Antibody. A transformant which stably expresses a recombinant antibody can be obtained by introducing the vector for recombinant antibody expression obtained in (3) and (6) into an appropriate host cell. Introduction of the expression vector into a host cell is performed by electroporation or the like. As the host cell into which a vector for recombinant antibody expression is introduced, any cell can be used, as long as it is a host cell which is able to produce the recombinant antibody. Examples thereof include CHO-K1 (ATCC CCL-61), DUkXB11 (ATCC CCL-9096), Pro-5 (ATCC CCL-1781), CHO-S (Life Technologies®, Cat #11619), rat myeloma cell YB2/3HL.P2.G11.16Ag.20 (also called YB2/0), mouse myeloma cell NSO, mouse myeloma cell SP2/0-Ag14 (ATCC No. CRL1581), mouse P3-X63-Ag8653 cell (ATCC No. CRL1580), CHO cell in which a dihydrofolate reductase gene is defective, lectin resistance-acquired Lec13, CHO cell in which a1,6-fucosyltransaferse gene is defective, rat YB2/3HL.P2.G11.16Ag.20 cell (ATCC No. CRL1662), CHO-3E7 cells (expressing a truncated but functional form of EBNA1, U.S. Pat. No. 8,637,315) or the like. After introduction of the expression vector, transformants which stably express a recombinant antibody are selected by culturing them in a medium for animal cell culture containing an agent such as G418 sulfate or the like. Examples of the medium for animal cell culture include RPM11640 medium (manufactured by Invitrogen®), GIT medium (manufactured by Nihon Pharmaceutical®), EX-CELL301® medium (manufactured by JRH®), IMDM medium (manufactured by Invitrogen®), Hybridoma-SFM medium (manufactured by Invitrogen®), media obtained by adding various additives such as FBS to these media, or the like. The recombinant antibody can be produced and accumulated in a culture supernatant by culturing the obtained transformants in a medium. The expression level and antigen binding activity of the recombinant antibody in the culture supernatant can be measured by ELISA or the like. Also, in the transformant, the expression level of the recombinant antibody can be increased by using DHFR amplification system or the like. The recombinant antibody can be purified from the culture supernatant of the transformant by using a protein A column. In addition, the recombinant antibody can be purified by combining the protein purification methods such as gel filtration, ion-exchange chromatography, ultrafiltration or the like. The molecular weight of the H chain or the L chain of the purified recombinant antibody or the antibody molecule as a whole is determined by polyacrylamide gel electrophoresis, Western blotting, or the like.

In an embodiment, the humanized antibody or antigen-binding fragment thereof comprises: (i) a light chain FR1 comprising or consisting of an amino acid sequence having at least 50%, 60%, 70%, 75%, 80%, 85%, 90% or 95% identity with the sequence SSELTQDPAVSVALGQTVRITC (SEQ ID NO:110); (ii) a light chain FR2 comprising or consisting of an amino acid sequence having at least 50%, 60%, 70%, 75%, 80%, 85%, 90% or 95% identity with the sequence WYQQKPGQAPVTVIY (SEQ ID NO:111); (iii) a light chain FR3 comprising or consisting of an amino acid sequence having at least 50%, 60%, 70%, 75%, 80%, 85%, 90% or 95% identity with the sequence GIPDRFSGSSSGNTASLTITGAQAEDEADYYC (SEQ ID NO:112); (iv) a light chain FR4 comprising or consisting of an amino acid sequence having at least 50%, 60%, 70%, 75%, 80%, 85%, 90% or 95% identity with the sequence FGGGTKLTVL (SEQ ID NO:113); or (v) any combination of (i) to (iv).

In a further embodiment, the humanized antibody or antigen-binding fragment thereof comprises: (i) a light chain FR1 comprising or consisting of the sequence SSELTQDPAVSVALGQTVRITC (SEQ ID NO:110); (ii) a light chain FR2 comprising or consisting of the sequence WYQQKPGQAPVTVIY (SEQ ID NO:111); (iii) a light chain FR3 comprising or consisting of the sequence GIPDRFSGSSSGNTASLTITGAQAEDEADYYC (SEQ ID NO:112); (iv) a light chain FR4 comprising or consisting of the sequence FGGGTKLTVL (SEQ ID NO:113); or (v) any combination of (i) to (iv). In a further embodiment, the humanized antibody or antigen-binding fragment thereof comprises features (i) to (iv).

In an embodiment, the humanized antibody or antigen-binding fragment thereof comprises: (i) a heavy chain FR1 comprising or consisting of an amino acid sequence having at least 50%, 60%, 70%, 75%, 80%, 85%, 90% or 95% identity with the sequence EVQLLESGGGLVQPGGSLRLSCAAS (SEQ ID NO:114); (ii) a heavy chain FR2 comprising or consisting of an amino acid sequence having at least 50%, 60%, 70%, 75%, 80%, 85%, 90% or 95% identity with the sequence WVRQAPGKGLEWV (SEQ ID NO:115); (iii) a heavy chain FR3 comprising or consisting of an amino acid sequence having at least 50%, 60%, 70%, 75%, 80%, 85%, 90% or 95% identity with the sequence RFTISRDNSKNTLYLQMNSLRAEDTAVYYCAK (SEQ ID NO:116); (iv) a heavy chain FR4 comprising or consisting of an amino acid sequence having at least 50%, 60%, 70%, 75%, 80%, 85%, 90% or 95% identity with the sequence WGQGTLVTVSS (SEQ ID NO:117); or (v) any combination of (i) to (iv).

In an embodiment, the humanized antibody or antigen-binding fragment thereof comprises: (i) a heavy chain FR1 comprising or consisting of the sequence EVQLLESGGGLVQPGGSLRLSCAAS (SEQ ID NO:114); (ii) a heavy chain FR2 comprising or consisting of the sequence WVRQAPGKGLEWV (SEQ ID NO:115); (iii) a heavy chain FR3 comprising or consisting of the sequence RFTISRDNSKNTLYLQMNSLRAEDTAVYYCAK (SEQ ID NO:116); (iv) a heavy chain FR4 comprising or consisting of the sequence WGQGTLVTVSS (SEQ ID NO:117); or (v) any combination of (i) to (iv). In a further embodiment, the humanized antibody or antigen-binding fragment thereof comprises features (i) to (iv).

In an embodiment, the humanized antibody or antigen-binding fragment thereof comprises a variable light chain comprising or consisting of an amino acid sequence having at least 70%, 75%, 80%, 85%, 90% or 95% identity with the sequence

(SEQ ID NO: 118)
SSELTQDPAVSVALGQTVRITCSGGGSTDAGSYYYGWYQQKPGQAPVTV
IYFNDKRPSGIPDRFSGSSSGNTASLTITGAQAEDEADYYCGSADSTGG
IFGGGTKLTVL.

In another aspect, the present disclosure relates to a humanized antibody or antigen-binding fragment thereof comprising a variable light chain comprising or consisting of an amino acid sequence having at least 70%, 75%, 80%, 85%, 90% or 95% identity with the sequence

(SEQ ID NO: 118)
SSELTQDPAVSVALGQTVRITCSGGGSTDAGSYYYGWYQQKPGQAPVTV
IYFNDKRPSGIPDRFSGSSSGNTASLTITGAQAEDEADYYCGSADSTGG
IFGGGTKLTVL.

In an embodiment, the differences relative to the reference variable light chain sequence are within one or more of the FRs underlined above. In a further embodiment, the humanized antibody or antigen-binding fragment thereof comprises a variable light chain comprising or consisting of the sequence

(SEQ ID NO: 118)
SSELTQDPAVSVALGQTVRITCSGGGSTDAGSYYYGWYQQKPGQAPVTV
IYFNDKRPSGIPDRFSGSSSGNTASLTITGAQAEDEADYYCGSADSTGG
IFGGGTKLTVL.

In an embodiment, the humanized antibody or antigen-binding fragment thereof comprises a variable heavy chain comprising or consisting of an amino acid sequence having at least 70%, 75%, 80%, 85%, 90% or 95% identity with the sequence

(SEQ ID NO: 119)
EVQLLESGGGLVQPGGSLRLSCAASGFTFSSFNMFWVRQAPGKGLEWVA
QIIDGAGSRTAYGAAVKGRFTISRDNSKNTLYLQMNSLRAEDTAVYYCA
KGGHYWGGASIDAWGQGTLVTVSS.

In another aspect, the present disclosure relates to a humanized antibody or antigen-binding fragment thereof comprising a variable heavy chain comprising or consisting of an amino acid sequence having at least 70%, 75%, 80%, 85%, 90% or 95% identity with the sequence

EVQLLESGGGLVQPGGSLRLSCAASGFTFSSFNMFWVRQAPGKGLEWVA
QIIDGAGSRTAYGAAVKGRFTISRDNSKNTLYLQMNSLRAEDTAVYYCA
KGGHYWGGASIDAWGQGTLVTVSS.
or

In an embodiment, the differences relative to the reference variable heavy chain sequence are within one or more of the FRs underlined above. In a further embodiment, the humanized antibody or antigen-binding fragment thereof comprises a variable heavy chain comprising or consisting of the sequence:

EVQLLESGGGLVQPGGSLRLSCAASGFTFSSFNMFWVRQAPGKGLEWVA
QIIDGAGSRTAYGAAVKGRFTISRDNSKNTLYLQMNSLRAEDTAVYYCA
KGGHYWGGASIDAWGQGTLVTVSS.
or

In another embodiment, the anti-NTSR1 antibody or antigen-binding fragment thereof is a single-chain antibody, preferably a single-chain Fv (scFv). In an embodiment, the scFv comprises the CDRs, FRs and/or variable regions defined above, and wherein the light chain variable region (V_{L}) and heavy chain variable region (V_{H}) are linked or connected together through a linker. The linker may connect the N-terminus of the V_{H} with the C-terminus of the V_{L}, or the N-terminus of the V_{L} with the C-terminus of the V_{H}. In an embodiment, the linker connects the N-terminus of the V_{H} with the C-terminus of the V_{L}.

The linker may be a polypeptide linker comprising one or more amino acids or another type of chemical linker (e.g., a carbohydrate linker, a lipid linker, a fatty acid linker, a polyether linker, PEG, etc. having suitable flexibility and stability to allow the light and heavy chains to adopt a proper conformation for antigen recognition (i.e. to maintain the ability to bind the above-noted conformational epitope from NTSR1) In an embodiment, the linker is a polypeptide linker. In an embodiment, the polypeptide linker comprises at least 2, 3, 4 or 5 amino acids. In an embodiment, the polypeptide linker comprises about 100, 90, 80, 70, 60 or 50 amino acids or less. In a further embodiment, the polypeptide linker comprises about 5 to about 50 amino acids, for example about 5 to about 40 amino acids or about 7 to about 30 amino acids, for example about 7 to about 20 or 25 amino acids. In a further embodiment, the linker comprises about 15 to about 20 amino acids or about 18 to 20 amino acids, preferably 18 amino acids. The polypeptide linker is designed to have suitable flexibility and stability to allow the light and heavy chains to adopt a proper conformation for antigen recognition (i.e. to maintain the ability to bind the above-noted conformational epitope from NTSR1). Methods for designing flexible polypeptide linkers, and more specifically linkers with minimal globularity and maximal disorder, suitable for scFv fragments are known in the art. This may be achieved, for example, using the Globplot 2.3 program or any other suitable tool. The sequence may be further optimized to eliminate putative aggregation hotspots, localization domains, and/or interaction and phosphorylation motifs. In an embodiment, the polypeptide linker is enriched in glycine residues, i.e. wherein at least 20%, 25%, 30%, 35%, 40%, 45% or 50% of the residues of the linker are glycine residues (which are known to favor linker flexibility). In an embodiment, the polypeptide linker comprises one or more serine (Ser or S) and/or threonine residues (which are known to favor linker solubility), preferably serine residues. In another embodiment, the polypeptide linker comprises the pentapeptide sequence GGGGS (or G4S or Gly4Ser). In an embodiment, the polypeptide linker comprises at least one arginine residue. In another embodiment, the polypeptide linker comprises at least one glutamine residue. In a further embodiment, the polypeptide linker comprises the sequence GQSSRSS (SEQ ID NO:120). In yet a further embodiment, the polypeptide linker comprises or consists of the sequence GQSSRSSGGGGSSGGGGS (SEQ ID NO:121).

In an embodiment, the anti-NTSR1 antibody or antigen-binding fragment thereof comprises at least one constant domain, e.g., a constant domain of a light and/or heavy chain, or a fragment thereof. In a further embodiment, the anti-NTSR1 antibody or antigen-binding fragment thereof comprises a Fragment crystallizable (Fc) fragment of the constant heavy chain of an antibody. The Fc fragment may comprise two or three constant domains, e.g., a CH₂ domain and CH₃ domain. The Fc region may be obtained from a human IgG1, a human IgG4, or a variant of a human IgG1 or IgG4 having up to ten amino acid modifications, for example. In an embodiment, the Fc fragment comprises or consists of the CH₂ domain and CH₃ domain of a human antibody, preferably a human IgG such as IgG1. In an embodiment, the Fc fragment comprises a sequence having at least 50%, 60%, 70%, 75%, 80%, 85%, 90% or 95% identity with the sequence in bold in FIG. 8G (residues 14-233 of SEQ ID NO: 79). In an embodiment, the Fc fragment comprises or consists of the sequence in bold in FIG. 8G.

In an embodiment, the anti-NTSR1 antibody or antigen-binding fragment is a scFv comprising a Fc fragment (scFV-Fc), such as the Fc fragment defined above. In an embodiment, the scFv component is connected to the Fc fragment by a linker, for example a hinge. The hinge may comprise amino acid sequences derived from an antibody, for example a human IgG1 or IgG4 hinge region. In an embodiment, the hinge region comprises a sequence having at least 50%, 60%, 70%, 75%, 80%, 85%, 90% or 95% identity with the sequence PEPKSSDKTHTCP (residues 1-13 of SEQ ID NO: 79). In an embodiment, the hinge region comprises or consists of the sequence PEPKSSDKTHTCP.

Modifications can be made to the hinge and Fc region to improve various properties of the scFv-Fc. In one embodiment, one, two, three, four, five or up to ten amino acids of a naturally occurring human Fc region can be modified, in addition to modifications of the hinge region. For example, the Fc region can be modified to increase the serum half-life of the scFv-Fc. The half-life of an IgG depends on its pH-dependent binding to the receptor FcRn. FcRn, which is expressed on the surface of endothelial cells, binds the IgG in a pH-dependent manner and protects it from degradation. Mutations located at the interface between the CH₂ and CH₃ domains, for example, have been shown to increase the binding affinity to FcRn and the half-life of IgG1 in vivo. Such modifications are reviewed in Strohl W R., 2009. Curr Opin Biotechnol. 20(6):685-91; and Vaccaro C. et al., 2005. Nat Biotechnol. 23(10):1283-8, for example.

Other modifications to the hinge and/or Fc fragment can increase or reduce effector functions. The four human IgG isotypes bind the activating Fc-gamma (Fcγ) receptors (FcγRI, FcγRIIa, FcγRIIIa), the inhibitory FcγRIIb receptor, and the first component of complement (Clq) with different affinities, resulting in different effector functions. Binding of IgG to the FcγRs or C1q, for example, depends on residues located in the IgG hinge region and CH₂ domain. Single or multiple amino acid substitutions of these residues can affect effector function by modulating the IgG interaction with FcγRs or C1q. Other substitutions are known to affect effector function. These modifications are reviewed in Strohl 2009 (supra) for example. Amino acid modifications also can be used to improve the pharmacological function of recombinant antibodies or antigen-binding fragments thereof. For example, amino acid modifications can be used to increase complement activation, enhance antibody-dependent cellular cytotoxicity (ADCC) by increasing FcγRIIIA binding or decreasing FcγRIIIB binding, and/or increase serum half-life by increasing FcRn binding. Such amino acid modifications are reviewed in Beck et al. (2010) Nature 10: 345-52, for example.

Representative modifications of the hinge and/or Fc fragment of human IgG1 are summarized in Table 1 (from WO 2016/141244).

TABLE 1

| Iso- | | | | |
|---|---|---|---|---|
| type | Substitution(s) | FcR/C1q binding | Effect | Ref(s) |
| IgG1 | T250Q/M428L | Increased binding | Increased | 1 |
| | | to FcRn | half-life | |
| IgG1 | M252Y/S254T/T256E/ | Increased binding | Increased | 2 |
| | H433K/N434F | to FcRn | half-life | |
| IgG1 | E233P/L234V/L235A | Reduced binding | Reduced | 3, 4 |
| | ΔG236/A327G/A330S/ | to FcyRI | ADCC | |
| | P331S | | and CDC | |
| IgG1 | E333A | Increased binding | Increased | 5, 6 |
| | | to FcyRIIIa | ADCC | |
| | | | and CDC | |
| IgG1 | S239D/A330L/I332E | Increased binding | Increased | 7, 8 |
| | | to FcyRIIIa | ADCC | |
| IgG1 | P257I/Q311I | Increased binding | Same | 9 |
| | | to FcRn | half-life | |
| IgG1 | K326W/E333S | Increased binding | Increased | 10 |
| | | to C1q | CDC | |
| IgG1 | S239D/I332E/G236A | Increased FcyRIIa/ | Increased | 11 |
| | | FcyRIIb ratio | macrophage | |
| | | | phagocytosis | |
| IgG1 | K332A | Reduced binding | Reduced | 5 |
| | | to C1q | CDC | |

1. Hinton et al. (2004) J. Biol. Chem. 279(8):6213-16.
2. Vaccaro et al. (2005) Nature Biotechnol. 23(10): 1283-88.
3. Armour et al. (1999) Eur. J. Immunol. 29(8):2613-24.
4. Shields et al. (2001) J. Biol. Chem. 276(9):6591-604.
5. Idusogie et al. (2000) J. Immunol. 164(8):4178-84.
6. Idusogie et al. (2001) J. Immunol. 166(4):2571-75.
7. Lazar et al. (2006) Proc. Natl Acad. Sci. USA 103(11): 4005-10.
8. Ryan et al. (2007) Mol. Cancer Ther. 6: 3009-18.
9. Datta-Mannan et al. (2007) Drug Metab. Dispos. 35: 86-94.
10. Steurer et al. (1995) J. Immunol. 155(3): 1165-74.
11. Richards et al. (2008) Mol. Cancer Ther. 7(8):2517-27.

Thus, as used herein, the term “Fc fragment” refers to a native Fc fragment from an antibody or a variant thereof (e.g., having at least 60, 65, 70, 75, 80, 85, 90 or 95% sequence identity with a native Fc fragment) maintaining the ability to bind to FcγRs and/or C1q.

### Possible Modifications to the Anti-NTSR1 Antibodies or Antigen-Binding Fragments Thereof

Variations in the anti-NTSR1 antibodies or antigen-binding fragments thereof described herein, can be made, for example, using any of the techniques and guidelines for conservative and non-conservative mutations set forth, for instance, in U.S. Pat. No. 5,364,934. Variations may be a substitution, deletion or insertion of one or more codons encoding the antibody that results in a change in the amino acid sequence as compared with the native sequence antibody. Optionally the variation is by substitution of at least one amino acid with any other amino acid in one or more of the domains of the anti-NTSR1 antibody or antigen-binding fragment thereof. Guidance in determining which amino acid residue may be inserted, substituted or deleted without adversely affecting the desired activity may be found by comparing the sequence of the anti-NTSR1 antibody or antigen-binding fragment thereof with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence. In embodiment, the variant exhibit at least 50%, 55% or 60%, preferably at least 65, 70, 75, 80, 90, 95, 96, 97, 98 or 99% sequence identity with the sequence of the anti-NTSR1 antibody or antigen-binding fragment thereof described herein, and maintain the ability to specifically bind to the conformational epitope from NTSR1 described herein.

“Identity” refers to sequence identity between two polypeptides. Identity can be determined by comparing each position in the aligned sequences. Methods of determining percent identity are known in the art, and several tools and programs are available to align amino acid sequences and determine a percentage of identity including EMBOSS Needle, ClustalW, SIM, DIALIGN, etc. As used herein, a given percentage of identity with respect to a specified subject sequence, or a specified portion thereof, may be defined as the percentage of amino acids in the candidate derivative sequence identical with the amino acids in the subject sequence (or specified portion thereof), after aligning the sequences and introducing gaps, if necessary to achieve the maximum percent sequence identity, as generated by the Smith Waterman algorithm (Smith & Waterman, J. Mol. Biol. 147: 195-7 (1981)) using the BLOSUM substitution matrices (Henikoff & Henikoff, Proc. Natl. Acad. Sci. USA 89:10915-9 (1992)) as similarity measures. A “% identity value” is determined by the number of matching identical amino acids divided by the sequence length for which the percent identity is being reported.

Covalent modifications of anti-NTSR1 antibodies or antigen-binding fragments thereof are included within the scope of this disclosure. Covalent modifications include reacting targeted amino acid residues of an anti-NTSR1 antibody or antigen-binding fragment thereof with an organic derivatizing agent that is capable of reacting with selected side chains or the N- or C-terminal residues of the anti-NTSR1 antibody or antigen-binding fragment thereof. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Other types of covalent modification of the anti-NTSR1 antibody or antigen-binding fragment thereof included within the scope of this disclosure include altering the native glycosylation pattern of the antibody or antigen-binding fragment thereof (Beck et al., Curr. Pharm. Biotechnol. 9: 482-501, 2008; Walsh, Drug Discov. Today 15: 773-780, 2010), and linking the antibody or antigen-binding fragment thereof to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. Glycosylation may also be intentionally altered, for example by inhibiting fucosylation, in order to increase ADCC activity of the resulting antibody or antigen-binding fragment thereof, e.g., scFv-Fc.

In an embodiment, the anti-NTSR1 antibody or antigen-binding fragment thereof is labelled or conjugated with one or more moieties. The anti-NTSR1 antibody or antigen-binding fragment thereof may be labeled with one or more labels such as a biotin label, a fluorescent label, an enzyme label, a coenzyme label, a chemiluminescent label, or a radioactive isotope label. In an embodiment, the anti-NTSR1 antibody or antigen-binding fragment thereof is labelled with a detectable label, for example a fluorescent moiety (fluorophore). Useful detectable labels include fluorescent compounds (e.g., fluorescein isothiocyanate, Texas red, rhodamine, fluorescein, Alexa Fluor® dyes, and the like), radiolabels, enzymes (e.g., horseradish peroxidase, alkaline phosphatase and others commonly used in a protein detection assays), streptavidin/biotin, and colorimetric labels such as colloidal gold, colored glass or plastic beads (e.g., polystyrene, polypropylene, latex, etc.). Chemiluminescent compounds may also be used. Such labelled antibodies or antigen-binding fragments thereof may be useful, for example, for the detection of NTSR1 and/or NTSR1-expressing cells in vivo or in vitro, e.g., by flow cytometry, immunohistochemistry, etc. The anti-NTSR1 antibody or antigen-binding fragment thereof can also be conjugated to detectable or affinity tags that facilitate detection and/or purification of the anti-NTSR1 antibody or antigen-binding fragment thereof. Such tags are well known in the art. Examples of detectable or affinity tags include polyhistidine tags (His-tags), polyarginine tags, polyaspartate tags, polycysteine tags, polyphenylalanine tags, glutathione S-transferase (GST) tags, Maltose binding protein (MBP) tags, calmodulin binding peptide (CBP) tags, Streptavidin/Biotin-based tags, HaloTag®, Profinity eXact® tags, epitope tags (such as FLAG, hemagglutinin (HA), HSV, S/S1, c-myc, KT3, T7, V5, E2, and Glu-Glu epitope tags), reporter tags such as β-galactosidase (β-gal), alkaline phosphatase (AP), chloramphenicol acetyl transferase (CAT), and horseradish peroxidase (HRP) tags (see, e.g., Kimple et al., Curr Protoc Protein Sci. 2013; 73: Unit-9.9).

The anti-NTSR1 antibody or antigen-binding fragment thereof can also be conjugated to one or more therapeutic or active agents (e.g., a drug), and thus may also be used therapeutically to deliver the therapeutic agent(s) (e.g., anti-tumor agent or any other agent useful for the treatment of the disease or condition or for relieving one or more symptoms) into a cell or tissue, such as a tumor. Any method known in the art for conjugating the anti-NTSR1 antibody or antigen-binding fragment thereof to another moiety (e.g., detectable moiety, active agent) may be employed, including those methods described by Hunter et al. (1962) Nature, 144:945; David et al. (1974) Biochemistry, 13: 1014; Pain et al. (1981) J. Immunol. Meth., 40:219; Nygren, J. Histochem. and Cytochem., 30:407 (1982), and Hermanson, Bioconjugate Techniques 1996, Academic Press, Inc., San Diego.

In another aspect, the present disclosure provides an anti-NTSR1 antibody or antigen-binding fragment thereof having one or more of the features described herein, i.e. any combination/sub-combination of the features described herein.

### Nucleic Acids Encoding the Anti-NTSR1 Antibody or Antigen-Binding Fragment Thereof, Cells and Methods of Making

A further aspect of the present disclosure provides nucleic acids encoding the anti-NTSR1 antibody or antigen-binding fragment described herein. The isolated nucleic acid may be a synthetic DNA, a non-naturally occurring mRNA, or a cDNA, for example. Examples include the nucleic acids encoding the V_{H} and V_{L} domains and/or the nucleic acids encoding the scFv or scFv-Fc described herein. The nucleic acid may be inserted within a plasmid, vector, or transcription or expression cassette. The nucleic acids encoding the anti-NTSR1 antibody or antigen-binding fragment described herein may be made and the expressed antibodies or antigen-binding fragments described may be tested using conventional techniques well known in the art.

In another aspect, the present invention provides a cell, for example a recombinant host cell, expressing the anti-NTSR1 antibody or antigen-binding fragment described herein. Methods of preparing antibodies or antigen-binding fragments comprise expressing the encoding nucleic acid(s) in a host cell under conditions to produce the antibodies or antigen-binding fragments, and recovering the antibodies or antigen-binding fragments. The process of recovering the antibodies or antigen-binding fragments may comprise isolation and/or purification of the antibodies or antigen-binding fragments. The method of production may comprise formulating the antibodies or antigen-binding fragments into a composition including at least one additional component, such as a pharmaceutically acceptable excipient.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred enkaryotic cells include protist, fungal, plant and animal cells. Most preferably host cells include but are not limited to the prokaryotic cell line E. Coli; mammalian cell lines CHO, HEK 293 and COS; the insect cell line Sf9; the fungal cell Saccharomyces cerevisiae, plant cells, or algae cells.

In another embodiment, the host cell is an immune cell. The anti-NTSR1 antibody or antigen-binding fragment described herein may be used as a chimeric antigen receptor (CAR) to produce CAR T cells, CAR NK cells, etc. CAR combines a ligand-binding domain (e.g. antibody or antibody fragment) that provides specificity for a desired antigen (e.g., NTSR1) with an activating intracellular domain (or signal transducing domain) portion, such as a T cell or NK cell activating domain, providing a primary activation signal. Antigen-binding fragments of antibodies, and more particularly scFv, capable of binding to molecules expressed by tumor cells are commonly used as CAR. Thus, in another aspect, the present disclosure provides a host cell, preferably an immune cell such as a T cell or NK cell, expressing the scFv described herein.

The CAR of the present disclosure may also comprise a transmembrane domain which spans the membrane. The transmembrane domain may be derived from a natural polypeptide, or may be artificially designed. The transmembrane domain derived from a natural polypeptide can be obtained from any membrane-binding or transmembrane protein. For example, a transmembrane domain of a T cell receptor a or β chain, CD28, CD3-epsilon, CD45, CD4, CD5, CD8, CD9, CD16, CD22, CD33, CD37, CD64, CD80, CD86, CD134, CD137, ICOS, CD 154, or a GITR can be used. The artificially designed transmembrane domain is a polypeptide mainly comprising hydrophobic residues such as leucine and valine. It is preferable that a triplet of phenylalanine, tryptophan and valine is found at each end of the synthetic transmembrane domain. In preferred embodiments, the transmembrane domain is derived from CD28 or CD8, which give good receptor stability.

Preferred examples of signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic signaling sequence, those that initiate antigen-dependent primary activation, and those that act in an antigen-independent manner to provide a secondary or co-stimulatory signal. Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non-limiting examples those derived from TCRzeta, FcRgamma, FcRbeta, FcRepsilon, CD3gamma, CD3delta, CD3epsilon, CD5, CD22, CD79a, CD79b and CD66d.

The CAR of the present disclosure may also comprise one or more co-stimulatory domains such as human CD28, 4-1BB (CD137), ICOS-1, CD27, OX40 (CD137), DAP10, and GITR (AITR). In embodiment, the CAR is a third generation and comprises two co-stimulating domains such as CD28 and 4-1BB.

The CAR of the present disclosure may also comprise a signal peptide N-terminal to the anti-NTSR1 antibody or antigen-binding fragment described herein (e.g., scFv) so that when the CAR is expressed inside a cell, such as a T-cell, the nascent protein is directed to the endoplasmic reticulum and subsequently to the cell surface, where it is expressed. The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases. As an example, the signal peptide may derive from human CD8 or GM-CSF, or a variant thereof having 1 or 2 amino acid mutations provided that the signal peptide still functions to cause cell surface expression of the CAR.

The CAR of the present disclosure may comprise a spacer sequence as a hinge to connect the anti-NTSR1 antibody or antigen-binding fragment described herein (e.g., scFv) with the transmembrane domain and spatially separate antigen binding domain from the endodomain. A flexible spacer allows to the binding domain to orient in different directions to enable its binding to the desired antigen (e.g., NTSR1). The spacer sequence may, for example, comprise an IgG1 Fc region, an IgG1 hinge or a CD8 stalk, or a combination thereof.

Suitable vectors comprising nucleic acid(s) encoding the anti-NTSR1 antibody or antigen-binding fragment described herein can be chosen or constructed, containing appropriate regulatory sequences, including promoter sequences, terminator sequences, polyadenylation sequences, enhancer sequences, marker genes and other sequences as appropriate. Vectors may be plasmids, phage, phagemids, adenoviral, AAV, lentiviral, for example. Techniques and protocols for manipulation of nucleic acid, for example in preparation of nucleic acid constructs, mutagenesis, sequencing, introduction of DNA into cells, and gene expression, are well known in the art.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Introducing such nucleic acids into a host cell can be accomplished using techniques well known in the art. For eukaryotic cells, suitable techniques may include calcium phosphate transfection, DEAE-Dextran, electroporation, liposome-mediated transfection, and transduction using retroviruses or other viruses, for example. For bacterial cells, suitable techniques may include calcium chloride transformation, electroporation, and transfection using bacteriophage. The introduction may be followed by causing or allowing expression from the nucleic acid, e.g. by culturing host cells under conditions for expression of the gene. In one embodiment, the nucleic acid of the invention is integrated into the genome, e.g., chromosome, of the host cell. Integration may be promoted by inclusion of sequences which promote recombination with the genome, in accordance with standard techniques.

Systems for cloning and expression of a polypeptide in a variety of different host cells are well known. Suitable host cells include bacteria, mammalian cells, plant cells, insect cells, fungi, yeast and transgenic plants and animals. Mammalian cell lines available in the art for expression of a heterologous polypeptide include Chinese hamster ovary (CHO) cells, HeLa cells, baby hamster kidney cells, mouse melanoma cells, rat myeloma cells, human embryonic kidney cells, e.g., HEK293 cells, human embryonic retina cells, and many others. The expression of antibodies and antibody fragments in prokaryotic cells, such as E. coli, is well established in the art. For a review, see for example, Pliickthun Bio/Technology 9: 545-551 (1991). Expression in cultured eukaryotic cells is also available to those skilled in the art, as reviewed in Andersen et al. (2002) Curr. Opin. Biotechnol. 13: 117-23, for example.

Compositions Comprising the Anti-NTSR1 Antibodies or Antigen-Binding Fragments Thereof.

In another aspect, the present disclosure provides a composition comprising the anti-NTSR1 antibody or an antigen-binding fragment thereof defined herein. In an embodiment, the composition further comprises the above-mentioned anti-NTSR1 antibody or an antigen-binding fragment thereof and a carrier or excipient, in a further embodiment a pharmaceutically acceptable carrier or excipient. Such compositions may be prepared in a manner well known in the pharmaceutical art by mixing the antibody or an antigen-binding fragment thereof having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration of the antibody or an antigen-binding fragment thereof by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the carrier/excipient is adapted for administration of the antibody or an antigen-binding fragment thereof by the intravenous or subcutaneous route. In an embodiment, the carriers/excipients are adapted for administration of the antibody or an antigen-binding fragment thereof by the intravenous route. In another embodiment, the carriers/excipients are adapted for administration of the antibody or an antigen-binding fragment thereof by the subcutaneous route.

An “excipient” as used herein has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipient include water, saline, phosphate buffered saline, dextrose, glycerol, ethanol, and the like, as well as combinations thereof. In many cases, it will be preferable to include isotonic agents, for example, sugars, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness.

The composition may also comprise one or more additional active agents for the treatment the targeted disease/condition or for the management of symptom(s) of the targeted disease/condition (e.g., pain killers, anti-nausea agents, etc.), as described in more detail below.

### Uses of the Anti-NTSR1 Antibodies and Antigen-Binding Fragments Thereof or Compositions Comprising Same

In another aspect, the present disclosure provides a method for inhibiting NTSR1 activity in a cell, the method comprising contacting the cell with an effective amount of the anti-NTSR1 antibody or antigen-binding fragment thereof or composition comprising same described herein. The present disclosure also provides the use of the anti-NTSR1 antibody or antigen-binding fragment thereof or composition comprising same described herein for inhibiting NTSR1 activity in a cell, or for the manufacture of a medicament for inhibiting NTSR1 activity in a cell. In an embodiment, the NTSR1 activity is mediated by binding of neurotensin (NT) to NTSR1. In an embodiment, the NTSR1 activity comprises NT-mediated G_{αq} activation, i.e. activation of the G_{αq} pathway induced by the binding of NT to NTSR1.

In another aspect, the present disclosure provides a method for inhibiting the binding of neurotensin (NT) to NTSR1 on a cell, the method comprising contacting the cell (NTSR1-expressing cell) with an effective amount of the anti-NTSR1 antibody or antigen-binding fragment thereof or composition comprising same described herein. The present disclosure also provides the use of the anti-NTSR1 antibody or antigen-binding fragment thereof or composition comprising same described herein for inhibiting the binding of NT to NTSR1 on a cell, or for the manufacture of a medicament for inhibiting the binding of NT to NTSR1 on a cell. In an embodiment, the cell is a tumor or cancer cell. In an embodiment, the cell is a pancreatic, prostate, lung, breast, liver or colon cell. In another embodiment, the cell is a pancreatic or liver cell.

In another aspect, the present disclosure provides a method for treating a disease or condition associated with NTSR1 activity (e.g., dysregulated or abnormal NTSR1 activity) in a subject, the method comprising administering to the subject an effective amount of the anti-NTSR1 antibody or antigen-binding fragment thereof or composition comprising same described herein. The present disclosure also provides the use of the anti-NTSR1 antibody or antigen-binding fragment thereof or composition comprising same described herein for treating a disease or condition associated with NTSR1 activity in a subject, or for the manufacture of a medicament for treating a disease or condition associated with NTSR1 activity in a subject.

NTSR1 belongs to the large superfamily of GPCRs and has been shown to mediate the multiple functions of NT, such as hypotension, hyperglycemia, hypothermia, antinociception, and regulation of intestinal motility and secretion.

NTSR1 expression/overexpression has been shown to be associated with inflammatory bowel diseases (see, e.g., Gui et al., World J. Gastroenterol. 2013 Jul. 28; 19(28): 4504-4510. Accordingly, in an embodiment, the disease or condition associated with NTSR1 activity is an inflammatory bowel disease (IBD), e.g., Crohn's disease or ulcerative colitis (UC). In an embodiment, the treatment reduces intestinal mucosal inflammation and/or acute colonic inflammation. In an embodiment, the treatment prevents or reduces the risk of developing colitis-associated neoplasia.

There is also evidence that the NT/NTSR1 system is involved in certain metabolic disorders such as nonalcoholic fatty liver disease (NAFLD) and nonalcoholic steatohepatitis (NASH), as higher plasma pro-NT levels were detected in subjects with NAFLD, which also correlated with the severity of the disease (Barchetta et al., The Journal of Clinical Endocrinology & Metabolism, 103(6): 2253-2260). Higher plasma pro-NT levels have also been associated with features of insulin resistance and doubled the risk of developing obesity later in life in non-obese individuals (Li J et al., Nature. 2016; 533(7603):411-415). Systemically administered NT is known to have a hyperglycemic and hyperglucagonemic effect. These effects have been found to be blocked by histamine H-1 and H-2 receptor (Nagai K, Frohman L A. Diabetes. 1978 27(5):577-82), suggesting that histamine release is involved in the signaling pathway. Changes in somatostatin were also reported. NT-deficient mice were shown to be more resistant to high-fat-diet induced obesity, insulin resistance and accumulation of lipid in liver tissue (Jing L., et al. Nature 533, 411-415).

Thus, in an embodiment, the disease or condition associated with NTSR1 activity is a cardiovascular or metabolic disease, such as obesity, insulin resistance, glucose resistance, type 2 diabetes, NAFLD or NASH. In an embodiment, the disease or condition associated with NTSR1 activity is NAFLD or NASH.

In addition to the physiological actions of NTSR1 engagement by NT in the central nervous system (CNS) and gastrointestinal tract, there is increasing evidence that NT-mediated NTSR1 stimulation plays an important role in carcinogenesis. NT oncogenic action has been described in numerous types of cancer cells and tumors with abnormal expression of NTSR1 (e.g., pancreatic, prostate, lung, breast, and colon cancer), with effects in each step of cancer progression from tumor growth, with proliferative and survival effects, to metastatic spread, with anchorage independent growth, and pro-migratory and pro-invasive effects (see, e.g., Wu et al., Front Endocrinol (Lausanne). 2012; 3: 184; Moody et al., Life Sci. 2014; 100(1): 25-34; Wu et al., Front Endocrinol 2013; 3: 1-9; Alfano et al., Clin Cancer Res. 2010; 16:4401-4410; Dupouy et al., PLoS ONE. 2009; 4:e4223; Shimizu et al., Int J Cancer. 2008; 123:1816-1823. Moody et al., Peptides. 2001; 22:109-115; Maoret et al., Int J Cancer. 1999; 80:448-454). All these effects are associated with the abnormal or dysregulated expression of NTSR1 during the early stages of cell transformation, and are the result from the activation of several kinases and effectors such as PKC, MAPK, FAK, RHO-GTPase, RAS and Scr. NTSR1 expression has been shown to be an independent marker of poor prognosis in several cancers including breast, lung, and head and neck squamous cell carcinomas.

In another aspect, the present disclosure provides a method for treating cancer (NTSR1-positive cancer) in a subject, the method comprising administering to the subject an effective amount of the anti-NTSR1 antibody or antigen-binding fragment thereof or composition comprising same described herein. The present disclosure also provides the use of the anti-NTSR1 antibody or antigen-binding fragment thereof or composition comprising same described herein for treating cancer (NTSR1-positive cancer) in a subject, or for the manufacture of a medicament for treating cancer (NTSR1-positive cancer) in a subject.

In an embodiment, the above-noted cell is a tumor or cancer cell expressing NTSR1. In an embodiment, the tumor or cancer cell also expresses/secretes NT.

In an embodiment, the NTSR1-expressing tumor/cancer is heart sarcoma, lung cancer, small cell lung cancer (SCLC), non-small cell lung cancer (NSCLC), bronchogenic carcinoma (squamous cell, undifferentiated small cell, undifferentiated large cell, adenocarcinoma), alveolar (bronchiolar) carcinoma, bronchial adenoma, sarcoma (e.g., Ewing's sarcoma, Karposi's sarcoma), lymphoma, chondromatous hamartoma, mesothelioma; cancer of the gastrointestinal system, for example, esophagus (squamous cell carcinoma, adenocarcinoma, leiomyosarcoma, lymphoma), stomach (carcinoma, lymphoma, leiomyosarcoma), gastric, pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); cancer of the genitourinary tract, for example, kidney cancer (adenocarcinoma, Wilm's tumor [nephroblastoma], lymphoma, leukemia), bladder and/or urethra cancer (squamous cell carcinoma, transitional cell carcinoma, adenocarcinoma), prostate cancer (adenocarcinoma, sarcoma), testis cancer (seminoma, teratoma, embryonal carcinoma, teratocarcinoma, choriocarcinoma, sarcoma, interstitial cell carcinoma, fibroma, fibroadenoma, adenomatoid tumors, lipoma); liver cancer, for example, hepatoma (hepatocellular carcinoma, HCC), cholangiocarcinoma, hepatoblastoma, angiosarcoma, hepatocellular adenoma, hemangioma, pancreatic endocrine tumors (such as pheochromocytoma, insulinoma, vasoactive intestinal peptide tumor, islet cell tumor and glucagonoma); bone cancer, for example, osteogenic sarcoma (osteosarcoma), fibrosarcoma, malignant fibrous histiocytoma, chondrosarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; cancer of the nervous system, for example, neoplasms of the central nervous system (CNS), primary CNS lymphoma, skull cancer (osteoma, hemangioma, granuloma, xanthoma, osteitis deformans), meninges (meningioma, meningiosarcoma, gliomatosis), brain cancer (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); cancer of the reproductive system, for example, gynecological cancer, uterine cancer (endometrial carcinoma), cervical cancer (cervical carcinoma, pre-tumor cervical dysplasia), ovarian cancer (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulvar cancer (squamous cell carcinoma, intraepithelial carcinoma, adenocarcinoma, fibrosarcoma, melanoma), vaginal cancer (clear cell carcinoma, squamous cell carcinoma, botryoid sarcoma (embryonal rhabdomyosarcoma), fallopian tube cancer (carcinoma); placenta cancer, penile cancer, prostate cancer, testicular cancer; cancer of the hematologic system, for example, blood cancer (acute myeloid leukemia (AML), chronic myeloid leukemia (CML), acute lymphoblastic leukemia (ALL), chronic lymphocytic leukemia (CLL), myeloproliferative diseases, multiple myeloma, myelodysplastic syndrome), Hodgkin's disease, non-Hodgkin's lymphoma [malignant lymphoma]; cancer of the oral cavity, for example, lip cancer, tongue cancer, gum cancer, palate cancer, oropharynx cancer, nasopharynx cancer, sinus cancer; skin cancer, for example, malignant melanoma, cutaneous melanoma, basal cell carcinoma, squamous cell carcinoma, Karposi's sarcoma, moles dysplastic nevi, lipoma, angioma, dermatofibroma, and keloids; adrenal gland cancer: neuroblastoma; and cancers of other tissues including connective and soft tissue, retroperitoneum and peritoneum, eye cancer, intraocular melanoma, and adnexa, breast cancer (e.g., ductal breast cancer), head or/and neck cancer (head and neck squamous cell carcinoma), anal cancer, thyroid cancer, parathyroid cancer; secondary and unspecified malignant neoplasm of lymph nodes, secondary malignant neoplasm of respiratory and digestive systems and secondary malignant neoplasm of other sites.

In a further embodiment, the NTSR1-expressing tumor/cancer is a pancreatic cancer (e.g., pancreatic ductal adenocarcinoma), prostate cancer (e.g., prostate adenocarcinoma), lung cancer (e.g., small cell lung carcinoma (SCLC), non-small cell lung adenocarcinoma (NSCLC), pleural mesothelioma), breast cancer, thyroid cancer (medullary thyroid carcinoma), liver cancer (e.g., hepatocellular carcinoma, HCC), brain cancer (e.g., glioma, glioblastoma), uterine cancer (e.g., endometrial adenocarcinoma), bladder cancer, ovarian cancer, skin cancer (e.g., skin melanoma), gastric cancer or colorectal cancer (e.g., colon adenocarcinoma). In an embodiment, the tumor or cancer is a primary tumor or cancer. In another embodiment, the tumor or cancer is a metastatic or secondary tumor or cancer. In another embodiment, the tumor or cancer is a refractory tumor or cancer. In an embodiment, the treatment is for reducing cancer's aggressiveness and/or metastatic potential.

In an embodiment, the cancer or tumor expresses or overexpresses an epidermal growth factor receptor (e.g., EGFR/HER1, HER2, HER3, and/or HER4), for example a constitutively activated form of an epidermal growth factor receptor.

In an embodiment, the anti-NTSR1 antibody or antigen-binding fragment thereof interferes with the binding of NT to NTSR1 on the target cell and/or inhibits the NT-mediated activation of one or more of the NTSR1-associated signaling pathways in the target cell.

In another embodiment, the anti-NTSR1 antibody or antigen-binding fragment thereof induces the killing of the NTSR1-expressing cell (e.g., NTSR1-expressing tumor/cancer cell) through complement-dependent cytotoxicity (CDC) or antibody-dependent cell-mediated (or cellular) cytotoxicity (ADCC). In a further embodiment, the anti-NTSR1 antibody or antigen-binding fragment thereof interferes with the binding of NT to NTSR1 and/or inhibits the NT-mediated activation of one or more of the NTSR1-associated signaling pathways in the target cell, and induces the killing of the NTSR1-expressing cell through CDC or ADCC. In an embodiment, the anti-NTSR1 antibody or antigen-binding fragment thereof comprises a domain that is recognized by a component of the classical complement pathway and/or by cytotoxic immune cells (e.g., NK cells macrophages, monocytes or eosinophils), for example a domain that is recognized by the initiating component Clq of the classical complement pathway and/or by a Fc gamma receptor present on cytotoxic immune cells (e.g., a fragment crystallizable (Fc) portion), and induces the killing of the NTSR1-expressing cell (e.g., NTSR1-expressing tumor/cancer cell) through CDC and/or ADCC.

As used herein, the term “effective amount” refers to a quantity of anti-NTSR1 antibody or antigen-binding fragment thereof sufficient to achieve a desired biological, therapeutic and/or prophylactic effect, e.g., an amount which results in inhibition/reduction of NTSR1 activity in a cell, or in the prevention of, or a decrease in, the symptoms associated with cancer. The amount of the anti-NTSR1 antibody or antigen-binding fragment thereof used or administered to the subject will depend, for example, on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The anti-NTSR1 antibody or antigen-binding fragment thereof may also be administered in combination with one or more additional therapeutic compounds. In the methods described herein, the anti-NTSR1 antibody or antigen-binding fragment thereof may be administered to a subject having one or more signs or symptoms of cancer. For example, a “therapeutically effective amount” of the anti-NTSR1 antibody or antigen-binding fragment thereof is meant levels in which the physiological effects of cancer are, at a minimum, ameliorated.

The anti-NTSR1 antibody or antigen-binding fragment thereof or composition comprising same described herein may be used in combination with one or more additional active agents or therapies (radiotherapy, surgery, vaccines, etc.) for the treatment the targeted disease/condition or for the management of one or more symptoms of the targeted disease/condition (e.g., pain killers, anti-nausea agents, etc.). In an embodiment, the anti-NTSR1 antibody or antigen-binding fragment thereof described herein is used in combination with one or more chemotherapeutic agents, immunotherapies, checkpoint inhibitors, cell-based therapies, etc. Examples of chemotherapeutic agents suitable for use in combination with the anti-NTSR1 antibody or antigen-binding fragment thereof described herein include, but are not limited to, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, retinoids (such as all-trans retinoic acids ora derivatives thereof); geldanamycin ora derivative thereof (such as 17-AAG), and other cancer therapeutic agents recognized in the art. In some embodiments, chemotherapeutic agents for use in combination with the anti-NTSR1 antibody or antigen-binding fragment thereof described herein comprise one or more of adriamycin, colchicine, cyclophosphamide, actinomycin, bleomycin, duanorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., taxol, paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Oxaliplatin, Herceptin®, vinorelbine, Doxil®, capecitabine, Alimta®, Avastin®, Velcade®, Tarceva®, Neulasta®, lapatinib, sorafenib, erlotinib, erbitux, derivatives thereof, and the like. In an embodiment, the anti-NTSR1 antibody or antigen-binding fragment thereof or composition comprising same described herein is used in combination with an EGFR or tyrosine kinase targeting agent, for example an EGFR inhibitor (RTK inhibitor). The anti-NTSR1 antibody or antigen-binding fragment thereof or composition comprising same described herein may also be used in combination with one or more additional therapeutic antibodies or antibody fragments, e.g., therapeutic antibodies or antibody fragments used for the treatment of tumors.

The combination of active agents and/or compositions comprising same may be administered or co-administered (e.g., consecutively, simultaneously, at different times) in any conventional dosage form. Co-administration in the context of the present invention refers to the administration of more than one therapeutic in the course of a coordinated treatment to achieve an improved clinical outcome. Such co-administration may also be coextensive, that is, occurring during overlapping periods of time. For example, a first agent (e.g., the anti-NTSR1 antibody or antigen-binding fragment thereof described herein) may be administered to a patient before, concomitantly, before and after, or after a second active agent (e.g., a chemotherapeutic agent) is administered. The agents may in an embodiment be combined/formulated in a single composition and thus administered at the same time.

In an embodiment, the anti-NTSR1 antibody or antigen-binding fragment thereof or composition comprising same described herein is used as a targeting agent or delivery vehicle to deliver an agent of interest (e.g., a drug such as a chemotherapeutic agent, a radionuclide, an imaging agent) to an NTSR1-expressing cell or tissue. In an embodiment, the agent of interest is conjugated to the anti-NTSR1 antibody or antigen-binding fragment thereof.

As used herein, the term “subject” is taken to mean warm blooded animals such as mammals, for example, cats, dogs, mice, guinea pigs, horses, bovine cows, sheep and humans. In an embodiment, the subject is a mammal, and more particularly a human.

In another aspect, the present disclosure relates to a method for detecting NTSR1 in a sample or on a cell comprising contacting the cell with the anti-NTSR1 antibody or antigen-binding fragment thereof described herein. In an embodiment, the method further comprises detecting or quantifying the antibody-NTSR1 complexes. Such method may be useful to detect, quantify and/or purify NTSR1-expressing cells in a sample, for example for diagnosing a disease or condition associated with dysregulated NTSR1 expression/activity, such as the diseases or conditions described above (e.g., an NTSR1-expressing cancer). In an embodiment, the anti-NTSR1 antibody or antigen-binding fragment thereof described herein is conjugated to a detectable label (e.g., an imaging agent), as described above.

### Cyclic Peptides

As noted above, the present inventors have engineered a cyclic peptide mimicking the conformation of the second extracellular loop of NTSR1, which was used to immunize chicken and raise antibodies specifically recognizing NTSR1.

Thus, in another aspect, the present disclosure provides a cyclic peptide comprising a domain of the following sequence:

X¹—X²—X³—N—R—S-A-D-G¹-X⁴—H—X⁵-G²-G³
wherein
“—” are bonds, preferably peptide (amide) bonds;
X¹ is an amino acid or amino acid analog, preferably cysteine (C), or is absent;
X² is an amino acid or amino acid analog, preferably lysine or a lysine analog, or is absent;
X³ is an amino acid or amino acid analog, preferably an amino acid comprising a lateral chain comprising an amine or an analog thereof, more preferably lysine (K), or is absent;
X⁴ and X⁵ are any amino acid;
one of X³ or N is attached via a bond, preferably a peptide bond, to G³, thereby forming a cyclic peptide;
or a salt thereof.

In an embodiment, X¹ and X² are absent. In another embodiment, X¹ and X² are present. In a further embodiment, X¹ is a cysteine (C) residue. In another further embodiment, X² is a lysine (K) residue.

In an embodiment, X³ is absent. In another embodiment, X³ is present. If present, X³ is preferably lysine or ornithine, preferably lysine. In a further embodiment, the amine group of X³ (e.g., lysine or ornithine) forms a peptide bond with the carboxy-terminal end of G³.

In an embodiment, X⁵ is Q or T. In an embodiment, X⁵ is Q. In another embodiment, X⁵ is T.

In an embodiment, X⁶ is A or P. In an embodiment, X⁶ is A. In another embodiment, X⁶ is P.

In another aspect, the present invention provides a cyclic peptide of formula I:

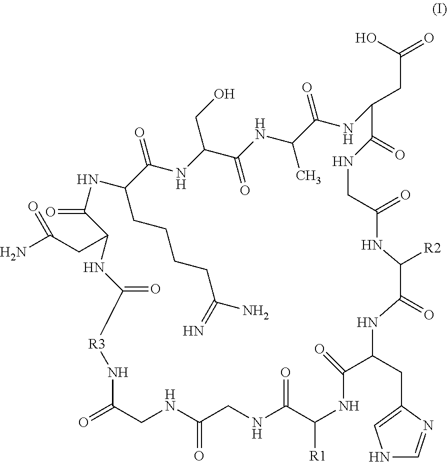

wherein
R1 and R2 are independently the side chain of any amino acid or amino acid analog;
R3 is a substituted or unsubstituted C₁-C₈ alkyl;

In an embodiment, R1 is the side chain of alanine (A) or proline (P). In an embodiment, R1 is the side chain of A. In another embodiment, R1 is the side chain of P.

In an embodiment, R2 is the side chain of glutamine (Q) or threonine (T). In an embodiment, R2 is the side chain of Q. In another embodiment, R2 is the side chain of T.

In an embodiment, the cyclic peptide is of formula Ia:

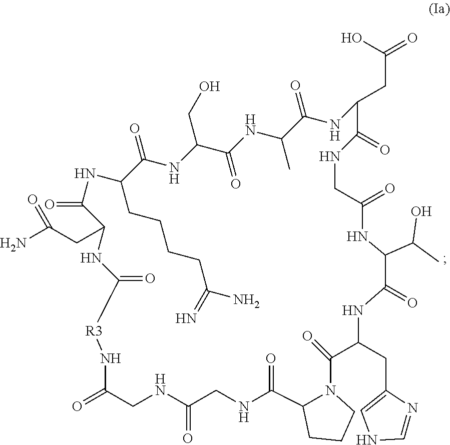

wherein R3 is as defined above.

In an embodiment, R3 is a substituted or unsubstituted C₁-C₆ alkyl, preferably a substituted or unsubstituted C₁-C₄ alkyl.

In an embodiment, R3 is substituted with a R4 substituent, wherein R4 is a linker for conjugating the cyclic peptide to a moiety/molecule.

Thus, in an embodiment, the present disclosure provides a conjugate or fusion molecule of formula II:

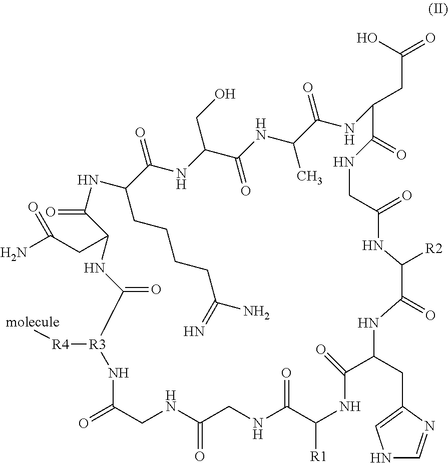

In an embodiment, R4 comprises a thiol (SH) group.

In a further embodiment, R4 is (CH₂)ₘ—NH(CO)—C(NR5R6)-(CH₂)ₙ—SH or NH(CO)—(CH₂)ₘ—NH(CO)—C(NR5R6)-(CH₂)ₙ—SH, wherein m and n are independently an integer from 1 to 6, R5 and R6 are independently H or C₁-C₆ alkyl. In an embodiment, m is an integer from 3 to 5, preferably 4. In an embodiment, n is an integer from 1 to 3, preferably 1. In an embodiment, at least one of R5 and R6 is H, preferably R5 and R6 are H.

In an embodiment, R3 is

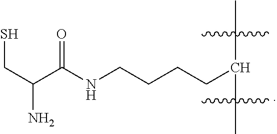 [SMILES: *C(*)CCCCNC(=O)C(N)CS]

In another embodiment, R3 is

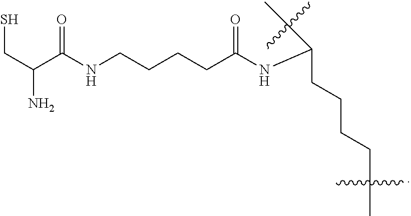 [SMILES: *CCCCC(*)NC(=O)CCCCNC(=O)C(N)CS]

The term “salt(s)”, as employed herein, denotes acidic salts formed with inorganic and/or organic acids, as well as basic salts formed with inorganic and/or organic bases. Salts for use in pharmaceutical compositions will be pharmaceutically acceptable salts, but other salts may be useful in the production of the cyclic peptide described herein. As used herein the term “pharmaceutically acceptable salt” refers to salts of peptides that retain the biological activity of the parent peptide, and which are not biologically or otherwise undesirable.

The term “amino acid” as used herein includes both L- and D-isomers of the naturally occurring amino acids as well as other amino acids (e.g., naturally-occurring amino acids, non-naturally-occurring amino acids, amino acids which are not encoded by nucleic acid sequences, etc.) used in peptide chemistry to prepare synthetic analogs of peptides. Examples of naturally-occurring amino acids are glycine, alanine, valine, leucine, isoleucine, serine, threonine, etc.

Other amino acids include for example non-genetically encoded forms of amino acids, as well as a conservative substitution of an L-amino acid. Naturally-occurring non-genetically encoded amino acids include, for example, beta-alanine, 3-amino-propionic acid, 2,3-diamino propionic acid, alpha-aminoisobutyric acid (Aib), 4-amino-butyric acid, N-methylglycine (sarcosine), hydroxyproline, ornithine (e.g., L-ornithine), citrulline, t-butylalanine, t-butylglycine, N-methylisoleucine, phenylglycine, cyclohexylalanine, norleucine (Nle), norvaline, 2-napthylalanine, pyridylalanine, 3-benzothienyl alanine, 4-chlorophenylalanine, 2-fluorophenylalanine, 3-fluorophenylalanine, 4-fluorophenylalanine, penicillamine, 1,2,3,4-tetrahydro-isoquinoline-3-carboxylix acid, beta-2-thienylalanine, methionine sulfoxide, L-homoarginine (Hoarg), N-acetyl lysine, 2-amino butyric acid, 2-amino butyric acid, 2,4,-diaminobutyric acid (D- or L-), p-aminophenylalanine, N-methylvaline, homocysteine, homoserine (HoSer), cysteic acid, epsilon-amino hexanoic acid, delta-amino valeric acid, or 2,3-diaminobutyric acid (D- or L-), etc. These amino acids are well known in the art of biochemistry/peptide chemistry.

The above-noted cyclic peptide may comprise all L-amino acids, all D-amino acids or a mixture of L- and D-amino acids. As such, the single-letter code for designing amino acids in the above-noted formula encompass both the L- and D-isomers of the recited amino acids (for those having a chiral center). For example, the letter “N” refers to L-asparagine and D-asparagine.

To form the cyclic peptide, one of X¹, X², X³, X⁴ or R is attached via a bond to one of G³, X⁷ or X⁸. The bond/connection may be between the amino-terminal and carboxy-terminal ends of the peptide, between the amino-terminal end and a side chain of an amino acid, between the carboxy-terminal end and a side chain of an amino acid, or between two side chains of amino acids. The two amino acids may be bonded or connected through any suitable bond, including a normal peptide bond (i.e. between the alpha carboxyl of one residue to the alpha amine of another non-contiguous residue), a non-alpha amide bond, such as a bond between the side chain of one residue to the alpha carboxyl group of another residue, or any other chemically stable bonds such as lactone, ether, thioether, or disulfide.

In embodiments, the above-mentioned cyclic peptide may comprise, further to the domain defined above, one more amino acids (naturally occurring or synthetic) covalently linked to the amino- and/or carboxy-termini of said domain. In an embodiment, the above-mentioned cyclic peptide comprises up to 5 additional amino acids at the N- and/or C-termini to the domain defined above. In further embodiments, the above-mentioned GRF analog comprises up to 5, 4, 3, 2, or 1 additional amino acids at the N- and/or C-termini of the domain defined above. In an embodiment, the above-mentioned domain or cyclic peptide comprises about 50 residues or less, preferably 40, 30, 20, 15, 14, or 13 amino acids or less. In an embodiment, the above-mentioned domain or cyclic peptide comprises about 9 or 10 to about 15 residues, preferably about 11 to about 15 residues, for example 12, 13 or 14 amino acids. In an embodiment, the above-mentioned cyclic peptide consists of the domain defined above.

In an embodiment, the native amino-terminal and/or carboxy-terminal end of the peptide may be modified using amino-terminal and/or carboxy-terminal modifying groups.

The term “amino-terminal modifying group” refers to a moiety commonly used in the art of peptide chemistry to replace or modify the native NH₂ terminal group of the cyclic peptide, for example to increase its stability and/or susceptibility to protease digestion. The amino-terminal modifying group may be a straight chained or branched alkyl group of one to eight carbons, or an acyl group (R^{A}—CO—), wherein R^{A} is a hydrophobic moiety (e.g., alkyl, such as methyl, ethyl, propyl, butanyl, iso-propyl, or iso-butanyl), or an aroyl group (Ar—CO—), wherein Ar is an aryl group. The acyl group may be a C₁-C₁₆ or C₃-C₁₆ acyl group (linear or branched, saturated or unsaturated), such as a saturated C₁-C₆ acyl group (linear or branched) or an unsaturated C₃-C₆ acyl group (linear or branched), for example an acetyl group (CH₃—CO—, Ac). In an embodiment, when the amino terminal end of the peptide does not form the bond that makes the peptide cyclic, the cyclic peptide has a native NH₂ terminal group.

The term “carboxy-terminal modifying group” refers to a moiety commonly used in the art of peptide chemistry to replace or modify the native CO₂H terminal group of the peptide, for example to increase its stability and/or susceptibility to protease digestion. The carboxy-terminal modifying group may be:
a hydroxylamine group (NHOH) attached to the carboxyl group (—C(═O)—NHOH),
an amine attached to the carboxyl group (—C(═O)—NR^{B}R^{C}, the amine being a primary, secondary or tertiary amine, and preferably the amine is an aliphatic amine preferably of one to ten carbons, such as methyl amine, iso-butylamine, iso-valerylamine or cyclohexylamine, an aromatic amine or an arylalkyl amine, such as aniline, napthylamine, benzylamine, cinnamylamine, or phenylethylamine, a preferred amine being —NH₂,
a nitrile group (CEN), or
a hydroxyalkyl (i.e. an alcohol), preferably CH₂OH.

In an embodiment, when the carboxyl terminal end of the peptide does not form the bond that makes the peptide cyclic, the cyclic peptide has a native CO₂H terminal group.

The cyclic peptide may also be a peptidomimetic. A peptidomimetic is typically characterised by retaining the polarity, three-dimensional size and functionality (bioactivity) of its peptide equivalent, but wherein one or more of the peptide bonds/linkages have been replaced, often by more stable linkages. Generally, the bond which replaces the amide bond (amide bond surrogate) conserves many or all of the properties of the amide bond, e.g. conformation, steric bulk, electrostatic character, potential for hydrogen bonding, etc. Typical peptide bond replacements include esters, polyamines and derivatives thereof as well as substituted alkanes and alkenes, such as aminomethyl and ketomethylene. For example, the above-mentioned domain or cyclic may have one or more peptide linkages replaced by linkages such as —CH₂NH—, —CH₂S—, —CH₂—CH₂—, —CH═CH— (cis or trans), —CH₂SO—, —CH(OH)CH₂—, or —COCH₂—. Such peptidomimetics may have greater chemical stability, enhanced biological/pharmacological properties (e.g., half-life, absorption, potency, efficiency, etc.) and/or reduced antigenicity relative its peptide equivalent. In an embodiment, the cyclic peptide only comprises native peptide bonds except for the bond between one of X¹, X², X³, X⁴ or R and one of G³, X⁷ or X⁸ that forms the cycle, i.e. the bonds represented by “—” in the formula above are all peptide bonds.

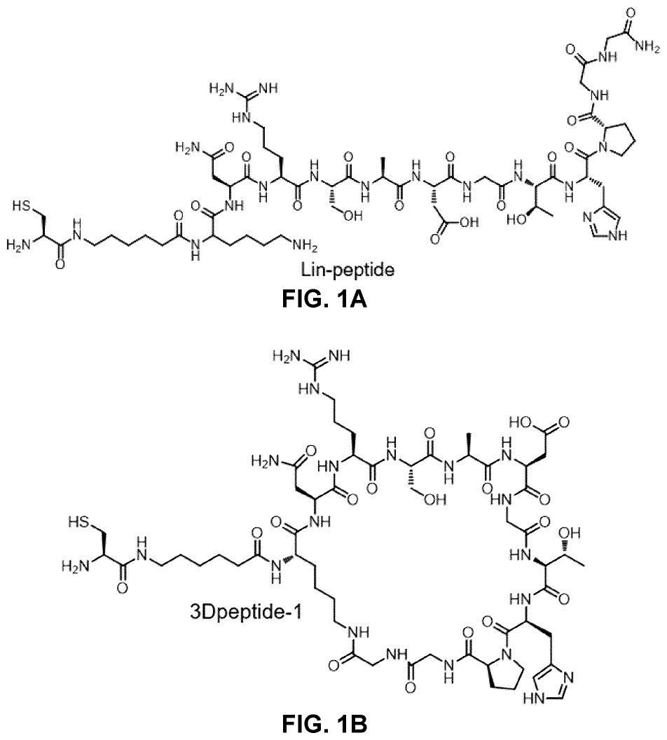

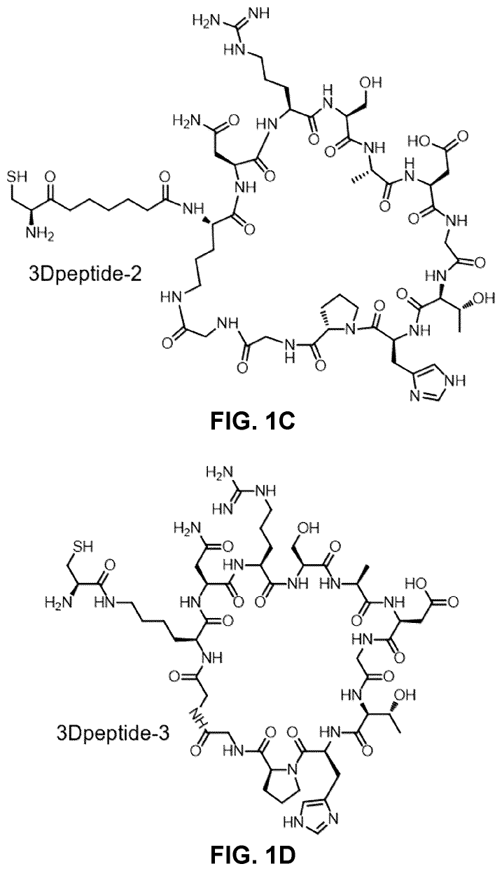

In an embodiment, the cyclic peptide has the structure depicted in any one of FIGS. 1B-D. In a further embodiment, the cyclic peptide has the structure depicted in FIG. 1D.

The cyclic peptide described herein may further comprise one or more modifications that confer additional biological properties to the cyclic peptide such as protease resistance, plasma protein binding, increased plasma half-life, intracellular penetration, etc. Such modifications include, for example, covalent attachment of molecules/moiety to the cyclic peptide (e.g., the molecule in formula II above) such as fatty acids (e.g., C₆-C₁₈), attachment of proteins such as albumin (see, e.g., U.S. Pat. No. 7,268,113); sugars/polysaccharides (glycosylation), biotinylation or PEGylation (see, e.g., U.S. Pat. Nos. 7,256,258 and 6,528,485). The cyclic peptide may also be conjugated to a molecule that increases its immunogenicity, including carrier proteins such as keyhole limpet hemocyanin (KLH), bovine serum albumin (BSA), human serum albumin (HSA) and ovalbumin (OVA), and/or polysaccharides. In an embodiment, the cyclic peptide is conjugated to a carrier protein, preferably KLH. In an embodiment, the carrier protein is conjugated via a disulfide bond to the cyclic peptide, i.e. through the sulfur of the cysteine residue of the peptide. The above description of modification of the cyclic peptide does not limit the scope of the approaches nor the possible modifications that can be engineered.

Cyclic peptides may be synthesized using methods well known in the art, for example by solid-phase synthesis as well as by conventional organic synthesis.

### Compositions Comprising the Cyclic Peptide

In another aspect, the present disclosure provides a composition comprising the cyclic peptide defined herein. In an embodiment, the composition further comprises the above-mentioned cyclic peptide and a carrier or excipient, in a further embodiment a pharmaceutically acceptable carrier or excipient, such as those described above for the antibody or an antigen-binding fragment thereof. Such compositions may be prepared in a manner well known in the pharmaceutical art, as described above for the antibody or an antigen-binding fragment thereof.

In an embodiment, the composition is an immunogenic composition or vaccine composition. Such composition may be administered by any conventional route known in the vaccine field, e.g., via a mucosal (e.g., ocular, intranasal, pulmonary, oral, gastric, intestinal, rectal, vaginal, or urinary tract) surface, via a parenteral (e.g., subcutaneous, intradermal, intramuscular, intravenous, or intraperitoneal) route, or topical administration (e.g., via a transdermal delivery system such as a patch).

In an embodiment, the composition comprising the cyclic peptide defined herein further comprises a vaccine adjuvant. The term “vaccine adjuvant” refers to a substance which, when added to an immunogenic agent such as an antigen (e.g., the cyclic peptide defined herein), non-specifically enhances or potentiates an immune response to the agent in the host upon exposure to the mixture. Suitable vaccine adjuvants are well known in the art and include, for example: (1) mineral salts (aluminum salts such as aluminum phosphate and aluminum hydroxide, calcium phosphate gels), squalene, (2) oil-based adjuvants such as oil emulsions and surfactant based formulations, e.g., incomplete or complete Freud's adjuvant, MF59 (microfluidised detergent stabilised oil-in-water emulsion), QS21 (purified saponin), AS02 [SBAS2] (oil-in-water emulsion+MPL+QS-21), (3) particulate adjuvants, e.g., virosomes (unilamellar liposomal vehicles incorporating influenza haemagglutinin), AS04 ([SBAS4] aluminum salt with MPL), ISCOMS (structured complex of saponins and lipids), polylactide co-glycolide (PLG), (4) microbial derivatives (natural and synthetic), e.g., monophosphoryl lipid A (MPL), Detox (MPL+M. Phlei cell wall skeleton), AGP [RC-529] (synthetic acylated monosaccharide), DC_Chol (lipoidal immunostimulators able to self-organize into liposomes), OM-174 (lipid A derivative), CpG motifs (synthetic oligonucleotides containing immunostimulatory CpG motifs), modified LT and CT (genetically modified bacterial toxins to provide non-toxic adjuvant effects), complete Freud's adjuvant (comprising inactivated and dried mycobacteria) (5) endogenous human immunomodulators, e.g., hGM-CSF or hIL-12 (cytokines that can be administered either as protein or plasmid encoded), Immudaptin (C3d tandem array) and/or (6) inert vehicles, such as gold particles.

### Uses of the Cyclic Peptide or Compositions Comprising Same

In another aspect, the present disclosure provides a method for inducing the production of an antibody that specifically binds to NTSR1 (e.g., to a conformational epitope located in the amino acid sequence NRSADGQHAGG (SEQ ID NO:83) from human NTSR1 and/or in the amino acid sequence NRSGDGTHPGG (SEQ ID NO:84) from rat NTSR1), in an animal, the method comprising administering to said animal an effective amount of the cyclic peptide or composition comprising same defined herein to said animal. In another aspect, the present disclosure also provides the use of the cyclic peptide or composition comprising same defined herein for inducing the production of an antibody that specifically binds to NTSR1 (e.g., to a conformational epitope located in the amino acid sequence NRSADGQHAGG from human NTSR1 and/or in the amino acid sequence NRSGDGTHPGG from rat NTSR1) in an animal. The present disclosure also provides the cyclic peptide or composition comprising same defined herein for inducing the production of an antibody that specifically binds to NTSR1 (e.g., to a conformational epitope located in the amino acid sequence NRSADGQHAGG from human NTSR1 and/or in the amino acid sequence NRSGDGTHPGG from rat NTSR1) in an animal.

In an embodiment, the above-mentioned method or use further comprises collecting the antibody produced in the animal. In a further embodiment, the above-mentioned method or use further comprises purifying the antibody collected.

The animal to which the cyclic peptide or composition comprising same is administered may be any animal conventionally used for the production of antibodies, such as a rabbit, a guinea pig, a rat, a mouse, a goat or a chicken.

In an embodiment, the animal is a chicken (hen). When using chicken (hen) as the animal host, the antibody produced in the animal are of the IgY type, and are collected (and purified) from the eggs of the animal. Methods for collecting and purifying antibodies from eggs are well known in the art (see, e.g., M. NARAT, Food Technol. Biotechnol. 41(3) 259-267 (2003) and references cited therein), and a representative method is described below.

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

The use of any and all examples, or exemplary language (e.g., “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

Unless otherwise defined, all technical and scientific terms used herein have the same meaning as commonly understood by one of ordinary skill in the art to which this invention belongs.

### MODE(S) FOR CARRYING OUT THE INVENTION

The present invention is illustrated in further details by the following non-limiting examples.

### Example 1: Materials and Methods

Peptide synthesis. The 2-chloro trityl chloride resin was recovered with the linear peptide (having a carboxylic acid function and a free amine function and the other side chains are protected). The resin was added to 5 ml DMF. 5 equimolars of DEPBT and 6 equimolars of DIPEA were added to solution. The resin was incubated for 16 h and washed with DMF and DCM. The resin was then dried under the vacuum. After the quality control for the reaction yield, the peptide cleavage was done and the purification of the macrocyclic peptide was performed by preparative HPLC.

Chicken immunization and IgY extraction. The primary immunization of KLH-conjugated peptides (Lin-peptide, 3D-peptide-1, -2 and -3) was applied in complete Freund's adjuvant, whereas following boosters were given in incomplete Freund's adjuvant. The KLH-conjugated peptides were injected intramuscularly. After three months of immunization with monthly antigen boost, all the eggs were pooled according to antigen and time of collection. Egg yolk was separated from the white and proteins were extracted as previously described with some modifications (“Production and Application” R. Schade et al. 2000, Springer-Verlag, Lab Manuals, ISBN 3-540-66679-6. “Isolation and Purification of Chicken Egg Yolk immunoglobulins: A review”). All extracted IgY (IgY-Lin, IgY-3D-1, IgY-3D-2 and IgY-3D-3) were processed for a second step of immunoaffinity purification using the corresponding peptide conjugated to agarose beads.

Antibody sequencing. The sequences of the antibodies were determined by Next-generation sequencing (NGS) and confirmed by Sanger sequencing.

Cells and reagents. Human embryonic kidney (HEK) 293A and 293T cells were maintained in Dulbecco's modified Eagle's medium (DMEM) supplemented with 10% fetal bovine serum (FBS) and antibiotics at 37° C., 5% CO₂. Chinese hamster ovary (CHO)-K1 cells were obtained from the American Type Culture Collection (ATCC) and were maintained in DMEM/F12 supplemented with FBS 10%, L-glutamine, HEPES and antibiotics at 37° C., 5% CO₂. All cell culture reagents were from HyClone (Thermo Scientific, Logan, Utah, USA).

HEK293A and CHO-K1 cells were transformed with a lentivirus-based system to stably express the rat NTS1 receptor. To generate the lentiviral vector expressing HA-tagged rat NTS1, the 2HA-rNTS1 coding sequence was amplified from a custom plasmid (p2HA-rNTS1) with following primers (forward: 5′-TGCATCTAGAGCCACCATGTACCCATACGA-3′ (SEQ ID NO:122) and reverse: 5′-TCACTGCAGAATTCCTAGTACAGGGTCTCC-3′ (SEQ ID NO:123)) and cloned into pCDH-MCS-Ef1-puro cDNA cloning and expression vector (System Biosciences, Mountain View, Calif., USA) using Xbal and EcoRl restriction enzymes (New England Biolab, Massachusetts, USA). HEK293T cells were then transiently co-transfected with the pCDH lentivector, pMD2.G (gift from Didier Trono, Addgene plasmid #12259) and psPAX2 (gift from Didier Trono, Addgene plasmid #12260) using polyethylenimine (MW 25,000; Polysciences, Warrington, Pa.) as transfection reagent. The viral particles were then collected 48 h post-transfection, filtered (0.45 μM PVDF syringe filter, ThermoFisher Scientific, Massachusetts, USA) and used to generate the cells stably expressing HA-ratNTS1. HEK and CHO-K1 cells expressing pCDH-CMV-2HA-rNTS1-Ef1-puro were selected using puromycin at concentrations of 0.5 μg/ml and 8 μg/ml, respectively.

Enzyme-linked immunosorbent assays on peptides. All tested peptides (Lin-peptide, 3Dpeptide-1, 3Dpeptide-2 and 3Dpeptide-3) were plate-coated at a concentration of 3.5 μmole per well in a Well-Coated™ sulfhydryl binding, 8-well strip plate, clear (G-Biosciences, MO 63132-1429, USA) with a binding buffer (0.1M Na₂HPO₄, 0.15M NaCl, 10 mM EDTA pH 7.2) overnight at 4° C. To block, L-cysteine (1 μM in binding buffer) was added to each well, 1 h at room temperature (RT). Tested antibodies (IgY-Lin, IgY-3D-1, IgY-3D-2 and IgY-3D-3) were diluted at a final concentration of 100 ng/ml (PBS 0.25% Tween 20, 0.3% milk) and pre-incubated with increasing amounts (from 0.3 nM to 10 000 nM) of its corresponding free-peptide or a fixed concentration (10 μM) of either free Lin-peptide, 3D-peptide-1, -2 or -3. All IgY-peptide mixes and the controls (IgY only as the positive control and secondary antibody only as the negative control) were added to each peptide-coated well and incubated for 2 h at RT. Four washes with PBS 0.25% Tween® 20 (PBST) were performed and the secondary antibody (alpaca anti-chicken HRP, Immune Biosolutions, Sherbrooke, QC, CA) was added for 45 minutes at RT. After four additional washes with PBST, 3, 3′, 5, 5′-Tetramethylbenzidine (TMB) (Sigma-Aldrich, St-Louis, Mo., USA) was added and incubated for 15 minutes at RT before the reaction was stopped with HCl 2N. Absorbance at 450 nm was read with a Tecan GENios® plate reader (Tecan Austria GmbH, Austria).

Immunofluorescence microscopy. 120,000 HEK293A cells stably expressing HA-rNTS1 or transiently expressing either HA-tagged-rNTS1, -rNTS2 or -rAPJ were seeded on microscope glass coverslips in 6-well plates. The next day, cells were washed with PBS, fixed for 10 min in 3.7% formaldehyde-PBS at 37° C., washed three times in PBS and permeabilized with 0.1% Triton X-100 for 2 min on ice. Cells were then washed twice with PBS and treated with 10% normal goat serum (NGS) in PBS for 1 h at RT. This saturation step was followed by an overnight incubation with a mouse monoclonal HA-probe antibody (1:200, (F-7) sc7392, Santa Cruz Biotechnology, CA, USA) and 5 μg/ml of each chicken polyclonal anti-NTS1 (Lin, 3D-1, 3D-2 and 3D-3) or a mix containing the antibody and the corresponding peptide at a ratio of 1:10 (IgY: peptide) in PBS NGS 10%. Fluorescent staining was done by incubating with an Alexa Fluor 488-conjugated goat anti-chicken antibody and an Alexa Fluor™ 647-conjugated goat anti-mouse antibody (each used at 1:200 dilution). Cells were washed 4 times in PBS before mounting in ProLong® gold antifade mountant with DAPI (ThermoFisher scientific, Massachusetts, USA). Pictures were generated using a confocal Olympus IX81 FV1000 LSM microscope and the Fluoview® FV10-ASW 3.1 viewer software. Imaging parameters were set to identical values across samples. Colocalization analysis and coefficient calculations (Pearson and Overlap 488/647) were performed on z-stack images (10 slices per field) of five randomly chosen fields per condition (FV10-ASW 3.1 viewer software).

Tissue fixation and immunohistochemistry. Adult male rats were anesthetized with 5% isoflurane and transcardially perfused with 4% paraformaldehyde (PFA) in phosphate buffer (0.1M). Brains were cryoprotected overnight (phosphate buffer 0.1M, 30% sucrose) at 4° C. and frozen in isopentane at −40° C. for 1 min. Coronal sections were cut on a freezing microtome and collected in PBS to proceed with immunostaining. Free-floating brain sections were washed 4 times with PBS and incubated with PBS-H₂O₂ 3% for 1 h. Sections were washed 4 additional times with PBS, permeabilized and blocked with a blocking solution (PBS, NGS 3% and triton-X 0.3%) for 1 h. An incubation of 2 hours was done with the chicken antibodies (IgY-Lin, IgY-3D-1/2 and 3) at a concentration of 5 μg/ml in a dilution solution (PBS, NGS 1% and Triton-X 0.3%). After washes, the goat anti-chicken HRP-conjugated antibody was added (1:200) in the dilution solution and incubated for 1 h, and washed again. Solutions A (1:100) and B (1:100) were diluted in the dilution solution, added to the slides and incubated for 1 h. After washes, DAB solution (PBS, diaminobenzidine 0.05%, H₂O₂ 0.015%) was added for 3 min and reaction was stopped with PBS. All steps were performed at room temperature under agitation. The brain sections were dried and coverslipped with Permount® Mounting Media (SP15-500, Fisher Scientific, NY, USA). Images were acquired with a Leica DM4000 microscope at a 10× magnification.

Western blots. HEK293A cells were transiently transfected with equal amount (6 μg) of either HA-tagged-rNTS1, -rNTS2, -rAPJ or the empty vector as a negative control. Whole cells were lysed in a reducing lysis buffer (280 mM NaCl, 50 mM Tris, 0.015 g DTT, 0.5% NP-40, pH 8.0) ora native lysis buffer (280 mM NaCl, 50 mM Tris, 0.5% sodium deoxycholate (NaDOC), 1% n-dodecyl β-D-maltoside (DDM)) supplemented with complete protease inhibitor cocktail (Roche, Bale, Switzerland) and sonicated 30 sec. 48 h post transfection. Whole cell lysates were further processed for immunoprecipitation and/or polyacrylamide gel electrophoresis (PAGE). Regarding the last one, lysates were heated at 37° C. in denaturating/reducing protein sample buffer (60 mM Tris-HCl pH 6.8, 10% glycerol, 0.002% bromophenol blue, 2% SDS, 2% 2-mercaptoethanol) or native protein sample buffer (31.25 mM Tris-HCl pH 6.8, 25% glycerol, 0.005% bromophenol blue). The protein samples (20 μg of total protein per well) were loaded and resolved by SDS or native PAGE. After transfer to nitrocellulose membranes, blots were probed with the different chicken antibodies (IgY-Lin, IgY-3D-1, IgY-3D-2 and IgY-3D-3) at a concentration of 1 μg/ml or a chicken anti-HA antibody (1:1000, Immune Biosolutions Inc, Sherbrooke, QC, CA), followed by a secondary antibody, goat anti-chicken HRP (1:5000, Immune Biosolutions Inc, Sherbrooke, QC, CA). A rat monoclonal anti-HA high affinity HRP-conjugated antibody (1:1000, Roche, Sigma-Aldrich, St-Louis, Mo., USA) was used to detect the protein expression levels of the whole-cell lysates in the selectivity assay. All blots were revealed using a chemiluminescence detection system (Bio-Rad clarity western ECL, Bio-Rad, Hercules, Calif., USA). Images were recorded on a Chemidoc® MP using the software Image Lab version 5.0.

Immunoprecipitations. All the chicken antibodies tested (IgY-Lin, IgY-3D-1, IgY-3D-2, IgY-3D-3 and IgY anti-HA) were conjugated, in equivalent molar ratio, to maleimide magnetic beads (Immune Biosolution Inc.). The antibody-coupled magnetic beads (10 μl) were added to each sample (200 μl of whole-cell lysate at 1 mg/ml of total protein) and to negative controls which consisted of lysate of cells transfected with the empty vector or of samples containing the targeted receptor with a mix of pre-incubated IgY-peptide in a 1:10 ratio in the assay preceding the mass spectrometry. All samples were incubated on a rotator at 4° C. overnight. The next day, protein-antibody-magnetic beads complexes were washed with PBS Tween® 0.01% four times followed by 4 washes with PBS without Tween in the case of samples tested in mass spectrometry. Immunoprecipitated proteins were eluted with elution buffer (ammonium hydroxide 0.5M) and processed for Western blot as described above or snap-freezed and further lyophilized (program: −40° C. for 6 h; −21° C. for 10 h; 20° C. for 4 h) using a stoppering tray dryer (Labconco, Kansas City, Mo., USA). The samples were sent to two platforms to assess mass spectrometry: PhenoSwitch (Sherbrooke, QC, CA) and the proteomic platform of the research center of CHUL (Laval University, QC, CA).

Mass spectrometry and protein identification. Proteins were reconstituted in 100 μl of 50 mM Tris pH 8+0.75 M Urea, reduced with 10 mM DTT for 15 min at 65° C. and alkylated with 15 mM iodoacetamide for 1 h at RT in the dark. Trypsin/LysC (1 μg) was added to each sample and proteins were digested overnight at 37° C. with agitation. Digestion was stopped by the addition of 2% formic acid and peptides were purified by reversed phase SPE. Acquisitions were performed with an ABSciex TripleTOF 5600 (ABSciex, Foster City, Calif., USA) and a Thermo Orbitrap® Fusion Tribrid (Thermo Scientific, Logan, Utah, USA) by Phenoswitch and the Proteomic Platform, respectively. Protein identification from Phenoswitch was performed with ProteinPilot V4.5 beta (ABSciex) with the instrument pre-set for TripleTof5600, Iodoacetamide as cystein alkylation as special factor. Thorough search with false discovery rate analysis was performed with biological modification emphasis against the rat (Rattus norvegicus) proteome (with custom proteins sequence added). For protein identification and data analysis global false discovery rate (FDR) was set at 1% and local false discovery rate was set at 5%. Peptides for the Neurotensin receptor 1 were reviewed manually to ensure that they were meeting a basic quality standard. The Proteomic Platform analyzed all MS/MS samples using Mascot (Matrix Science, London, UK; version 2.5.1). Mascot was set up to search the CP_RattusNorvegicus_10116_CO_20161019 database (unknown version, 31602 entries) assuming the digestion enzyme trypsin. Mascot was searched with a fragment ion mass tolerance of 0.6 Da and a parent ion tolerance of 10 PPM. Carbamidomethyl of cysteine was specified in Mascot as a fixed modification. Deamidated of asparagine and glutamine and oxidation of methionine were specified in Mascot as variable modifications. Scaffold (version Scaffold_4.7.5, Proteome Software Inc., Portland, Oreg.) was used to validate MS/MS based peptide and protein identifications. Peptide identifications were accepted if they could be established at greater than 99% probability to achieve an FDR less than 1% by the Scaffold Local FDR algorithm. Protein identifications were accepted if they could be established at greater than 99% probability to achieve an FDR less than 1% and contained at least 1 identified peptide.

Cyclic AMP production measurement. CHO-K1 cells stably expressing the HA-rNTS1 were used to perform this competitive immunoassay based on the HTRF technology (cAMP-Gs dynamic kit, Cisbio US, Bedford, Mass., USA). Cells (2000/well) were either stimulated with NT 8-13 (6 nM (EC₅₀) and 10 μM (ECₘₐₓ) only, or a mix of NT 8-13 (6 nM) and each IgY (-Lin, -3D-1, -3D-2 and -3D-3) for 30 min at 37° C. in an Alpha-plate 384 SW (PerkinElmer, Waltham, Mass., USA). Not stimulated cells were used as negative control. Reagents were added as described in the manufacturer protocol. Fluorescence was measured at 620 nm and 665 nm with a Tecan GENios plate reader (Tecan Austria GmbH, Austria).

Binding assay. HEK293 cells expressing the HA-tagged rat NTS1 (HA-rNTS1) were frozen when they reached 80% confluency. Cells were scrapped-off the dish with 10 mM Tris, 1 mM EDTA, pH 7.5 and centrifuged at 15,000 g for 5 min at 4° C. The pellet was then re-suspended in binding buffer. Competitive radioligand binding experiments were performed by incubating 15 μg of cell membranes expressing the HA-rNTS1 receptor with 45 μM of ¹²⁵I-[Tyr³]-NT (2200 Ci/mmol) in binding buffer (50 mM Tris-HCl, pH7.5, 0.2% BSA) in the presence of increasing concentrations of chicken antibodies (IgY-Lin, IgY-3D-1, IgY-3D-2 and IgY-3D-3) for 60 min at 25° C. After incubation, the binding reaction mixture was transferred in polyethylenimine-coated 96-well filter plates (glass fiber filters GF/B, Millipore, Billerica, Mass.). Reaction was terminated by filtration, and plates were washed three times with 200 μL of ice-cold binding buffer. Glass filters were then counted in a γ-counter (2470 Wizard2, PerkinElmer). Non-specific binding was measured in the presence of 10⁻⁵ M unlabeled NT[8-13] and represented less than 5% of total binding. IC₅₀ values were determined from the competition curves as the unlabeled ligand concentration inhibiting half of the ¹²⁵I-[Tyr³]-NT-specific binding.

Bioluminescence resonance energy transfer (BRET)-based biosensor assays. All BRET assays were performed at Domain Therapeutics NA Inc (Montreal, QC, Canada). Experiments were all performed in HEK 293T cells transiently expressing the HA-rat NTS1 receptor and processed as previously described with minor modifications (Rives et al., Mol Pharmacol., 2018). Cells were plated in 384-well plates. First, agonist mode was tested by treating cells, 48 h post-transfection, with increasing amounts of NT 1-13 (0.1 μM to 0.1 mM), SR48692 (0.1 μM to 0.1 mM) and chicken antibodies: IgY-Lin, IgY-3D-1 and IgY-3D-3 (0.02 μM to 2 μM). Cells were washed with Tyrode-HEPES buffer (Sigma, Cat #T2145-H9136) and proceed for an equilibration period of 60 min at room temperature. Then, e-Coelenterazine Prolume® Purple (1.8 μM, Methoxy e-CTZ; Nanolight, Cat #369) was added to each well and a first read was acquired on a Synergy NEO plate reader. For the antagonist mode, an EC50 of NT 1-13 was added to the previous wells, which were treated with SR48692 and antibodies, and a second read was performed after an incubation of 10 min. The BRET signal was calculated as the ratio of acceptor emission to donor emission.

Preparation of humanized and recombinant antibodies. gBlocks containing humanized framework scFv sequences were synthesis by Integrated DNA Technologies, Inc. Upon reception, gBlocks were resuspended in 10 mM Tris pH 8.0 and introduced in pTT5-Fc plasmid by the Gibson Assembly method. Assembled products were transformed into DH5u competent cells and were grown on selection plates overnight. Colonies were counted and validated by colony PCR. Plasmid were isolated by Minipreps for positive clones and sent for Sanger Sequencing for further analysis. Plasmids containing confirmed positive clones were amplified and isolated by Maxipreps. Plasmids were transfected in CHO-3E7 and cells are maintained for 7 days. Culture medium was collected and antibodies were isolated by affinity chromatography using protein A column.

### Example 2: Description of Peptides and Evaluation of Antibody Selectivity and Affinity for Peptides

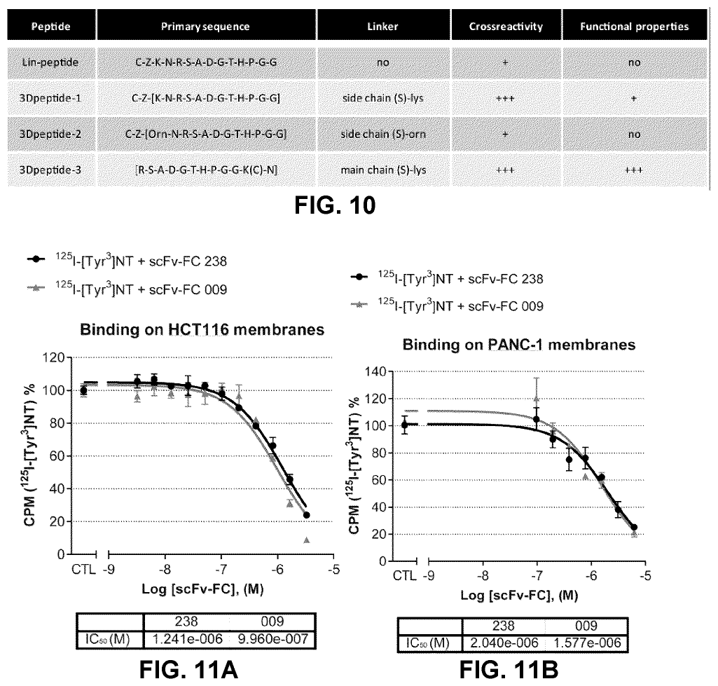

The peptide selected for this study, which served as the antigenic determinant, are based on an 11-amino acid sequence of the second extracellular loop of the agonist-bound crystallized rat NTS1 receptor. The structure and description of the four designed peptides (Lin-peptide, 3Dpeptide-1, -2 and -3) and linkers are presented in FIGS. 1A-D and FIG. 10. Following immunization with the four peptides, chicken polyclonal antibodies (IgY) were extracted and immunopurified to generate four distinct antibodies: IgY-Lin, IgY-3D-1, -2 and -3. Resulting antibody cross-reactivity and functional properties, based on all validation results described below, are indicated in FIG. 10.

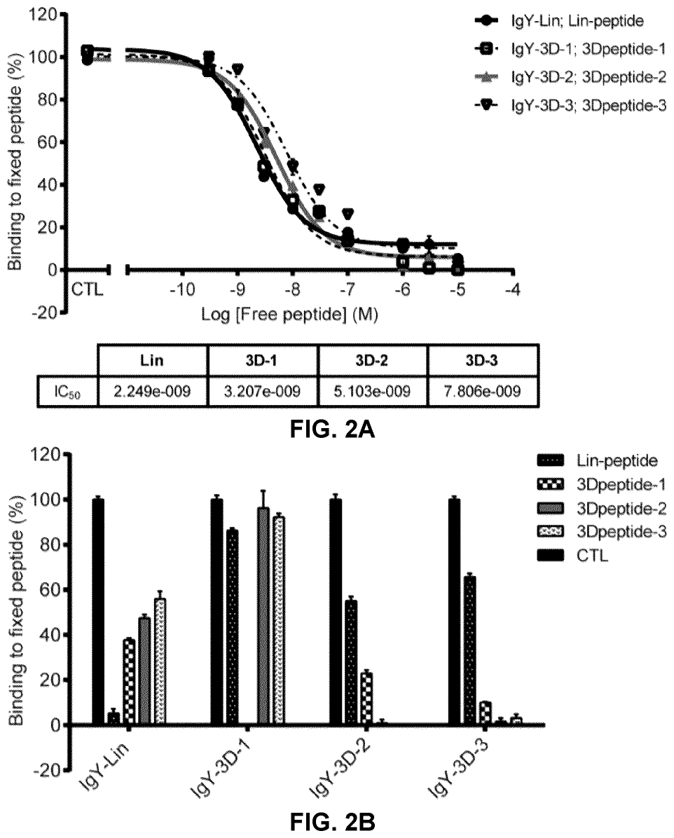

To validate the epitope binding efficacy, all antibodies were tested in an enzyme-linked immunosorbance assay (ELISA) on fixed peptides (FIG. 2A). Inhibition curves were generated by pre-incubating the antibody with an increasing amount of its matching free-peptide (e.g., IgY-Lin with the Lin-peptide) to block the fixed peptide detection. Absorbance measures at 450 nm of the controls containing only the antibody without free-peptide were normalized to a total detection of a 100% and all samples were normalized in percentage to their corresponding control. To inhibit 50% of the binding to fixed peptide, calculated concentrations (IC₅₀) were comparable for the four antibodies, ranging from about 2.25 nM (Lin) to about 7.81 nM (3D-3).

To analyse the selectivity of the antibodies for the designed peptides, a competition assay was conducted by mixing the indicated antibody (FIG. 2B) with a fixed concentration (10 μM) of each peptide. The control corresponded to the antibody only, without free-peptide. All absorbance measures were transformed into percentage and normalized to the respective control. The results obtained show that all antibodies were blocked by their matching peptide. The IgY-Lin was partially blocked by the three 3D-peptides (up to 67% blockade). Interestingly, the IgY-3D-1 was only efficiently blocked by its corresponding peptide (3Dpeptide-1). IgY-3D-2 and -3 had similar phenotypes and were blocked by all 3D-peptides, the 3Dpeptide-1 to a lesser extent, and were only partially blocked by the Lin-peptide (up to 45% blockade).

### Example 3: Validation of Antibodies—Detection of the Rat NTS1 Receptor

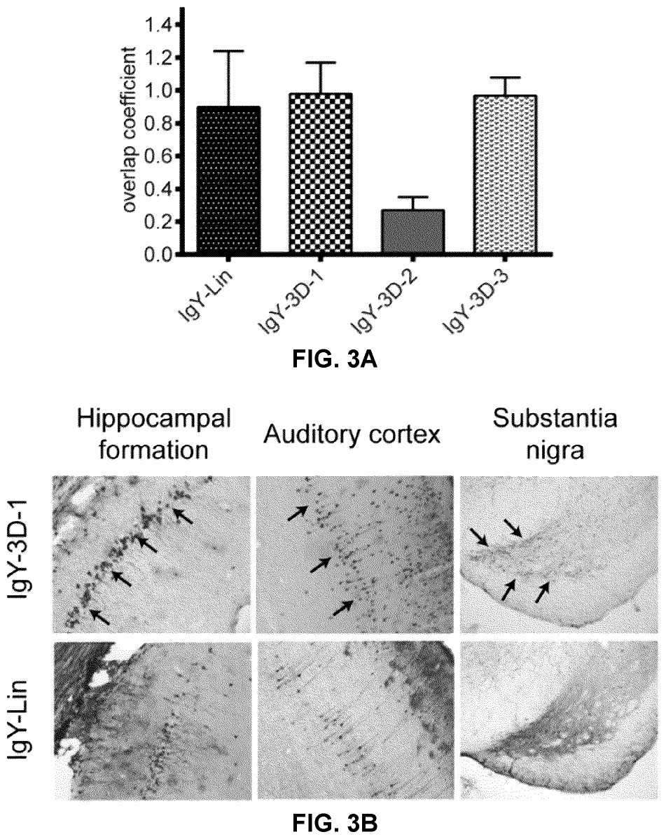

The antibodies were validated for their ability to detect the epitope with which they were produced; the next step was to determine if they were able to detect the complete protein. Immunofluoresence (IF) microscopy was performed on fixed HEK293A cells stably expressing the HA-tagged rat NTS1 receptor and a colocalization analysis was performed: the overlap coefficient was calculated based on the signal generated by the chicken antibodies (Alexa Fluor™ 488) on the signal generated by the HA (Alexa Fluor™ 647). The graph in FIG. 3A shows the calculated overlap coefficient (488/647) of ten z-stack slices (0.5 μm), considered as replicates, from five randomly chosen fields per condition. According to the literature, an overlap coefficient below 0.5 is not considered as a significant colocalization, as is the case with the IgY-3D-2.

To validate the detection of the endogenous rat NTS1 receptor, immunohistochemistry (IHC) was performed on rat brain sections with the chicken antibodies. The results obtained with the IgY-Lin and IgY-3D-1 are pictured in FIG. 3B. Regions previously described as highly expressing the rNTS1 receptor are presented and arrows are indicating the NTS1 specific signal. IgY-3D-1 and IgY-3D-3 were efficiently detecting the rat NTS1 receptor without generating non-specific signal as obtained with IgY-Lin. IgY-3D-2 was not able to detect the receptor in the rat brain.

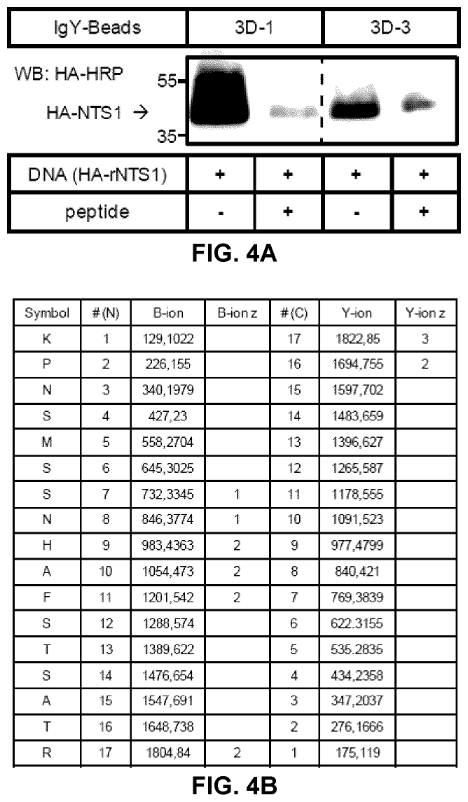

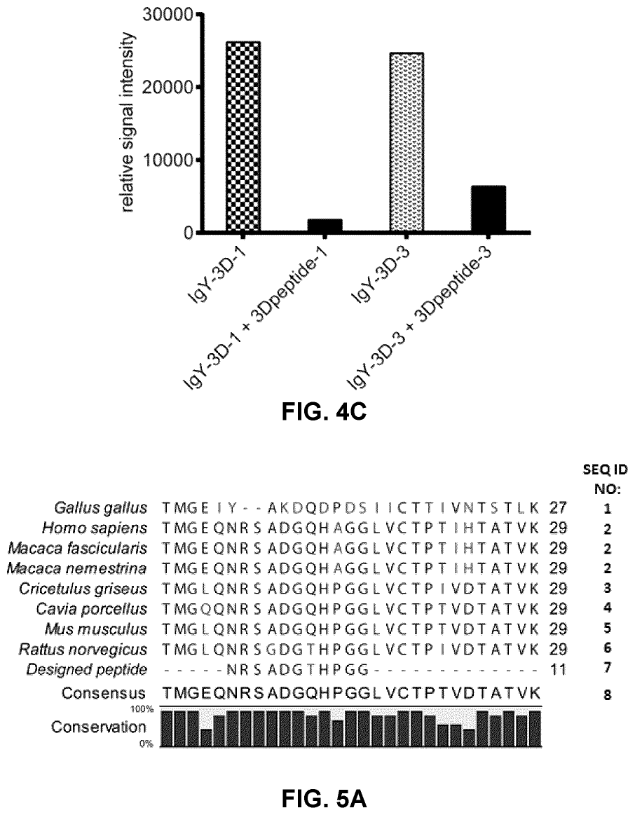

A demonstration of the specific interaction of the antibodies with the rat NTS1 receptor was established by mass spectrometry identification. First, the rat NTS1 receptor was isolated by immunoprecipitation (IP) with the chicken antibodies (IgY-3D-1 and -3) conjugated to magnetic beads, from HEK293A cells overexpressing HA-rNTS1. The negative controls used corresponded to a pre-incubation of the IgY-conjugated beads with their matching peptide in a 1:10 ratio. The immunoblot was revealed with an anti-HA-HRP antibody (FIG. 4A). All samples were sent for analysis and a peptide from the Rattus norvegicus was clearly identified and corresponded to the C-terminus domain of the rat NTS1 receptor. The identified peptide sequence and its ionic profile are shown in FIG. 4B. The interaction between NTS1-specific antibodies and the NTS1 receptor was significantly blocked by the pre-incubation with the corresponding peptide as shown by the graph in FIG. 4C.

### Example 4: NTSR1 Conservation Among Species and Antibodies Selectivity

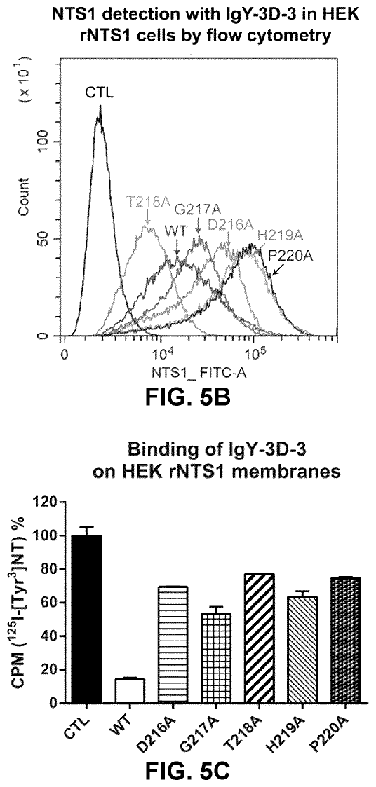

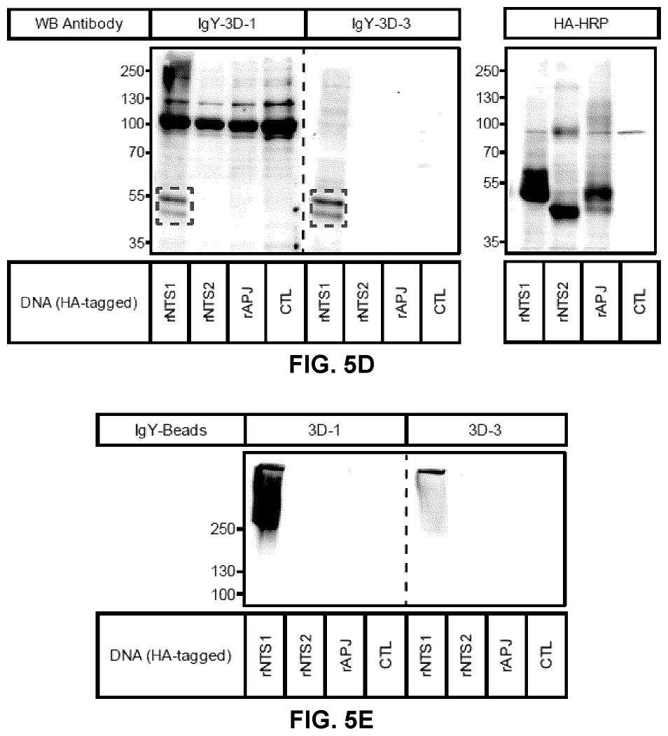

The second extracellular loop rat NTS1 receptor X-ray based peptide sequence, on which the immunogens used were based, is largely conserved among species, since one and two amino acids are differing from mouse and human NTS1 protein sequences, respectively (FIG. 5A). NTS1 receptor antibodies (IgY-3D-1 and IgY-3D-3) raised against this epitope were shown to recognize the human NTS1 receptor and are expected to recognize the mouse receptor. The results depicted in FIG. 5B shows that the polyclonal IgY-3D-3 antibody binds to NTS1 protein mutated (alanine substitution) at the five amino acids of the designed epitope (D216, G217, T218, H219 and P220) predicted to be the most exposed, with stronger binding (relative to native NT) for some of the mutants. The IgY-3D-3 antibody was also able to induce displacement of radiolabeled neurotensin (NT) on the mutants, although at lower levels relative to native NT (FIG. 5C).

Target-selectivity is determinant in the production of antibodies and had to be evaluated. To address this question, all antibodies were tested for their ability to detect other class A GPCRs: the neurotensin low-affinity receptor 2 (rNTS2) and the apelin receptor (rAPJ), both from rat. HEK293A cells overexpressing either the HA-tagged-rNTS1, rNTS2, rAPJ or an empty vector control were processed for Western blots (FIG. 5D) and native immunoprecipitations (FIG. 5E). The results depicted in these figures show that NTS1-antibodies IgY-3D-1 and IgY-3D-3 only detected the rat NTS1 receptor.

### Example 5: Functional Properties of Antibodies

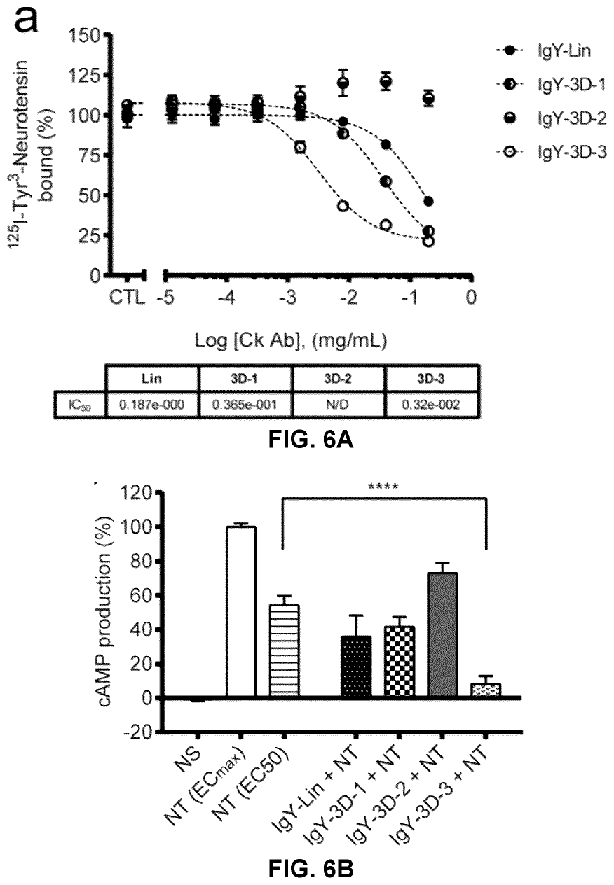

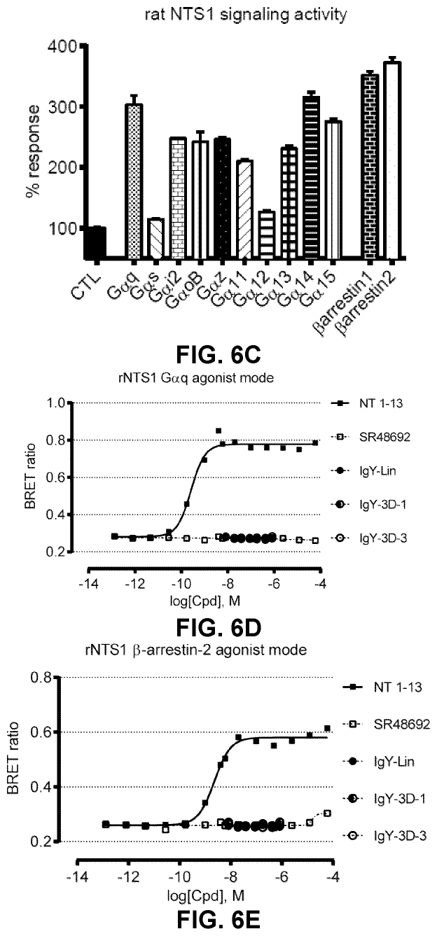

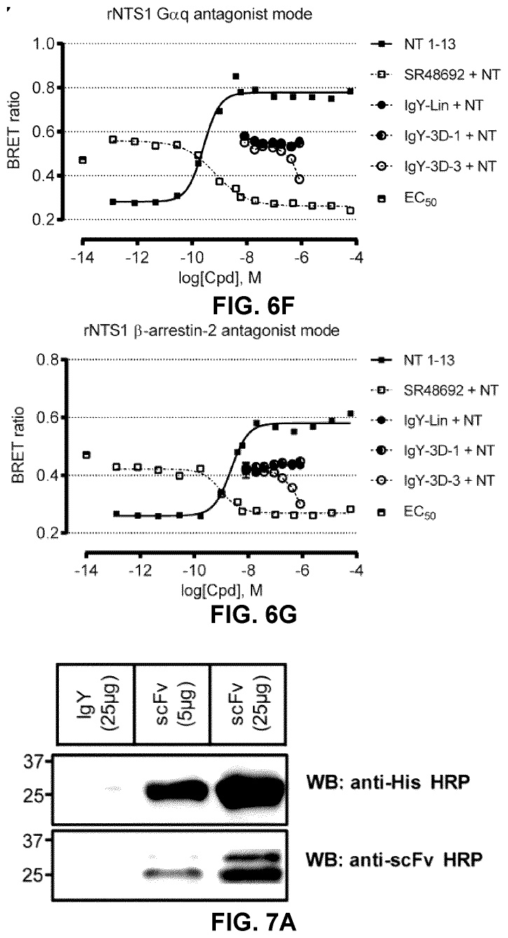

A robust technique to validate the functional properties of a compound or an antibody on the NTS1 receptor is to measure the displacement of the radiolabeled-neurotensin (¹²⁵I-Tyr³-Neurotensin) binding to its receptor by adding the antibody in a dose-response manner (FIG. 6A). IgY-3D-3 was the most potent antibody and was capable of efficiently displacing NT at low concentrations (IC₅₀=3.2 μg/ml). IgY-3D-1 and IgY-Lin were shown to displace 50% of the neurotensin at higher concentrations relative to IgY-3D-3 (36.5 μg/ml and 187 μg/ml, respectively). IgY-3D-2 did not significantly change the level of bound NT.

NTS1 receptor activation following the neurotensin binding triggers multiple signaling pathways and the first tested was the accumulation of cyclic AMP (cAMP) in response to Gs-coupled NTS1 activation. The amount of cAMP produced was calculated in CHO-K1 cells stably expressing the rat NTS1 receptor with an assay based on Homogeneous Time Resolved Fluorescence (HTRF) technology. Cells were stimulated with NT (at a concentration corresponding to the EC₅₀) and incubated with antibodies to evaluate their capacity to block the production of cAMP (FIG. 6B). The IgY-3D-3 antibody was the only antibody to significantly inhibit the production of cAMP induced by NT under these conditions.

Then, the rat NTS1 signaling activity profile following NT stimulation was determined in HEK293 cells transiently expressing HA-rNTS1 using a BRET-based assay and all signaling pathways tested are indicated in the graph (FIG. 6C). G_{αq} and βarrestin2 generated good responses to NT stimulation and were subsequently tested in a concentration-response assay with the chicken antibodies (IgY-Lin, IgY-3D-1 and IgY-3D-3) in an agonist mode (FIG. 6D, 6E) and antagonist mode (FIG. 6F, 6G). None of the antibodies have been shown to be effective for the activation of all tested signaling pathways (agonist mode). In antagonist mode, cells were stimulated with NT (EC₅₀) and incubated with antibodies to assess their capacity to block G_{αq}- and βarrestin2-dependent signaling. IgY-3D-3 was the only antibody capable of blocking these signaling pathways. The known NTS1 antagonist SR48692 was used as a positive control.

### Example 6: NTSR1 Specific Recombinant Antibodies and Functional Properties

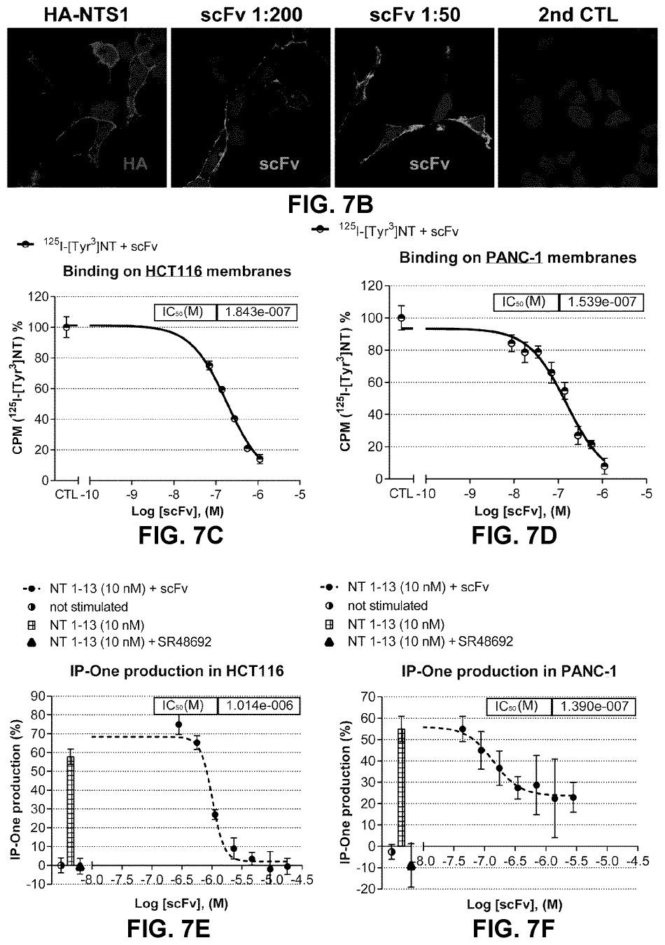

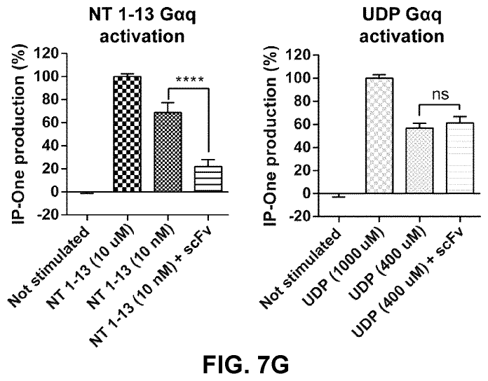

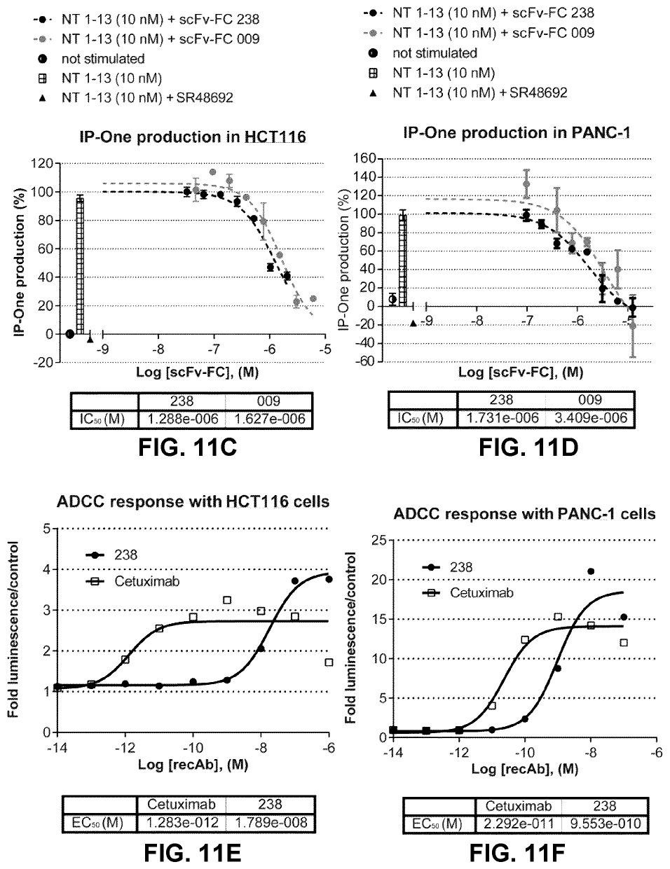

Phage display was performed using the genetic material of the chicken immunized with the 3Dpeptide-3 to generate the scFv library; relevant sequences were selected to construct a chicken His-tagged scFv (scFv 238) and a chicken scFv with the Fc fragment from human IgG (scFv-Fc 238). A Western blot confirming the size (˜25 kDa) of the scFv is presented in FIG. 7A and was done with two HRP-conjugated antibodies: chicken anti-His and anti-scFv. A second chicken scFv with the Fc fragment from human IgG was also prepared (scFv-Fc 009, see FIG. 8H for the sequence of scFv 009). scFv 238 was also able to detect HEK cells stably expressing HA-human NTS1 (FIG. 7B), and to displace radiolabeled-neurotensin on HCT116 and PANC-1 membranes preparations (FIGS. 7C and 7D). Similar results were obtained with scFv-Fc 238 and scFv-Fc 009, as shown in FIGS. 11A and 11B.

To test the functional properties of the recombinant antibodies, the accumulation of inositol monophosphate (IP-One) in response to G_{q}-coupled NTS1 activation was measured in a concentration-response assay using increasing amount of the scFv 238 (FIG. 7E) and scFv-Fc 238. HCT116 cells, which are colorectal cancer cells endogenously expressing the human NTS1 receptor, were stimulated with NT (10 nM) and incubated with the recombinant antibodies. Both scFv 238 and scFv-Fc 238 were able to inhibit NT-induced IP-One production in HCT116 cells, with a complete (˜% 100) or ˜50% inhibition of IP-One production at a concentration of 100 ng/ml for scFv 238 and scFv-Fc 238, respectively. Similar results were obtained with scFv 238 on PANC-1 cells, which are human pancreatic cancer cells expressing the human NTS1 receptor (FIG. 7F), and with scFv-Fc 238 and another scFv-Fc (scFv-Fc 009) (FIGS. 11C and 11D). scFv 238 was shown to inhibit IP-one production following activation of the G_{αq} pathway with NT 1-13, but not with UDP, in HCT116 live cells, confirming the specificity of the antibody for NTS1.

The results depicted in FIGS. 11E and 11F show that scFv-Fc 238 has the ability to trigger an ADCC response against HCT116 and PANC-1 cells. Thus, in addition to blocking NT-mediated signaling in tumor cells, the recombinant antibody is able to induce the killing of the tumor cells.

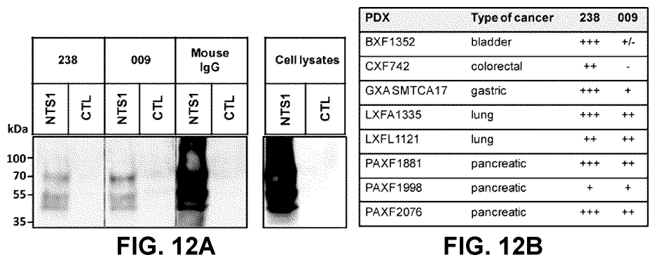

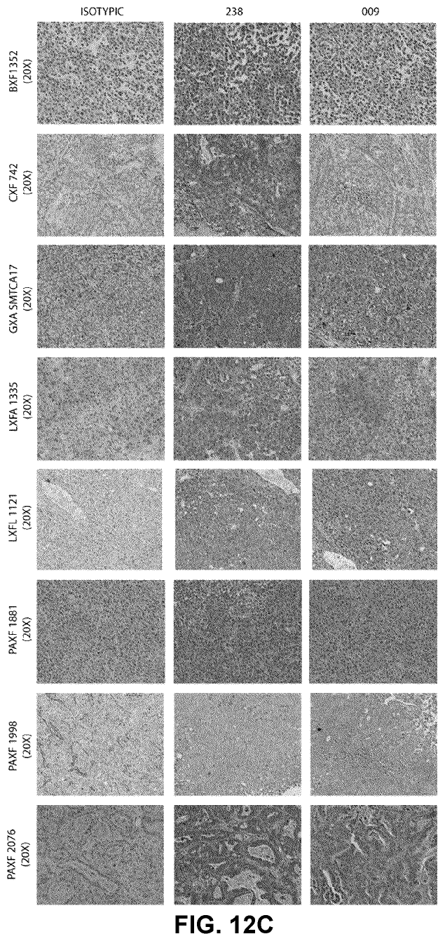

The results presented in FIGS. 12A-12C show that scFv-Fc 238 and/or scFv-Fc 009 can detect human NTS1 by IHC on stably-transfected HEK cells (FIG. 12A), as well as on several cancer patient-derived xenografts from different origins, including bladder, colorectal, gastric, lung and pancreatic cancer xenografts (FIGS. 12B, 12C), providing evidence that the recombinant antibodies may be useful to target NTS1-expressing tumors of any origin.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. (canceled)

2. An antibody or an antigen binding fragment thereof, which specifically binds to a conformational epitope located in the amino acid sequence NRSADGQHAGG (SEQ ID NO:83) from human Neurotensin receptor type 1 (NTSR1) and/or in the amino acid sequence NRSGDGTHPGG (SEQ ID NO:84) from rat NTSR1.

3. (canceled)

4. The antibody or antigen binding fragment thereof according to claim 2, which is a monoclonal antibody or an antigen binding fragment thereof.

5. The antibody or antigen binding fragment thereof according to claim 2, which comprises the following combination of complementarity determining regions (CDRs): a light chain CDR1 (CDR-L1) comprising an amino acid sequence having at least 80% identity with the sequence SGGGSTDAGSYYYG (SEQ ID NO:9) or SGSGSSWHGYG (SEQ ID NO: 125); a CDR-L2 comprising an amino acid sequence having at least 80% identity with the sequence FNDKRPS (SEQ ID NO:10) or DNTNRPS (SEQ ID NO:126); a CDR-L3 comprising an amino acid sequence having at least 80% identity with the sequence GSADSTGGI (SEQ ID NO:11) or GGYDSSTYAGI (SEQ ID NO: 127); a heavy chain CDR1 (CDR-H1) comprising an amino acid sequence having at least 80% identity with the sequence GFTFSSFNMF (SEQ ID NO:12) or GFTFSSFNMI (SEQ ID NO: 128); a CDR-H2 comprising an amino acid sequence having at least 80% identity with the sequence AQIIDGAGSRTAYGAAVKG (SEQ ID NO:13) or ASICTGGSYTYYAPAVKG (SEQ ID NO:129); and a CDR-H3 comprising an amino acid sequence having at least 80% identity with the sequence GGHYWGGASIDA (SEQ ID NO:14) or SVDGVGWHAGQIDA (SEQ ID NO:130).

6-7. (canceled)

8. The antibody or antigen binding fragment thereof according to claim 5, wherein (i) the CDR-L1 comprises the sequence SGGGSTDAGSYYYG (SEQ ID NO:9), SGGGSTDASSYYYG (SEQ ID NO:19) or SGSGSSWHGYG (SEQ ID NO: 125), (ii) the CDR-L2 comprises the sequence FNDKRPS (SEQ ID NO:10) or DNTNRPS (SEQ ID NO:126), (iii) the CDR-L3 comprises the sequence GSADSTGGI (SEQ ID NO:11) or GGYDSSTYAGI (SEQ ID NO: 127), (iv) the CDR-H1 comprises the sequence GFTFSSFNMF (SEQ ID NO:12), GFTFSSFNRF (SEQ ID NO:15), GFTVSSFNMF (SEQ ID NO:16), GFTFSSFNMC (SEQ ID NO:17), GFTFSSFNMV (SEQ ID NO:18), GFTFSGFNMF (SEQ ID NO:20), GFTFRSFNMF (SEQ ID NO:21), GFTFSRFNMF (SEQ ID NO:23), GVTFSSFNMF (SEQ ID NO:24), GFTFSSVNMF (SEQ ID NO:25), GFTFSSLNMF (SEQ ID NO:26), GFTFGSFNMF (SEQ ID NO:27), GFTFSSCNMF (SEQ ID NO:28) or GFTFSSFNMI (SEQ ID NO: 128), (v) the CDR-H2 comprises the sequence QIIDGAGSRTAYGAAVKG (SEQ ID NO:13), QISDGAGSRTAYGAAVKG (SEQ ID NO:22), QSIDGAGSRTAYGAAVKG (SEQ ID NO:29), QMIDGAGSRTAYGAAVKG (SEQ ID NO:30), QIIDGAGSRTADGAAVKG (SEQ ID NO:31), QIIEGAGSRTAYGAAVKG (SEQ ID NO:32) or ASICTGGSYTYYAPAVKG (SEQ ID NO:129), and (vi) the CDR-H3 comprises the sequence GGHYWGGASIDA (SEQ ID NO:14) or SVDGVGWHAGQIDA (SEQ ID NO:130).

9. The antibody or antigen binding fragment thereof according to claim 2, which is an IgY antibody or an antigen binding fragment thereof.

10-15. (canceled)

16. The antibody or antigen binding fragment thereof according to claim 9, wherein the antibody or antigen-binding fragment thereof comprises a variable light chain (V_{L}) comprising the sequence
(SEQ ID NO: 108)
ALTQPTSVSANLGGTVEITCSGGGSTDAGSYYYGWFQQKSPGSAPVTVI
YFNDKRPSDIPSRFSGSTSGSTNTLTITGVQADDEAVYFCGSADSTGGI
FGAGTTLTVL
or
(SEQ ID NO: 139)
ALTQPSSVSANLGGTVKITCSGSGSSWHGYGWYQQKAPGSAPVTVIYDN
TNRPSNIPSRFSGSASGSTATLTITGVRAEDEAVYFCGGYDSSTYAGIF
GAGTTLTVL.

17. (canceled)

18. The antibody or antigen binding fragment thereof according to claim 9, wherein the antibody or antigen-binding fragment thereof comprises a variable heavy chain (V_{H}) comprising the sequence
(SEQ ID NO: 109)
AVTLDESGGGLQTPGGALSLVCKASGFTFSSFNMFWVRQAPKGLEFVAQ
IIDGAGSRTAYGAAVKGRATISRDNGQSTVRLQLNNLRAEDTGTYYCAR
GGHYWGGASIDAWGHGTEVIVSS
or
(SEQ ID NO: 140)
AVTLDESGGGLQTPGRALSLVCKASGFTFSSFNMIWVRQTPGKGLEWVA
SICTGGSYTYYAPAVKGRATISRDNGQSTVRLQLNNLRAEDTATYFCAK
SVDGVGWHAGQIDAWGHGTEVIVSS.

19. (canceled)

20. The antibody or antigen binding fragment thereof according to claim 2, which is a humanized form of an IgY antibody or an antigen binding fragment thereof.

21-26. (canceled)

27. The antibody or antigen binding fragment thereof according to claim 20, wherein the antibody or antigen-binding fragment thereof comprises a variable light chain (V_{L}) comprising the sequence
(SEQ ID NO: 118)
SSELTQDPAVSVALGQTVRITCSGGGSTDAGSYYYGWYQQKPGQAPVTV
IYFNDKRPSGIPDRFSGSSSGNTASLTITGAQAEDEADYYCGSADSTGG
IFGGGTKLTVL
or
(SEQ ID NO: 141)
SSELTQPPAVSVALGQTVRITCSGSGSSWHGYGWYQQKPGQAPVTVIYD
NTNRPSGIPDRFSGSSSGNTASLTITGAQAEDEADYYCGGYDSSTYAGI
FGGGTKLTVL.

28. (canceled)

29. The antibody or antigen binding fragment thereof according to claim 20, wherein the antibody or antigen-binding fragment thereof comprises a variable heavy chain (V_{H}) comprising the sequence
(SEQ ID NO: 119)
EVQLLESGGGLVQPGGSLRLSCAASGFTFSSFNMFWVRQAPGKGLEWVA
QIIDGAGSRTAYGAAVKGRFTISRDNSKNTLYLQMNSLRAEDTAVYYCA
KGGHYWGGASIDAWGQGTLVTVSS
or
(SEQ ID NO: 142)
EVQLLESGGGLVQPGGSLRLSCAASGFTFSSFNMIWVRQAPGKGLEWVA
SICTGGSYTYYAPAVKGRFTISRDNSKNTLYLQMNSLRAEDTAVYYCAK
SVDGVGWHAGQIDAWGQGTLVTVSS.

30. (canceled)

31. (canceled)

32. The antibody or antigen binding fragment thereof according to claim 2, wherein said antigen binding fragment is a single-chain variable fragment (scFv) comprising a light chain variable region (V_{L}), a heavy chain variable region (V_{H}), and a linker connecting the V_{L} and the V_{H}.

33-39. (canceled)

40. The antibody or antigen binding fragment thereof according to claim 2, wherein the antibody or antigen-binding fragment thereof further comprises at least one constant domain or a fragment thereof.

41-48. (canceled)

49. A method of detecting NTSR1 in a sample or on a cell comprising contacting the cell with the antibody or antigen-binding fragment thereof according to claim 2, or the composition according to claim or 48.

50. A method for inhibiting NTSR1 activity in a cell, the method comprising contacting the cell with an effective amount of the antibody or antigen-binding fragment thereof according to claim 2, or the composition according to claim or 48.

51. (canceled)

52. A method for treating a disease or condition associated with NTSR1 activity in a subject, the method comprising administering to the subject an effective amount of the anti-NTSR1 antibody or antigen-binding fragment thereof according to claim 2.

53-65. (canceled)

66. A cyclic peptide comprising a domain of the following sequence:
X¹—X²—X³—N—R—S-A-D-G¹-X⁴—H—X⁵-G²-G³
wherein
“—” are bonds, preferably peptide (amide) bonds;
X¹ is an amino acid or amino acid analog or is absent;
X² is an amino acid or amino acid analog or is absent;
X³ is an amino acid or amino acid analog or is absent;
X⁴ and X⁵ are any amino acid,
one of X³ or N is attached via a bond, preferably a peptide bond, to G³, thereby forming a cyclic peptide;
or a salt thereof.

67. The cyclic peptide or salt thereof of claim 66, wherein said cyclic peptide has the following structure:

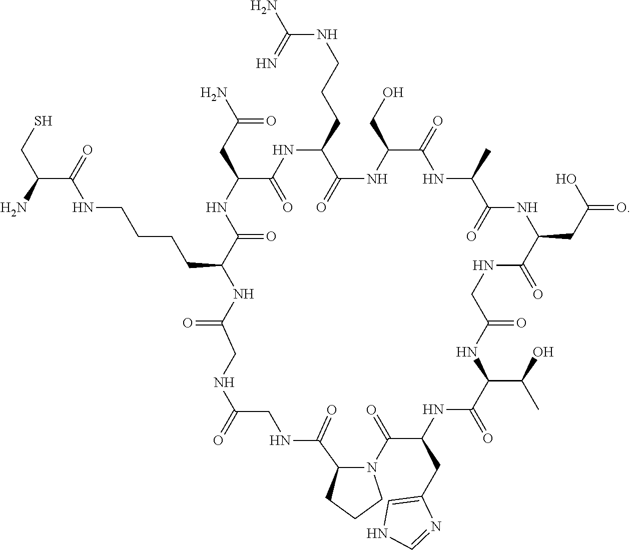 [SMILES: C[C@@H]1NC(=O)[C@H](CO)NC(=O)[C@H](CCCNC(=N)N)NC(=O)[C@H](CC(N)=O)NC(=O)[C@H](CCCCNC(=O)[C@@H](N)CS)NC(=O)CNC(=O)CNC(=O)[C@@H]2CCCN2C(=O)[C@H](Cc2c[nH]cn2)NC(=O)[C@H]([C@H](C)O)NC(=O)CNC(=O)[C@H](CC(=O)O)NC1=O]

68-77. (canceled)

78. The cyclic peptide or salt thereof of claim 66, which is conjugated to a carrier protein.

79. (canceled)

80. A composition comprising the cyclic peptide or salt thereof of claim 66 and an excipient, and optionally a vaccine adjuvant.

81-85. (canceled)

86. A method for inducing the production of an antibody that specifically binds to NTSR1 in an animal, the method comprising administering to said animal an effective amount of (i) the cyclic peptide or salt thereof according to claim 66, or (ii) a composition comprising said cyclic peptide or salt thereof and an excipient, and optionally a vaccine adjuvant, to said animal.

87-91. (canceled)
